(19) United States
(12) Patent Application Publication
(10) Pub. No.: US 2024/0010694 A1
(43) Pub. Date: Jan. 11, 2024

(54) HYDROGELS CONTAINING AFFIBODIES AND USES THEREOF

(71) Applicant: University of Oregon, Eugene, OR (US)

(72) Inventors: Marian Hettiaratchi, Eugene, OR (US); Jonathan Dorogin, Eugene, OR (US); Henry Hochstatter, Medford, MA (US); Veronica Spaulding, Harrisburg, OR (US); Alycia Galindo, Eugene, OR (US); Chandler Asnes, Eugene, OR (US); Justin Svendsen, Eugene, OR (US); Madeline Martin, Eugene, OR (US)

(73) Assignee: University of Oregon, Eugene, OR (US)

(21) Appl. No.: 18/340,754

(22) Filed: Jun. 23, 2023

Related U.S. Application Data
(60) Provisional application No. 63/359,723, filed on Jul. 8, 2022.

(51) Int. Cl. C07K 14/51; C07K 14/475; A61P 9/00; A61P 19/00; C07K 14/54; C07K 14/535

(52) U.S. Cl. CPC C07K 14/51; C07K 14/475; A61P 9/00; A61P 19/00; C07K 14/5406; C07K 14/535; C07K 2318/00

(57) ABSTRACT

Provided are unique affibodies specific for bone morphogenetic protein 2 (BMP-2), vascular endothelial growth factor (VEGF), fibroblast growth factor 2 (FGF-2), platelet-derived growth factor (PDGF), granulocyte-macrophage colony-stimulating factor (GM-CSF), inteleukin-4 (IL-4), and glial derived neurotrophic factor (GDNF), and well as hydrogels that include the affibodies and the corresponding protein. Also provided are methods of using the hydrogels, for example to treat bone injury, wounds, and neuron injury. In some examples, the hydrogel includes at least two different affibodies specific for the same protein, but have different disassociation constants (K_{D}).

### CROSS REFERENCE TO RELATED APPLICATIONS

This claims the benefit of U.S. Provisional Application No. 63/359,723, filed Jul. 8, 2022, which is hereby incorporated by reference in its entirety.

### ACKNOWLEDGMENT OF GOVERNMENT SUPPORT

This invention was made with government support under contract No. 1R21EB032112 awarded by the National Institutes of Health and under contract No. W81XWH-22-1-0700 awarded by the Department of Defense. The government has certain rights in the invention.

### FIELD

Provided are unique affibodies and hydrogels that include the affibodies and the corresponding protein, and methods of their use. In some examples, the affibodies in the hydrogel are specific for the same protein, but have different disassociation constants.

### INCORPORATION OF SEQUENCE LISTING

The Sequence Listing is submitted as an XML file in the form of the file named “sequence.xml” (˜70,285 bytes), which was created on Jun. 20, 2023 which is incorporated by reference herein.

### BACKGROUND

Bone morphogenetic protein-2 (BMP-2) is an integral protein for bone and cartilage repair.^{1,2} It has chemotactic properties that aid in the recruitment of osteoblasts and mesenchymal stromal cells,³⁻⁵ as well as morphogenic properties that differentiate mesenchymal stromal cells towards osteogenic phenotypes.^{1,6} Because of its ability to promote bone formation, BMP-2 has been used clinically as a bone graft substitute that is delivered from an implanted absorbable collagen sponge.^{2,7} However, the absorbable collagen sponge relies primarily on weak electrostatic interactions to physically entrap BMP-2, giving it a limited ability to retain BMP-2 compared to other materials.^{8,9} Uncontrolled release of BMP-2 from collagen sponges has led to reduced efficiency of BMP-2-mediated osteogenesis¹⁰ and numerous adverse effects, including soft tissue inflammation and ectopic bone formation.^{2,11,12} Consequently, there is a need to develop methods to improve control over BMP-2 delivery to improve the efficacy and reduce the side effects of clinical bone regeneration therapies.

Methods to control protein release from biomaterial delivery vehicles include physical modifications such as changing the porosity or degradation rate of the delivery vehicle^{13,14} and chemical modifications such as tethering proteins directly to the delivery vehicle.¹⁵ While promising under certain conditions, these techniques often result in burst release kinetics, unpredictable protein release rates within complex in vivo environments, and inconsistent loading of the protein therapeutic, contributing to insufficient localization of the protein in the intended site and poor healing outcomes.¹⁶ Although chemical conjugation of therapeutic proteins to biomaterials can reduce the likelihood of burst release and prolong protein presentation within the site of interest,¹⁷ it can also interfere with protein-receptor binding, potentially altering the biological function of the therapeutic protein and resulting in reduced protein bioactivity.¹⁸⁻²⁰

To better address the need for controlled protein delivery, biomaterial delivery vehicles have been fabricated from extracellular matrix molecules, such as heparin^{21,22} and fibronectin²³ with intrinsic affinity interactions for therapeutic proteins in an effort to provide sustained protein delivery without directly conjugating the protein to the delivery vehicle.^{11,24,25} Affinity-mediated protein release relies primarily on the equilibrium dissociation constant (K_{D}) between the protein and material to control the rate of protein release.²⁶ These reversible interactions provide prolonged protein presentation within the site of biomaterial implantation that mimics that of the extracellular matrix. Additional affinity interactions for therapeutic proteins have been engineered using various types of affinity molecules, including antibodies,²⁷ antibody fragments,⁷ peptides,^{28,29} and aptamers.³⁰

A challenge remains in finding ideal candidates to engineer suitable protein-material interactions for affinity-based release. Heparin-containing delivery vehicles have an intrinsic affinity for BMP-2 and the ability to retain BMP-2 at the site of injury^{7,31}; however, heparin interacts with a plethora of other proteins and extracellular matrix molecules via electrostatic and hydrophobic interactions,³² making it difficult to predict the behavior of the delivery vehicle in complex in vivo environments that contain numerous serum-borne proteins. While antibodies are highly specific protein binders with high affinities for their targets, they are expensive to produce and bulky, which hinders the integration of sufficient quantities of antibodies into a delivery vehicle.^{33 34} Conversely, aptamers, peptides, and antibody fragments are smaller and less expensive to produce, but may suffer from lower specificity and/or weaker affinities for their respective proteins due to either smaller interfaces of interaction^{35,36} or non-specific targeting domains such as the heparin-binding domain.^{32,37}

Directed evolution can be used to identify highly specific protein binders.³⁸ For instance, a large yeast surface display library containing ˜10⁸ unique protein binders was subjected to directed evolution to identify highly specific protein binding partners that were integrated into methylcellulose and hyaluronic acid hydrogels to tune the delivery rates of several growth factors.^{39,40} Similarly, directed evolution of a phage display library of random 7-mer peptides identified several binding peptides for integration into polyethylene glycol (PEG)-based hydrogels for controlled release of neurotrophin-3.⁴¹

### SUMMARY

Uncontrolled bone morphogenetic protein-2 (BMP-2) release can lead to off-target bone growth and other adverse events. To tackle this challenge, yeast surface display was used to identify unique BMP-2-specific affibodies that bound to BMP-2 with different affinities. Biolayer interferometry revealed an equilibrium dissociation constant (K_{D}) of 10.7 nM for the interaction between BMP-2 and a high-affinity affibody (SEQ ID NO: 1), 10.4 nM for the interaction between BMP-2 and a medium-affinity affibody (SEQ ID NO: 2), and 34.8 nM for the interaction between BMP-2 and a low-affinity affibody (SEQ ID NO: 3). The low-affinity affibody-BMP-2 interaction also exhibited an off-rate constant that was an order of magnitude higher than the off-rate constants of the medium- and high-affinity affibodies. Computational modeling of affibody-BMP-2 binding predicted that the high- and low-affinity affibodies bind to two distinct sites on BMP-2 that function as different cell-receptor binding sites. BMP-2 binding to affibodies reduced expression of the osteogenic marker alkaline phosphatase (ALP) in C2C12 myoblasts. Affibody-conjugated polyethylene glycol-maleimide hydrogels increased uptake of BMP-2 compared to affibody-free hydrogels, and high-affinity hydrogels exhibited lower BMP-2 release into serum compared to low-affinity hydrogels and affibody-free hydrogels over four weeks. Loading BMP-2 into affibody-conjugated hydrogels prolonged the ALP activity of C2C12 myoblasts compared to soluble BMP-2. Similar studies were conducted to identify and test affibodies for other proteins, including granulocyte macrophage colony-stimulating factor (GM-CSF), vascular endothelial growth factor (VEGF), fibroblast growth factor-2 (FGF-2), platelet-derived growth factor (PDGF), IL-4, and glial derived neurotrophic factor (GDNF). This work demonstrates that affibodies with different affinities can modulate protein delivery and activity, providing an approach for controlling protein delivery in clinical applications.

The present disclosure provides compositions that include a hydrogel, one or more proteins, and one or more affibodies specific for the one or more proteins, wherein the protein and antibodies are incorporated into the hydrogel (referred to herein as a hydrogel-affibody composition). The affibodies and proteins can be incorporated within the hydrogel. In some examples, the one or more proteins are bound to the one or more affibodies. For example, if the affibody is specific for BMP-2, the hydrogel can include BMP-2 bound to one or more different BMP-2-specific affibodies. Such compositions can further include a pharmaceutically acceptable carrier, such as water or saline or a buffer. In some examples, such compositions can be used to control the release of proteins in the hydrogel, which are bound to the affibodies.

In some examples, the hydrogel-affibody composition includes one or more, two or more, or 3 or more of bone morphogenetic protein 2 (BMP-2) protein, vascular endothelial growth factor (VEGF) protein (such as VEGF₁₆₅), fibroblast growth factor 2 (FGF-2) protein, platelet-derived growth factor (PDGF) protein (such as PDGF-BB), granulocyte-macrophage colony-stimulating factor (GM-CSF) protein, inteleukin-4 (IL-4) protein, and glial derived neurotrophic factor (GDNF) protein, and corresponding affibodies specific for one or more of BMP-2, VEGF, FGF-2, PDGF, GM-CSF, IL-4, and GDNF. Exemplary affibody sequences that can be used are provided in SEQ ID NOS: 1-74. In some examples, the hydrogel-affibody composition includes at least two affibodies specific for at least two of BMP-2, VEGF (such as VEGF₁₆₅), FGF2, PDGF (such as PDGF-BB), GM-CSF, IL-4, and GDNF (and at least two of the corresponding proteins). In some examples, the hydrogel-affibody composition includes the following proteins and one or more specific affibodies for the protein: a) VEGF, FGF2, and PDGF-BB; b) GM-CSF; c) GDNF; d) VEGF, FGF2, PDGF-BB, and BMP-2; e)GM-CSF and IL-4; f) GM-CSF, IL-4 and MCP-1; or g) GM-CSF, IL-4, and BMP-2.

The hydrogels can include additional proteins and affibodies, such as collagen I, collagen III, and/or monocyte chemoattractant protein-1 (MCP-1). In some examples the hydrogel-affibody composition further includes one or more additional chemoattractant proteins (e.g., MCP-1, SDF-1a) and affibodies, cytokine proteins (e.g., IL-10) and affibodies, immunomodulatory proteins (e.g., IL-10, MCP-1, G-CSF) and affibodies, and/or morphogen proteins (e.g., NGF, NT-3, BDNF) and affibodies.

The hydrogel-affibody composition can include at least 1, at least 2, at least 3, at least 4, at least 5, at least 10, at least 15, at least 20, at least 30, at least 40, or at least 50 (such as 1, 2, 3, 4, 5, 6, 7, 8, 9, 10, 15, 20, 25, 30, 35, 40, 45, 50, 75, 100 or more) different affibodies specific for a single protein. In such examples, each unique affibody can have a unique affinity or K_{D} for the target protein, such as at least one with a low K_{D}/strong affinity (e.g., K_{D} about 10⁻⁹-10⁻⁸ M), at least one with a medium K_{D}/medium affinity (e.g., K_{D} about 10⁻⁷-10⁻⁶ M) and at least one with a higher K_{D}/weak affinity (e.g., K_{D} about 10⁻⁵-10⁻³ M). In some examples, each unique affibody has a unique affinity or K_{D} for the target protein, such as at least one with a low K_{D}/strong affinity (e.g., K_{D} about 10⁻⁹-10⁻⁸ M) and at least one with a higher K_{D}/weak affinity (e.g., K_{D} about 10⁻⁵-10⁻³ M). In some examples, each unique affibody has a K_{D} for the target protein that is at least an order of magnitude (e.g., at least about 10 fold) different from another unique affibody. Thus, in some examples a low K_{D}/strong affinity affibody has a K_{D} that is at least about 10 times greater than a medium K_{D}/medium affinity affibody, and a medium K_{D}/medium affinity affibody has a K_{D} that is at least about 10 times greater than a higher K_{D}/weak affinity affibody.

In some examples, the hydrogel-affibody composition includes at least 1, at least 2, at least 3, at least 4, at least 5, at least 10, at least 15, at least 20, at least 30, at least 40, or at least 50 (such as 1, 2, 3, 4, 5, 6, 7, 8, 9, 10, 15, 25, 30, 35, 40, 45, 50, 75, 100 or more) different affibodies, wherein each unique affibody is specific for a single protein. In some examples, combinations are used (e.g., two or more affibodies specific for protein 1, and two or more affibodies specific for protein 2, etc.). In examples, where 2 or more unique affibodies are present that are specific for the same protein, each unique affibody can have a distinct K_{D}, such as one with a higher and another with a lower K_{D} (such as at least 2-fold, at least 3-fold, at least 5-fold, or at least 10-fold lower.

In some examples, the hydrogel includes hyaluronic acid (HA), polyethylene glycol (PEG), PEG-Maleimide, modified hyaluronic acid (e.g., Norbornene-HA, norbornene-oxidized-HA or oxidized-HA, hydrazide-HA, methacryalate-HA), thiolated poly(E-caprolactone) (PCL-SH), thiolated poly(lactide-co-glycolide) (PLGA-SH), thiolated silk-fibroin, modified gelatin (methacrylate (GelMA), oxidized gelatin, gelatin norbornene), collagen, or combinations thereof.

Also provided are methods of using the disclosed compositions to treat a disease, by administering an effective amount of the composition to a subject in need thereof. Such administration can be systemic or localized. For example, a hydrogel including BMP-2 and BMP-2 affibodies can be used to treat a bone or cartilage injury, a hydrogel including VEGF and VEGF affibodies can be used to increase angiogenesis (e.g., to treat a vascular disease or injury or wound, such as peripheral artery disease, diabetic ulcer, or critical limb ischemia), a hydrogel including FGF-2 and FGF-2 affibodies can be used to increase angiogenesis (e.g., to treat a vascular disease or injury or wound, such as peripheral artery disease, diabetic ulcer, or critical limb ischemia), a hydrogel including PDGF and PDGF affibodies can be used to increase angiogenesis (e.g., to treat a vascular disease or injury or wound, such as peripheral artery disease, diabetic ulcer, or critical limb ischemia), a hydrogel including GM-CSF and GM-CSF affibodies can be used to increase angiogenesis and manipulate the immune response to injury/disease (e.g., to treat a bone injury, vascular disease or injury or wound, such as peripheral artery disease, diabetic ulcer, or critical limb ischemia), and a hydrogel including GDNF affibodies can be used to treat a central nervous system injury or disease (e.g., to treat a neurological disease or injury, such as stroke, spinal cord injury, traumatic brain injury, paralysis, Parkinson's Disease, Alzheimer's Disease, ALS).

Also provided are isolated affibodies having at least 90%, at least 95%, at least 96%, at least 97%, at least 98%, at least 99%, or 100% sequence identity to SEQ ID NOS: 1-74. In some examples, the affibody consists of any one of SEQ ID NOS: 1-74. In some examples, the affibody consists of any one of SEQ ID NOS: 1-74, with an additional amino acid on the C-terminus, such as Cys, Lys, Tyr, Try, or Phe. In some examples, the affibody is 58, 59, 60, 61 or 65 amino acids in length. In some examples, the affibody has 1, 2, 3, 4, 5 or 6 conservative amino acid substitutions.

The foregoing and other features of this disclosure will become more apparent from the following detailed description of several aspects which proceeds with reference to the accompanying figures.

### BRIEF DESCRIPTION OF THE DRAWINGS

FIGS. 1A-1E: Identification of BMP-2-specific affibodies using cell sorting of a yeast surface display library. FIGS. 1A-1B: Magnetic activated cell sorting. FIG. 1A: Schematic of MACS. Yeast induced to express surface-displayed affibodies were incubated with magnetic beads coated with tris or bovine serum albumin (BSA) for negative bead sorts or BMP-2 for positive bead sorts. Yeast that did not bind to the negative sort beads were transferred to a tube containing the BMP-2 beads. Yeast that bound to the BMP-2 beads were collected and expanded for the next round of cell sorting. FIG. 1B: Diversities of the sorted yeast libraries obtained after each round of MACS. Left y-axis (bar graph) depicts approximate ratio of BMP-2-specific to non-specific binders (i.e., positive-to-negative binder ratio). Right y-axis (line graph) depicts the diversity of the yeast library after each round of MACS. FIGS. 1C-1E: FACS plots and corresponding cell labeling diagrams. Yeast was incubated with secondary fluorescent tags that bound specifically to αCMYC for affibody expression or bBMP. FIG. 1C: Secondary fluorescent tag control, in which no affibody expression or bBMP-2 binding is measured. FIG. 1D: In the presence of αCMYC, affibody expression is observed as a rightward shift. FIG. 1E: In the presence of αCMYC and bBMP-2, affibody expression is observed as a rightward shift, and binding of bBMP-2 to displayed affibodies results in a shift upward along the y-axis. Gating was performed based on positive bBMP-2 binding and affibody expression (upper right quadrant).

FIGS. 2A-2B: Gating approaches for fluorescent-activated cell sorting of enriched affibody-displaying yeast library. FIG. 2A: Cell sorting based on ratio of BMP-2 binding to affibody expression. Gates were created for ratios of >1, ˜1, <1, based on our hypothesis that a higher ratio of BMP-2 binding to affibody expression would correspond to higher affinity for BMP-2. 8 unique affibody sequences were identified from 9 colonies picked from yeast growth plates after sorting. FIG. 2B: Cell sorting based on BMP-2 binding only. Gates were created based on the hypothesis that higher BMP-2 binding to the yeast would correspond to higher BMP-2 affinity. 3 unique affibody sequences were identified from the 12 colonies picked from yeast growth plates after sorting.

FIGS. 3A-3H: Identification and characterization of yeast-displayed BMP-2-specific affibodies. Affibody binding to BMP-2 and off-target proteins was determined using flow cytometry. Flow cytometry plots of affibodies binding to bBMP-2 at 0.5 nM of bBMP-2 (FIG. 3A), 5 nM of bBMP-2 (FIG. 3B), 50 nM of bBMP-2 (FIG. 3C), and 500 nM of bBMP-2 (FIG. 3D). FIG. 3E: Fraction of yeast binding to bBMP-2 as a function of bBMP-2 concentration ranging from 0.5-1000 nM. Non-linear regression was performed to determine equilibrium dissociation constants (K_{D}). Curves were identified as statistically significantly different from each other by two-way ANOVA, Tukey's post-hoc test. n=3, p<0.01. FIG. 3F: Equilibrium dissociation constants (K_{D}) of the three unique affibodies. Statistical significance was determined using one-way ANOVA with Tukey's post-hoc test. n=3, **p<0.01 FIGS. 3G-3H: BMP-2-specific affibodies do not bind to other recombinant proteins of interest. High-affinity (FIG. 3G) and low-affinity affibodies (FIG. 3H) were incubated with 1000 nM of bBMP-2, bVEGF, bIL-4, or bGM-CSF. Statistical significance was measured using one-way ANOVA and Tukey's post-hoc test. n=3, ****p<0.0001

FIGS. 4A-4B: bBMP-2 binding to selected affibody-displaying yeast clones from enriched yeast library. FIG. 4A: All 11 unique BMP-2 affibodies (SEQ ID NOS: 1-11) were assessed for their binding to bBMP-2 using yeast surface display, bBMP-2 concentrations ranging from 0.5-1000 nM, and flow cytometry. Yeast clones with names starting with the letter A were identified using the gating approach in FIG. 2A, and yeast clones with names starting with B were identified using the gate approach in FIG. 2B. The affibodies chosen for further characterization are boxed in the legend. FIG. 4B: Average AlexaFluor647 signal intensity for yeast displaying high-affinity and low-affinity BMP-2 affibodies at BMP-2 concentrations between 0.5 nM and 500 nM, as determined by flow cytometry. No significant differences were observed using a non-parametric multiple t test with Mann-Whitney post-hoc analysis. n=4, p=0.6857.

FIGS. 5A-5B: Soluble affibody characterization. FIG. 5A: SDS-PAGE of high- and low-affinity BMP-2-specific affibodies with a 5-245 kDa ladder. Samples were run at concentration of 150 μM. Expected molecular weights of the high- and low-affinity affibodies were 7308 Da and 7414 Da, respectively. FIG. 5B: Circular dichroism spectra of affibodies displayed in molar ellipticity. Affibodies were diluted to a concentration between 17-30 μM in 5 mM tris pH 6.92 and loaded into a quartz cuvette with 1 mm path length. Circular dichroism and high-tension voltage were measured over a wavelength range of 190-250 nm, and the circular dichroism output was adjusted for protein concentration and molecular weight.

FIG. 6: Deconvolved mass spectra relative abundance of high-affinity and low-affinity affibodies. Accurate masses were measured using deconvolved native mass spectrometry collected with Waters Synapt G2Si, using a CsI calibration profile. Purified affibodies were dissolved in 0.6 M tris pH 8 and buffer-exchanged into 0.2 M ammonium acetate pH 7.52. Mass spectra were collected over 1-5 minutes using nano-electrospray ionization at a capillary voltage of 0.7-1.0 kV. Samples were deconvolved in UniDec using charge states of 3 to 7 and masses of 5000-9000 Da. The high-affinity affibody's (SEQ ID NO: 1) most abundant peak was 7308 Da (starred), aligned with the expected mass within 2 Da. Mass spectrometry associated adducts of sodium (22 Da) and potassium (38 Da) are also visible. Other apparent peaks could be associated with dehydroalanine (−34 Da) and either a piperidine (51 Da) or cysteic acid formation (48 Da) on the C-terminal cysteine or glutamylation (129 Da (+/−2)) of the terminal cysteine. The low-affinity affibody (SEQ ID NO: 3) had a small peak at the expected mass of 7414 Da (starred), but had prominent peak shifts associated with the dehydroalanine, a 32 Da shift which could be indicative of a proline oxidation or 3,4-dihydroxylation, an additional shift associated with a piperidine formation, and another prominent peak (161 Da) which could be caused by a carboxymethyl cysteine or carboxymethyl cystenyl.

FIGS. 7A-7C: Binding interactions of soluble affibodies with BMP-2 measured by biolayer interferometry. All samples were diluted in PBST. Streptavidin probes were loaded with 25 nM of bBMP-2 for 120 seconds. Bound protein was allowed to associate with 0-125 nM high-affinity affibody (SEQ ID NO: 1) (FIG. 7A) and low-affinity affibody (SEQ ID NO: 3) (FIG. 7B) for 120 seconds followed by dissociation into PBST for 120 seconds. Association and dissociation rate constants as well as overall equilibrium dissociation constants were obtained using a 1:1 global curve fitting of the data. Raw data is displayed with solid lines, and fitted data is displayed as dotted lines. FIG. 7C: Table of association and dissociation rate constants and overall equilibrium dissociation constants of high-, medium- and low-affinity BMP-2 affibodies (SEQ ID NOS: 1, 2, and 3, respectively).

FIGS. 8A-8F: Binding interactions of soluble BMP-2-specific affibodies with VEGF, IL-4, and GM-CSF measured by biolayer interferometry. All samples were diluted in PBST. Streptavidin probes were loaded with 25 nM of bVEGF (FIGS. 8A & 8D), bIL-4 (FIGS. 8B & 8E), or bGM-CSF (FIGS. 8C & 8F) for 120 seconds. Bound protein was allowed to associate with 0-125 nM high-affinity (SEQ ID NO: 1) (FIGS. 8A-8C) or low-affinity (SEQ ID NO: 3) (FIGS. 8D-8F) affibodies for 120 seconds followed by dissociation into PBST for 120 seconds.

FIGS. 8G-8H: Binding interactions of soluble medium-affinity BMP-2 affibody (SEQ ID NO: 2) with BMP-2 and VEGF measured by biolayer interferometry. All samples were diluted in PBS solution containing 0.05% Tween-20® (PBST). Streptavidin probes were loaded with 25 nM biotinylated BMP-2 for on-target binding (FIG. 8G) and 25 nM biotinylated VEGF for off-target binding (FIG. 8H) for 120 seconds, excess protein was allowed to dissociate in PBST for 120 seconds, then bound protein was allowed to associate with 0-125 nM medium-affinity BMP-2 affibodies for 120 seconds and dissociate into PBST for 120 seconds. Dissociation constants were obtained from a 1:1 global curve fitting of the data. n=4.

FIGS. 9A-9B: Computational predictions of BMP-2 binding with affibodies or BMP receptors. FIG. 9A: Visual representation of docking of affibodies and receptors to BMP-2 in Pymol. High-affinity affibody (SEQ ID NO: 1) (a) overlaps with the docking interface of BMPR-1A (b), and low-affinity affibody (SEQ ID NO: 3) (c) overlaps with the docking interface of BMPRII (d). Predicted affibody structures were docked to BMP-2 using ZDOCK. FIG. 9B: Characteristic binding interactions of affibodies and BMP receptors to BMP-2. The high-affinity affibody (SEQ ID NO: 1) docks to BMP-2 in the binding epitope known at the “wrist” using 3 polar contacts and 5 hydrophobic interactions. The low-affinity affibody (SEQ ID NO: 3) docks to BMP-2 at the binding epitope called the “knuckle” with 3 polar interactions and one weak hydrophobic pocket. The wrist traditionally binds BMPR-1A (PDB: 1REW) and the knuckle traditionally binds with BMPR-II (PDB: 7PPA).

FIGS. 10A-10F: Effects of BMP-2 affibodies on viability, growth, and alkaline phosphate activity of C2C12 skeletal myoblasts. FIGS. 10A-10E: Cytocompatibility of high- and low-affinity affibodies with C2C12s. Cells were seeded at a density of 2000 cells cm⁻² and incubated with 0-800 nM soluble affibody in growth media for 72 hours. Cells were stained with calcein AM (live cells; light gray) and ethidium homodimer (dead cells; starred) and imaged. FIGS. 10A-10C: Representative photos of stained cell culture wells containing C2C12 cells and no affibodies (FIG. 10A), 20 nM high-affinity affibodies (FIG. 10B), and 20 nM low-affinity affibodies (FIG. 10C). Scale bar=1000 μm. FIG. 10D: Percent cell viability as a function of affibody concentration. Significance determined by two-way ANOVA and Dunnett's post-hoc test, n=4, *p<0.05. FIG. 10E: Total viable cell number as a function of affibody concentration. Statistical significance determined by two-way ANOVA and Dunnett's post-hoc test, n=4, *p<0.05. FIG. 10F: Effects of BMP-2 and BMP-2-specific affibodies on alkaline phosphatase activity of C2C12 cells. C2C12 cells were seeded at a density of 62,500 cells cm⁻² and allowed to adhere for 6 hours in growth media. Media was then replaced with low serum media containing premixed affibody (20 nM) and BMP-2 (20 nM) (complexed) or low serum media containing 20 nM soluble affibody with 20 nM of BMP-2 added 45 minutes later (uncomplexed). The cells were cultured for 72 hours and then lysed for quantification of ALP activity and double stranded DNA content. Statistical significance was determined by one-way ANOVA and Tukey's post-hoc test. n=4, *p<0.05, **p<0.01, ***p<0.001, ****p<0.0001. Complexed and uncomplexed affibody-BMP-2 structures were obtained from computational predictions.

FIG. 11: Effects of BMP-2 and BMP-2-specific affibodies on normalized alkaline phosphatase activity of C2C12 cells. C2C12 cells were seeded at an initial density of 62,500 cells/cm² and allowed to adhere for 6 hours in high serum media. Media were then replaced with low serum media containing premixed affibody (10-1000 nM) and BMP-2 (20 nM) (complexed) or low serum media containing 10⁻¹⁰⁰⁰ nM soluble affibody with 20 nM of BMP-2 added 45 min later (uncomplexed). The cells were cultured for 72 hours and then lysed. ALP activity and double stranded DNA content of lysates were quantified, and ALP activity was normalized to dsDNA content. Statistical significance was determined by one-way ANOVA and Tukey's post-hoc test. n=4, *p<0.05, **p<0.01, ***p<0.001, ****p<0.0001

FIGS. 12A-12E: Release of BMP-2 from affibody-conjugated PEG-Mal hydrogels. FIG. 12A: Synthesis schematic for affibody-conjugated PEG-Mal hydrogels. 4-arm PEG-Mal (20 kDa) underwent a thiol-maleimide Michael addition with the terminal cysteines of the high- or low-affinity affibodies in PBS pH 6.92. The remaining unreacted maleimide groups of the intermediate complex were crosslinked with DTT in PBS pH 6.92 to form a hydrogel. PEG-Mal hydrogels containing no affibody, low-affinity affibody, or high-affinity affibody were loaded with 100 ng of BMP-2. FIG. 12B: Encapsulation efficiency of affibody-conjugated PEG-Mal hydrogels. One-way ANOVA and Tukey's post-hoc test. n=8, ****p<0.0001. FIGS. 12C-12D: Cumulative BMP-2 release from affibody-conjugated PEG-Mal hydrogels measured over a 4-week period using ELISA. BMP-2 was released into either saline solution (FIG. 12C) or 10% serum solution (FIG. 12D). Two-way ANOVA and Tukey's post-hoc test. n=4. *p<0.05, **p<0.01. FIG. 12E Fickian diffusion rates of BMP-2 release from PEG-Mal hydrogels observed in the linear region of release in serum and saline solutions. One-way ANOVA and Tukey's post-hoc test. n=4 ****p<0.0001.

FIGS. 13A-13B: Confirmation of affibody-to-PEG-maleimide conjugation through SDS-PAGE. The unconjugated affibody solution and intermediate product (affibody-conjugated PEG-Mal) underwent centrifuge filtration (10 kDa MWCO filter, 5 min, 15000×g), and the flowthrough was subjected to SDS-PAGE. For each SDS-PAGE gel, the left lane is the ladder, middle lane contains the flowthrough of the unconjugated affibody solution after undergoing centrifuge filtration, and right lane contains the flowthrough of the intermediate product after undergoing centrifuge filtration, in which unconjugated affibody is not expected to be present. FIG. 13A: Conjugation of high-affinity affibody (SEQ ID NO: 1) to PEG-Mal. FIG. 13B: Conjugation of low-affinity affibody (SEQ ID NO: 3) to PEG-Mal.

FIGS. 14A-D: ALP activity of C2C12 cells as a function of BMP-2 release from affibody-conjugated PEG-Mal hydrogels. PEG-Mal hydrogels containing no affibody, low-affinity affibody (SEQ ID NO: 3), or high-affinity affibody (SEQ ID NO: 1) were loaded with 200 ng of BMP-2. The hydrogels were submerged in low serum media, and aliquots of the media were removed and replenished with fresh media over 7 days. FIG. 14A: Amount of BMP-2 added to C2C12 cells from each timepoint was quantified by BMP-2 ELISA. Two-way ANOVA and Tukey's post-hoc test. n=4, *p<0.05, **p<0.01, ***p<0.001. FIG. 14B: ALP activity of C2C12 myoblasts normalized to double-stranded DNA content of the cell cultures. 180 μL of media from each timepoint of BMP-2 release were added to C2C12 cells seeded at 62,500 cells cm⁻² and allowed to incubate for 72 h at 37° C., after which ALP activity was quantified and normalized to dsDNA. FIG. 14C: ALP activity of BMP-2 normalized to the amount of BMP-2 in solution at each timepoint. Two-way ANOVA and Tukey's post-hoc test. n=4, *p<0.05. FIG. 14D: Area Under the Curve (AUC) of the normalized ALP activity from FIG. 14C for each PEG-Mal hydrogel and the soluble BMP-2 control. One-way ANOVA, post-hoc Tukey multiple comparisons. n=4, *p<0.05, **p<0.01, ***p<0.001, ****p<0.0001.

FIG. 15: BMP-2 release from affibody-conjugated PEG-Mal hydrogels into high glucose DMEM supplemented with 1% fetal bovine serum for C2C12 alkaline phosphatase assays. PEG-Mal hydrogels containing no affibody, low-affinity affibody, or high-affinity affibody were loaded with 200 ng of BMP-2. The hydrogels were submerged in low serum media, and aliquots of the media were removed and replenished with fresh media over 7 days. Release was measured using BMP-2-specific ELISA.

FIGS. 16A-16C: Release of BMP-2 from hydrogels. FIG. 16A: Schematic of synthesis of affibody conjugated hydrogel. FIG. 16B: High-affinity hydrogels exhibited lower BMP-2 release into serum compared to low-affinity hydrogels and affibody-free hydrogels over four weeks. FIG. 16C: Schematic of affibody-BMP-2 binding modulating osteogenic bioactivity.

FIGS. 17A-17D show conjugation of BMP-2 specific affibody (SEQ ID NO: 1) to hyaluronic acid (HA) hydrogel. FIG. 17A: Synthetic scheme of BMP-2 affibody conjugation to HA hydrogel. FIG. 17B: Cumulative BMP-2 release from hydrogel with (bottom) and without (top) affibody. FIG. 17C: Linearized release of BMP-2 from hydrogel with (bottom) and without (top) affibody. FIG. 17D: Slope of fractional releases show significance. ****indicates p<0.0001.

FIGS. 18A-18D show characterization of GM-CSF affibodies. FIG. 18A: yeast displaying GM-CSF affibodies incubated with increasing amounts of biotinylated GM-CSF. FIG. 18B: Yeast displaying GM-CSF affibodies incubated with increasing amounts of biotinylated high-affinity GM-CSF affibody (SEQ ID NO: 12). FIG. 18D: Yeast displaying GM-CSF affibodies incubated with increasing amounts of biotinylated medium-affinity GM-CSF affibody (SEQ ID NO: 13). FIG. 18D: Yeast displaying GM-CSF affibodies incubated with increasing amounts of biotinylated low-affinity GM-CSF affibody (SEQ ID NO: 14).

FIGS. 19A-19C are graphs showing yeast displaying high-, medium-, and low-affinity affibodies incubated with increasing amounts of biotinylated FIG. 19A: VEGF, FIG. 19B: FGF-2, and FIG. 19C PDGF.

FIGS. 19D-19F are graphs showing yeast displaying high-, medium-, and low-affinity affibodies incubated with increasing amounts of biotinylated FIG. 19D: VEGF, FIG. 19E: FGF-2, and FIG. 19F PDGF, showing specificity of each affibody for its target protein and any off-target binding to other proteins.

FIG. 19G is a graph showing biolayer interferometry data characterizing the dissociation constant between PDGF and a high-affinity PDGF affibody (SEQ ID NO: 59).

FIGS. 20A-20B show conjugation of a GDNF-specific affibody to hyaluronic acid (HA) and alginate hydrogel.

FIG. 21 is a graph showing release of BMP-2 over time in vivo, in the presence of no affibody, or the affibody of SEQ ID NO: 1, 2, or 3. Fluorescent BMP-2 was in subcutaneous space in vivo in the presence of affibody. n=6.

FIGS. 22A-22B: Controlled co-delivery of BMP-2 and IL-4 from dual-affibody-conjugated PEG-maleimide hydrogels. FIG. 22A: PEG-Mal hydrogels conjugated with no affibody, high- or low-affinity BMP-2 affibody and/or high- or low-affinity IL-4 affibody were synthesized as described. Briefly, 4-arm PEG-Mal was mixed with no affibody, high- or low-affinity BMP-2 affibody and/or high- or low-affinity IL-4 affibody to form affibody-conjugated intermediate solutions, and then crosslinked with DTT to form affibody-conjugated hydrogels. FIG. 22B: Hydrogels were loaded with 50 ng each of BMP-2 and IL-4 and aliquots were taken over 7 days. BMP-2 and IL-4 release was quantified by protein-specific ELISA. n=4

FIG. 23 is a graph showing FGF-2 release from PEG-Mal hydrogels containing FGF-2 and no affibodies, or in the presence of low—(SEQ ID NO: 44), medium—(SEQ ID NO: 43), or high-(SEQ ID NO: 42) affinity FGF-2 affibodies.

FIG. 24 is a graph showing PDGF release from PEG-Mal hydrogels containing PDGF and no affibodies, or in the presence of medium—(SEQ ID NO: 60), or high-(SEQ ID NO: 59) affinity PDGF affibodies.

FIGS. 25A-25B: Schematic drawings providing overview of technology. FIG. 25A: Exemplary hydrogel that includes an affibody (3 helices) specific for a target (triangle), and schematic showing release of protein from hydrogel depending on the affinity of the affibody for the protein. FIG. 25B: Exemplary hydrogel that includes three different affibodies (3 helices) specific for three different proteins (triangle, rod, half circle), wherein each affibody has a different affinity for its corresponding protein, and schematic showing release of protein from hydrogel depending on the affinity of the affibody for the protein (triangle weak affinity, rod medium affinity, half circle strong affinity).

### SEQUENCE LISTING

The amino acid sequences provided herein are shown using standard three letter code for amino acids, as defined in 37 C.F.R. 1.822.

SEQ ID NOs: 1-11 are exemplary BMP-2 affibody sequences.

SEQ ID NOs: 12-19 are exemplary GM-CSF affibody sequences.

SEQ ID NOs: 20-41 are exemplary VEGF-165 affibody sequences.

SEQ ID NOs: 42-56 are exemplary FGF affibody sequences.

SEQ ID NOs: 57-60 are exemplary PDGF-BB affibody sequences.

SEQ ID NOS: 61 to 64 are exemplary IL-4 affibody sequences.

SEQ ID NOS: 65 to 70 are exemplary glial derived neurotrophic affibody sequences.

SEQ ID NOS: 71-73 are exemplary BMP-2 affibody sequences with a hexahistidine tag and C-terminal cysteine.

SEQ ID NO: 74 is an exemplary GM-CSF affibody sequence with a hexahistidine tag and C-terminal cysteine.

SEQ ID NOS: 75 and 76 are primer sequences.

### DETAILED DESCRIPTION

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. “Comprising” means “including.” Hence “comprising A or B” means “including A” or “including B” or “including A and B.”

Suitable methods and materials for the practice and/or testing of embodiments of the disclosure are described below. Such methods and materials are illustrative only and are not intended to be limiting. Other methods and materials similar or equivalent to those described herein can be used.

The sequences associated with all GenBank® Accession numbers referenced herein are incorporated by reference for the sequence available on Jul. 8, 2022.

In order to facilitate review of the various embodiments of the disclosure, the following explanations of specific terms are provided:

Administration: Administration of a composition, such as a hydrogel-affibody composition provided herein, can be by any route known to one of skill in the art. Administration can be local or systemic. Examples of local administration include, but are not limited to, topical administration, subcutaneous administration, intramuscular administration, intrathecal administration, intrapericardial administration, intra-ocular administration, topical ophthalmic administration, administration to a bone (e.g., intraosseous), administration to a tumor, administration to a wound, or administration to the nasal mucosa or lungs by inhalational administration. In addition, local administration includes routes of administration typically used for systemic administration, for example by directing intravascular administration to the arterial supply for a particular organ. Thus, in particular embodiments, local administration includes intra-arterial administration and intravenous administration when such administration is targeted to the vasculature supplying a particular organ. Local administration also includes the incorporation of active compounds and agents into implantable devices or constructs, such as vascular stents or other reservoirs, which release the active agents and compounds over extended time intervals for sustained treatment effects. In one example, administration is oral.

Systemic administration includes any route of administration designed to distribute an active compound or composition widely throughout the body via the circulatory system. Thus, systemic administration includes, but is not limited to intra-arterial and intravenous administration. Systemic administration also includes, but is not limited to, topical administration, subcutaneous administration, intramuscular administration, or administration by inhalation, when such administration is directed at absorption and distribution throughout the body by the circulatory system.

Affibody: A small protein that binds to a target proteins or peptides with varying affinity, and are therefore a member of the family of antibody mimetics. In some examples, affibody molecules include alpha helices and lack disulfide bridges. For example, an affibody can include three alpha helices with 58 amino acids, having a molar mass of about 6 kDa. In some examples, different affibodies specific for one protein each have a different K_{D} such as strong/high (10⁻⁹-10⁻⁸ M), medium (10⁻⁷-10⁻⁶ M) and weak (10⁻⁵-10⁻³ M) affinity.

Binding affinity: Affinity of an antibody or other antigen-binding molecule (such as an affibody for a protein). Affinity can be quantified by calculating a dissociation constant, K_{D}.

An affibody that “specifically binds” a protein (such as BMP-2, VEGF, FGF-2, PDGF, GM-CSF, IL-4, or GDNF) is an affibody that binds the protein with high affinity and does not significantly bind other unrelated proteins. In some examples, an affibody specifically binds to a target protein with weak affinity, such as with a K_{D} that is no more than 10⁻⁵ M, such as no more than 10⁻⁴ M, no more than 10⁻³ M, or no more than 10⁻² M, such as about 10⁻⁵-10⁻³ M. In some examples, an affibody specifically binds to a target with moderate or medium affinity, such as with a K_{D} that is no more than 10⁻⁷ M, such as no more than 10⁻⁶ M, such as about 10⁻⁷-10⁻⁶ M. In some examples, an affibody specifically binds to a target with high or strong affinity, such as with a K_{D} that is at least 10⁻¹⁰ M, such as at least 10⁻⁹ M, or at least 10⁻⁸ M, such as about 10⁻¹⁰-10⁻⁸ M.

Bone: A rigid organ that constitutes part of the skeleton in most vertebrate animals. Bones protect the various other organs of the body, produce red and white blood cells, store minerals, provide structure and support for the body, and enable mobility. The disclosed compositions can be used to treat a bone injury, such as a fracture, for example in the spinal column, vertebrae (such as the lumbar vertebra), femur, tibia, fibula, thoracic cage, rib, clavicle, humerus, radius, ulna, tarsal bone, ilium, cranium or carpal bone.

Bone morphogenetic protein 2 (BMP-2): (e.g., OMIM 112261) A bone morphogenetic protein that plays a role in the development of bone and cartilage. It is involved in the hedgehog pathway, TGF beta signaling pathway, and in cytokine-cytokine receptor interaction. It is also involved in cardiac cell differentiation and epithelial to mesenchymal transition. Thus, PDGF affibodies can be used to control the release of BMP-2 and treat a bone injury. Exemplary BMP-2 sequences can be found in the GenBank® database (e.g., Accession Nos. NP_001191.1, AGG86667.1, NM_001200.4, NP_031579.2, and CAA81088.1). In some examples, a BMP-2 protein or coding sequence has at least 80%, at least 90%, at least 95%, at least 96%, at least 97%, at least 98%, at least 99% or 100% sequence identity to the sequence provided in NP_001191.1, AGG86667.1, NM_001200.4, NP_031579.2, or CAA81088.1.

Bone repair or regeneration: Includes osteogenesis, bone regeneration, bone repair, bone reformation, and bone remodeling.

Contacting: Placement in direct physical association; includes both in solid and liquid form.

Conservative variant: A protein, such as an affibody, containing conservative amino acid substitutions that do not substantially affect or decrease the affinity of an affibody for its corresponding protein. Conservative amino acid substitutions are those substitutions that, when made, least interfere with the properties of the original protein, that is, the structure and especially the function of the protein is conserved and not significantly changed by such substitutions. For example, an affibody provided herein that specifically binds to its corresponding protein can include at most about 1, at most about 2, at most about 5, and most about 10, or at most about 15 conservative substitutions and specifically bind the protein with a similar K_{D} (e.g., a change of no more than 10%, no more than 5%, or no more than 1%) than the original sequence. The term “conservative variant” also includes the use of a substituted amino acid in place of an unsubstituted parent amino acid, provided that the affibody specifically binds to its corresponding protein.

Conservative amino acid substitution tables providing functionally similar amino acids are well known. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Consists Of: A polypeptide of a specified amino acid sequence (such as an affibody sequence) that does not include any additional amino acid residues. The residues in the polypeptide can be modified to include non-peptide components. The N- and/or C-terminus of a polypeptide that consists of a specified amino acid sequence can be joined (for example, by a covalent bond) to a chemical linker for conjugation chemistry. A polypeptide that consists of a specified amino acid sequence can be glycosylated and/or can include non-naturally occurring amino acids.

Dissociation constant (K_{D}): The concentration of ligand/affibody, wherein half of the ligand/affibody binding sites on the protein are occupied in the system equilibrium. It is calculated by dividing the k_{off} value by the kₒₙ value. The smaller the K_{D} value, the greater the binding affinity of the ligand/affibody for its target protein. The larger the K_{D} value, the more weakly the target protein and ligand/affibody are attracted to and bind to one another.

Numerous methods are available to calculate the K_{D} value for an affibody, and the disclosure is not limited to a particular method. In one embodiment, K_{D} is calculated by a modification of the Scatchard method described by Frankel et al., Mol. Immunol., 16:101-106, 1979. Other exemplary methods include competition radioimmunoassay, ELISA, flow cytometry, and surface plasmon resonance assays (e.g., using a BIACORES-2000 or a BIACORES-3000 (BIAcore, Inc., Piscataway, N.J.)). In some embodiments, K_{D} is measured using the Octet system (ForteBio), which is based on bio-layer interferometry (BLI) technology.

In some examples, an affibody has a K_{D} of 1 nM or less. In some examples, an affibody binds to a target protein, such as BMP-2, with a K_{D} of at least about 10⁻³ M, at least about 10⁻⁴ M, at least about 10⁻⁵ M, at least about 10⁻⁶ M, at least about 10⁻⁷ M, at least about 10⁻⁸ M, at least about 10⁻⁹ M, or at least about 10⁻¹⁰ M.

Effective amount: An amount of agent, such as a hydrogel-affibody composition provided herein, that is sufficient to elicit a desired response, such as treating a bone injury, wound, vascular disease, or neurological disease/disorder in a subject. It is understood that to obtain an effect, a method can require multiple administrations of a disclosed hydrogel-affibody composition. In one example, a desired response is to manipulate the immune response, increase wound healing, increase bone injury healing, increase angiogenesis, increase recruitment and differentiation of immune cells, increase recruitment and differentiation of osteogenic cells, increase neuron survival and/or increase neurological growth. The wound, disease, disorder, or injury does not need to be completely eliminated or reduced or prevented for the method to be effective. In one example, administration of a therapeutically effective amount of the hydrogel-affibody composition increases the rate of wound healing and/or the amount of wound healing, for example by at least 10%, at least 20%, at least 50%, at least 60%, at least 70%, at least 80%, at least 85%, at least 90%, at least 95%, at least 98%, at least 99% or even at least 100% (complete healing of the wound), for example as compared to a suitable control, such as the absence of the hydrogel-affibody composition. In one example, administration of a therapeutically effective amount of the hydrogel-affibody composition increases the rate of healing of a bone injury and/or the amount of bone injury, for example by at least 10%, at least 20%, at least 50%, at least 60%, at least 70%, at least 80%, at least 85%, at least 90%, at least 95%, at least 98%, at least 99% or even at least 100% (complete healing of the bone injury), for example as compared to a suitable control, such as the absence of the hydrogel-affibody composition. In one example, administration of a therapeutically effective amount of the hydrogel-affibody composition increases the rate and/or amount of differentiation of osteogenic cells, for example by at least 10%, at least 20%, at least 50%, at least 60%, at least 70%, at least 80%, at least 85%, at least 90%, at least 95%, at least 98%, at least 99%, at least 100%, at least 200%, at least 300%, or at least 500% (for example as compared to a suitable control, such as the absence of the hydrogel-affibody composition). In one example, administration of a therapeutically effective amount of the hydrogel-affibody composition increases angiogenesis, for example by at least 10%, at least 20%, at least 50%, at least 60%, at least 70%, at least 80%, at least 85%, at least 90%, at least 95%, at least 98%, at least 99%, at least 100%, at least 200%, at least 300%, or at least 500% (for example as compared to a suitable control, such as the absence of the hydrogel-affibody composition). In one example, administration of a therapeutically effective amount of the hydrogel-affibody composition increases recruitment and/or differentiation of immune cells, for example by at least 10%, at least 20%, at least 50%, at least 60%, at least 70%, at least 80%, at least 85%, at least 90%, at least 95%, at least 98%, at least 99%, at least 100%, at least 200%, at least 300%, or at least 500% (for example as compared to a suitable control, such as the absence of the hydrogel-affibody composition). In one example, administration of a therapeutically effective amount of the hydrogel-affibody composition increases neuron survival, for example by at least 10%, at least 20%, at least 50%, at least 60%, at least 70%, at least 80%, at least 85%, at least 90%, at least 95%, at least 98%, at least 99% at least 100%, at least 200%, at least 300%, or at least 500% (for example as compared to a suitable control, such as the absence of the hydrogel-affibody composition). In one example, administration of a therapeutically effective amount of the hydrogel-affibody composition increases neuron growth, for example by at least 10%, at least 20%, at least 50%, at least 60%, at least 70%, at least 80%, at least 85%, at least 90%, at least 95%, at least 98%, at least 99% at least 100%, at least 200%, at least 300%, or at least 500% (for example as compared to a suitable control, such as the absence of the hydrogel-affibody composition). In one example, administration of a therapeutically effective amount of the hydrogel-affibody composition increases the proliferation of new neurons, for example by at least 10%, at least 20%, at least 50%, at least 60%, at least 70%, at least 80%, at least 85%, at least 90%, at least 95%, at least 98%, at least 99% at least 100%, at least 200%, at least 300%, or at least 500% (for example as compared to a suitable control, such as the absence of the hydrogel-affibody composition).

A therapeutically effective amount of a hydrogel-affibody composition provided herein can be administered in a single dose, or in several doses, for example daily, during a course of treatment. However, the therapeutically effective amount can depend on the subject being treated, the severity and type of the condition being treated, and the manner of administration. A unit dosage form of the agent can be packaged in a therapeutic amount, or in multiples of the therapeutic amount, for example, in a vial (e.g., with a pierceable lid) or syringe having sterile components.

Fibroblast growth factor 2 (FGF-2): (e.g., OMIM 134920) Also known as basic fibroblast growth factor (bFGF) and FGF-β. A growth factor and signaling protein that binds to and exerts effects via specific fibroblast growth factor receptor (FGFR) proteins, a family of closely related molecules. FGF-2 is involved in cellular proliferation, wound healing and angiogenesis. Thus, FGF-2 affibodies can be used to control the release of FGF-2 and increase angiogenesis, for example to treat a wound or vascular disease. Exemplary FGF-2 sequences can be found in the GenBank® database (e.g., Accession Nos. NP_001997.5, NM_002006.6, NP_001348594.1, and NP_032032.1). In some examples, an FGF-2 protein or coding sequence has at least 80%, at least 90%, at least 95%, at least 96%, at least 97%, at least 98%, at least 99% or 100% sequence identity to the sequence provided in NP_001997.5, NM_002006.6, NP_001348594.1, or NP_032032.1.

Glial derived neurotrophic factor (GDNF): (e.g., OMIM 600837) A protein that promotes survival of neurons. Thus, GDNF affibodies can be used to control the release of GDNF and increase survival of neurons, or promote the proliferation of new neurons, for example to treat a neurological disorder or injury, such as stroke, spinal cord injury, and traumatic brain injury. Exemplary GDNF sequences can be found in the GenBank® database (e.g., Accession Nos. ABU49429.1, nt 562-1197 of NM_000514.4, NP_001288261.1 and aa 78 to 211 of NP_000505.1 or AAI28109.1). In some examples, a GDNF protein or coding sequence has at least 80%, at least 90%, at least 95%, at least 96%, at least 97%, at least 98%, at least 99% or 100% sequence identity to the sequence provided in ABU49429.1, nt 562-1197 of NM_000514.4, NP_001288261.1, or aa 78 to 211 of NP_000505.1 or AAI28109.1.

Granulocyte-macrophage colony-stimulating factor (GM-CSF): (e.g., OMIM 138960) A monomeric glycoprotein secreted by macrophages, T cells, mast cells, natural killer cells, endothelial cells and fibroblasts that functions as a cytokine. The pharmaceutical analogs of naturally occurring GM-CSF are called sargramostim and molgramostim. GM-CSF facilitates myeloid stem cell differentiation and can be supplemented at an injury site to increase the efficacy of tissue repair. The immune functions of GM-CSF depend on its targeted presentation during the inflammatory stage of the regenerative cascade, but current protein delivery methods rely on administering supraphysiological doses that act over short periods of time and may cause off-target effects. Thus, GM-CSF affibodies can be used to control the release of GM-CSF and manipulate the immune response or increase angiogenesis, for example to treat a wound or vascular disease. Exemplary GM-CSF sequences can be found in the GenBank® database (e.g., Accession Nos. NP_000749.2; NP_446304.1, NP_999283.1, and M13207.1). In some examples, a GM-CSF protein or coding sequence has at least 80%, at least 90%, at least 95%, at least 96%, at least 97%, at least 98%, at least 99% or 100% sequence identity to the sequence provided in NP_000749.2; NP_446304.1, NP_999283.1, or M13207.1.

Hydrogel: A three-dimensional crosslinked hydrophilic polymer. Hydrogels include a mixture of porous, permeable polymers and at least 10% by weight or volume of interstitial fluid (e.g., water). They can be highly absorbent yet maintain well defined structures. Hydrogels can be prepared using polymeric materials, including hyaluronic acid, polyethylene glycol, collagen, and gelatin. The hydrogels provided herein include reversible and non-reversible covalent cross-linking bonds and include one or more affibodies and their corresponding protein. Such hydrogels can include other components. In some examples, a hydrogel is sterile.

Interleukin 4 (IL-4): (e.g., OMIM 147780) A cytokine that induces differentiation of naive helper T cells (Th0 cells) to Th2 cells. Upon activation by IL-4, Th2 cells subsequently produce additional IL-4 in a positive feedback loop. IL-4 is produced primarily by mast cells, Th2 cells, eosinophils and basophils. Thus, IL-4 affibodies can be used to control the release of IL-4 and regulate the immune system, for example reduce inflammation to treat a wound. Exemplary IL-4 sequences can be found in the GenBank® database (e.g., Accession Nos. CAP72493.1, AM937235.1, AAH27514.1 and AAA31055.1). In some examples, an IL-4 protein or coding sequence has at least 80%, at least 90%, at least 95%, at least 96%, at least 97%, at least 98%, at least 99% or 100% sequence identity to the sequence provided in CAP72493.1, AM937235.1, AAH27514.1 or AAA31055.1.

Isolated: An “isolated” biological component, such as a nucleic acid, protein (including affibodies) or organelle, has been substantially separated or purified away from other biological components in the environment (such as a cell) in which the component occurs, for example other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins.

Platelet-derived growth factor (PDGF): Growth factors that regulate cell growth and division. PDGF plays a significant role in blood vessel formation, the growth of blood vessels from already-existing blood vessel tissue, mitogenesis, e.g., proliferation, of mesenchymal cells such as fibroblasts, osteoblasts, tenocytes, vascular smooth muscle cells and mesenchymal stem cells as well as chemotaxis, the directed migration, of mesenchymal cells. Platelet-derived growth factor is a dimeric glycoprotein that can be composed of two A subunits (PDGF-AA), two B subunits (PDGF-BB), or one of each (PDGF-AB). In one example PDGF is PDFG-BB (e.g., OMIM 190040). Thus, PDGF affibodies can be used to control the release of PDGF and increase angiogenesis, for example to treat a wound. Exemplary PDGF-BB sequences can be found in the GenBank® database (e.g., Accession Nos. CAA45383.1, X63966.1, and SM94286.1). In some examples, a PDGF-BB protein or coding sequence has at least 80%, at least 90%, at least 95%, at least 96%, at least 97%, at least 98%, at least 99% or 100% sequence identity to the sequence provided in CAA45383.1, X63966.1, or SM94286.1.

Pharmaceutically acceptable carriers: The pharmaceutically acceptable carriers of use are conventional. Remington's Pharmaceutical Sciences, by E. W. Martin, Mack Publishing Co., Easton, PA, 19th Edition, 1995, describes compositions and formulations suitable for pharmaceutical delivery of the disclosed hydrogels.

Exemplary pharmaceutically and physiologically acceptable fluids includes water, physiological saline, balanced salt solutions, aqueous dextrose, glycerol or the like. In particular embodiments, suitable for administration to a subject the carrier may be sterile, and/or suspended or otherwise contained in a unit dosage form containing one or more measured doses of the composition suitable to induce the desired response. The unit dosage form may be, for example, in a sealed vial that contains sterile contents or a syringe for injection into a subject.

Peptide or Polypeptide: A polymer in which the monomers are amino acid residues that are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to those that are recombinantly or synthetically produced. A peptide has an amino (N) terminus and a carboxy (C) terminus. The N- or C-terminus of a polypeptide can be joined (for example, by peptide bond) to heterologous amino acids, such as a peptide tag, or a cysteine (or other, such as Lys, Tyr, Try, or Phe) residue in the context of a linker for conjugation chemistry.

The phrase “functional fragment(s) of a polypeptide” refers to all fragments of a polypeptide that retain an activity, or a measurable portion of an activity, of the polypeptide from which the fragment is derived.

Purified: The term purified does not require absolute purity; rather, it is intended as a relative term. Thus, for example, a purified affibody preparation is one in which the affibody is more enriched than the affibody is in its environment within a cell or other mixture. In one aspect, a preparation is purified such that the affibody represents at least 50% of the total protein content of the preparation. Substantial purification denotes purification from other proteins or cellular components. A substantially purified affibody is at least 60%, 70%, 80%, 90%, 95%, 98%, 99%, 99.9% or 99/99% pure. Thus, in one specific, non-limiting example, a substantially purified affibody is 90% free of other proteins or cellular components.

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide or nucleic acid molecule will possess a relatively high degree of sequence identity when aligned using standard methods.

Methods of alignment of sequences for comparison are known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Variants of an affibody provided herein are typically characterized by possession of at least about 80%, for example at least about 85%, 90%, 95%, 96%, 97%, 98% or 99% sequence identity counted over the full-length alignment with the amino acid sequence of the affibody using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Affibodies with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10⁻²⁰ amino acids and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that variants with similar activity could be obtained that fall outside of the ranges provided.

Subject or patient: A term that includes human and non-human mammals. In one example, the subject is a human or veterinary subject, such as a mouse, rat, dog, cat, or non-human primate. In some examples, the subject is a mammal (such as a human) who has a bone injury (such as a fracture, such as a non-union fracture, or due to cancer, osteoporosis, or osteoarthritis), wound (including wounds that damage vascular networks), a vascular disease (e.g., diabetic ulcer, critical limb ischemia, peripheral artery disease, cerebrovascular diseases including stroke, migraine and other headache disorders), or neurological injury or disorder (e.g., paralysis, acute spinal cord injury, stroke, traumatic brain injury, other head trauma, epilepsy, Alzheimer's disease and other dementias, ALS, multiple sclerosis, Parkinson's disease).

Synthetic: Produced by artificial means in a laboratory, for example a synthetic nucleic acid or protein (for example, an affibody) can be chemically synthesized in a laboratory.

Treating a disease: Includes inhibiting or preventing the partial or full development or progression of a disease, for example in a person who is known to have a predisposition to a disease. Furthermore, treating a disease refers to a therapeutic intervention that ameliorates at least one sign or symptom of a disease or pathological condition, or interferes with a pathophysiological process, after the disease or pathological condition has begun to develop.

Under conditions sufficient for: A phrase that is used to describe any environment that permits the desired activity. In one example, includes administering a therapeutically effective amount of a hydrogel composition as provided herein sufficient to enable the desired activity.

Vasculature: The network of blood vessels connecting the heart with all other organs and tissues in the body. It includes the arteries and arterioles, bringing oxygen-rich blood to the organs and tissues, and the veins and venules carrying deoxygenated blood back to the heart. A “resistance artery” is a blood vessel in the microcirculation that contributes to the creation of resistance to blood flow. Resistance vessels are innervated by autonomic nerves, and constrict and dilate in response to circulating hormones. Resistance in small arteries (lumen diameter<350 micrometers) and arterioles (lumen diameter<100 micrometers) accounts for 45-50% of total peripheral resistance.

Vascular endothelial growth factor (VEGF): A signal protein produced by many cells that stimulates the formation of blood vessels. VEGF is a sub-family of growth factors, the platelet-derived growth factor family of cystine-knot growth factors. They are signaling proteins involved in vasculogenesis (the de novo formation of the embryonic circulatory system) and angiogenesis (the growth of blood vessels from pre-existing vasculature). In one example VEGF is VEGF₁₆₅ (also known as neuropilin, e.g., OMIM 602069), a transmembrane protein involved in vasculogenesis and angiogenesis. Thus, VEGF affibodies can be used to control the release of VEGF and increase angiogenesis, for example to treat a wound or vascular disease. Exemplary VEGF₁₆₅ sequences can be found in the GenBank® database (e.g., Accession Nos. AAC12921, AAC51759.1, AF016050.1, AAC53345.1 and BAA08789.1). In some examples, a VEGF₁₆₅ protein or coding sequence has at least 80%, at least 90%, at least 95%, at least 96%, at least 97%, at least 98%, at least 99% or 100% sequence identity to the sequence provided in GenBank Accession No. AAC12921, AAC51759.1, AF016050.1, AAC53345.1 or BAA08789.1.

Wound: An injury or damage to living tissue.

Wound repair: The process of replacing damaged or missing cellular structures or tissue layers. Wound repair (or wound healing) is characterized by the steps of hemostasis (blood clotting), inflammation, proliferation (growth of new tissues) and remodeling.

### Overview

Directed evolution was used to generate affibodies, which are a class of small, α-helical, antibody-mimetic proteins that can be engineered to bind to a target protein of interest.^{42,43} Affibodies are currently being tested clinically and preclinically as targeting agents for HER2⁺ breast cancer cells,^{44,45} and for the detection of other biological markers, such as CD69 cell markers for early detection of activated immune cells⁴⁶ and vascular endothelial growth factor receptor-2 (VEGFR2) expression for analyzing angiogenesis signaling pathways.⁴⁷ Moreover, affibodies have also been used to tune the release of fibroblast growth factor-2 (FGF-2),⁴⁰ insulin-like growth factor-1 (IGF-1), and pigment epithelium-derived factor (PEDF).³⁹ Their clinical benefit is derived from their relatively stable structure under physiological conditions, the diversity of proteins to which they can bind, and the ability to modify their binding affinity by changing 13 to 17 amino acids at the binding interface between the affibody and target protein.^{43,48} However, the tunability of affibody affinity is underutilized, as affibody affinity has thus far only been maximized for targeting endogenous protein species without considering the use of multiple affibodies displaying a range of moderate affinities for tuning the delivery rates of exogenous proteins. While typical affinity binders generated via directed evolution target strong interactions with equilibrium dissociation constants in the picomolar range,⁴⁹ affibodies with moderate affinity interactions with equilibrium dissociation constants in the nanomolar range enable controlled protein release. Hydrogel delivery vehicles that include affibodies with different affinities for a protein of interest can be tuned to release proteins at specific rates.

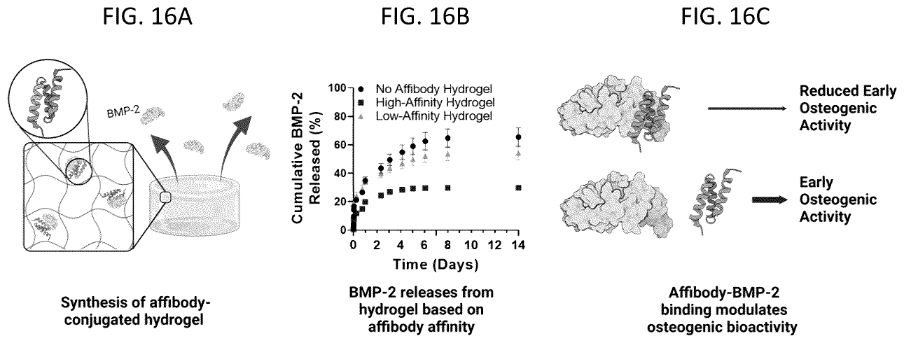

It is shown herein that BMP-2-specific affibodies were identified with a range of affinities for BMP-2 from a yeast surface display library containing 10⁸ affibody variants and used these affibodies to tune BMP-2 release from a polyethylene glycol-maleimide (PEG-Mal) hydrogel that could be prepared and used in a similar manner to the clinically used implantable collagen sponge. BMP-2-specific affibodies were identified that minimally interact with other proteins involved in the tissue healing cascade and have significantly different equilibrium dissociation constants to tune the release kinetics of BMP-2. In some examples, these BMP-2-specific affibodies did not interact with several other key proteins in the bone healing cascade: vascular endothelial growth factor (VEGF), interleukin-4 (IL-4), or granulocyte macrophage colony stimulating factor (GM-CSF). Computational modeling was used to predict the binding interface between the affibodies and BMP-2, revealing that the high-affinity binder may bind BMP-2 at a different interface than the low-affinity binder. BMP-2 bound to affibodies demonstrated diminished osteogenic properties in vitro. The integration of the affibodies into PEG-Mal hydrogels slowed the release of BMP-2, with the high-affinity affibody reducing BMP-2 release to a greater extent than the low-affinity affibody (FIGS. 16A-16B). Furthermore, all hydrogels released bioactive BMP-2 for seven days that induced ALP activity in C2C12 cells.

In addition to BMP-2 affibodies, using similar methods, affibodies for vascular endothelial growth factor (VEGF), fibroblast growth factor 2 (FGF-2), platelet-derived growth factor (PDGF), granulocyte-macrophage colony-stimulating factor (GM-CSF), inteleukin-4 (IL-4), and glial derived neurotrophic factor (GDNF) were identified and tested.

These findings demonstrate the use of affibodies in hydrogels for controlling protein bioactivity and release. The computational modeling results identify where on the protein an affibody may bind to allow for control of the activity of a protein (FIG. 16C). The ability to identify affibodies that impact protein bioactivity permits spatiotemporal control over protein activity. Unlike other affinity-based delivery systems that rely on protein-material interactions that may be nonspecific and unpredictable in in vivo-mimicking environments, the affibodies disclosed herein provide the specificity necessary to tune protein release in vivo more precisely, which can improve clinical protein delivery strategies.

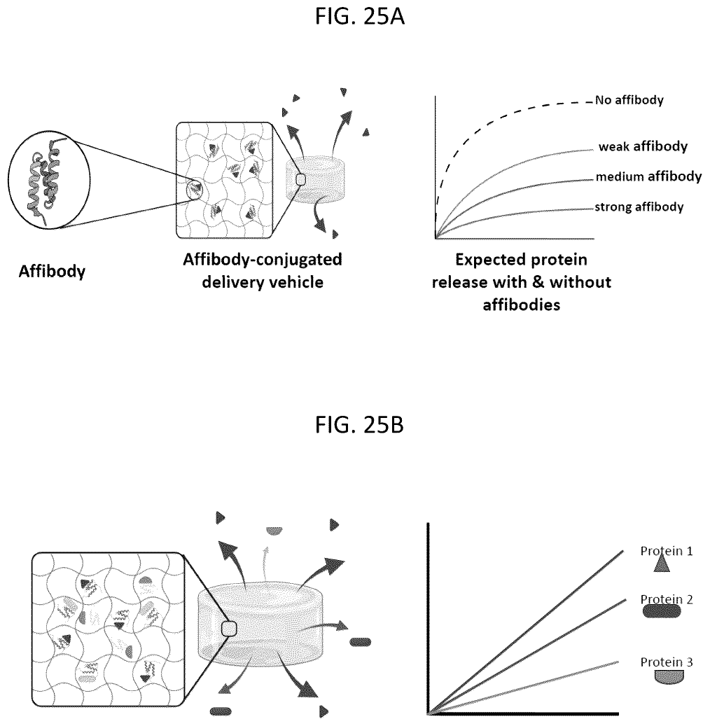

An overview of the technology is provided in FIGS. 25A and 25B. As shown in FIG. 25A, a hydrogel-affibody composition can include one or more affibodies specific for a single protein, wherein each unique affibody has a particular K_{D}. The hydrogel includes the affibodies bound to their corresponding protein. Due to the differences in K_{D}'s, the rate of release of the protein from the hydrogel will vary depending on the K_{D} of the affibody. For example, as shown in the graph, a weak affinity affibody (e.g., one with a higher K_{D}) will release its protein from the hydrogel more readily than a medium- or strong affinity affibody (e.g., one with a lower K_{D}). In some examples, the K_{D} of a high affinity affibody is at least 2-fold, at least 3-fold, at least 4-fold, at least 5-fold, at least 6-fold, at least 7-fold, at least 8-fold, at least 9-fold, or at least 10-fold lower than the K_{D} of a moderate affinity affibody or a low affinity affibody. In some examples, the K_{D} of a low affinity affibody is at least 2-fold, at least 3-fold, at least 4-fold, at least 5-fold, at least 6-fold, at least 7-fold, at least 8-fold, at least 9-fold, or at least 10-fold higher than the K_{D} of a moderate affinity affibody or a high affinity affibody. In some examples, the K_{D} of a moderate affinity affibody is at least 2-fold, at least 3-fold, at least 4-fold, at least 5-fold, at least 6-fold, at least 7-fold, at least 8-fold, at least 9-fold, or at least 10-fold higher than the K_{D} of a high affinity affibody or at least 2-fold, at least 3-fold, at least 4-fold, at least 5-fold, at least 6-fold, at least 7-fold, at least 8-fold, at least 9-fold, or at least 10-fold lower than the K_{D} of a low affinity affibody.

As shown in FIG. 25B, a hydrogel-affibody composition can include one or more affibodies specific for different proteins, wherein each unique affibody is specific for a particular protein. Three exemplary proteins are illustrated. The hydrogel includes the affibodies bound to their corresponding protein. Due to the differences in K_{D}'s of each unique affibody, the rate of release of the proteins from the hydrogel will vary depending on the K_{D} of the affibody. For example, as shown in the graph, a weak affinity affibody (binds triangle protein) will release its protein from the hydrogel more readily than a medium—(binds rod protein) or strong affinity affibody (binds half-circle protein).

One skilled in the art will appreciate that a hydrogel can include (a) one or more affibodies specific for one protein, wherein each unique affibody has a specific K_{D} for the protein, or (b) one or more affibodies specific for one or more proteins, wherein each unique affibody has a specific K_{D} for its corresponding protein.

### Compositions

The present disclosure provides compositions that include a hydrogel, one or more proteins, and one or more affibodies specific for the one or more proteins, wherein the protein and antibodies are covalently conjugated to (referred to herein as a hydrogel-affibody composition). The affibodies and proteins can be incorporated within the hydrogel. In some examples, the one or more proteins are non-covalently bound to the one or more affibodies in the hydrogel. For example, if the affibody is specific for BMP-2, the hydrogel can include BMP-2 bound to one or more different BMP-2-specific affibodies. Such compositions can further include a pharmaceutically acceptable carrier, such as water or saline or a buffer. In some examples, such compositions can be used to control the release of proteins in the hydrogel, which are bound to the affibodies.

In some examples, the hydrogel-affibody composition includes at least one of bone morphogenetic protein 2 (BMP-2) protein, vascular endothelial growth factor (VEGF) protein (such as VEGF₁₆₅), fibroblast growth factor 2 (FGF-2) protein, platelet-derived growth factor (PDGF) protein (such as PDGF-BB), granulocyte-macrophage colony-stimulating factor (GM-CSF) protein, inteleukin-4 (IL-4) protein, and glial derived neurotrophic factor (GDNF) protein, and corresponding affibodies specific for BMP-2, VEGF, FGF-2, PDGF, GM-CSF, IL-4, and/or GDNF.

In some examples, the hydrogel contains one or more unique affibodies specific for a single protein (such as one of BMP-2, VEGF, FGF-2, PDGF, GM-CSF, IL-4, or GDNF). In some examples, the hydrogel contains one unique affibody specific for a single protein (such as one of BMP-2, VEGF, FGF-2, PDGF, GM-CSF, IL-4, or GDNF). In some examples, the hydrogel contains at least 1 unique affibody (such as at least 2, at least 3, at least 4, at least 5 or at least 10, such as 2, 3, 4, 5, 6, 7, 8, 9, or 10 unique affibodies) specific for a single protein (such as one of f BMP-2, VEGF, FGF-2, PDGF, GM-CSF, IL-4, or GDNF), wherein each unique affibody has a different K_{D} for the protein. For example, the hydrogel can include a weak affinity affibody (e.g., one with a higher K_{D}), and a strong affinity affibody (e.g., one with a lower K_{D}) for BMP-2, VEGF, FGF-2, PDGF, GM-CSF, IL-4, or GDNF. In one example, the hydrogel includes a weak affinity affibody (e.g., one with a higher K_{D}), a medium affinity affibody (e.g., one with a K_{D} lower than that of the weak affinity antibody), and a strong affinity affibody (e.g., one with a K_{D} lower than the weak or medium-affinity affibody) for BMP-2, VEGF, FGF-2, PDGF, GM-CSF, IL-4, or GDNF.

In some examples, the hydrogel-affibody composition includes two or more proteins (or 3 or more, 4 or more, 5 or more, 6 or more, or all 7 proteins) selected from BMP-2, VEGF, FGF-2, PDGF, GM-CSF, IL-4, and GDNF, and corresponding affibodies. In some examples, the hydrogel-affibody composition includes two or more proteins (such as 2, 3, 4, 5, 6, or 7 proteins) selected from BMP-2, VEGF, FGF-2, PDGF, GM-CSF, IL-4, and GDNF, and one corresponding unique affibody for each protein. In some examples, the hydrogel-affibody composition includes two or more proteins (such as 2, 3, 4, 5, 6, or 7 proteins) selected from BMP-2, VEGF, FGF-2, PDGF, GM-CSF, IL-4, and GDNF, and two or more corresponding unique affibodies for each protein (such as at least 3, at least 4, at least 5, or at least 10 unique affibodies for each protein, such as 2, 3, 4, or 5 unique affibodies for each protein). If 2 or more affibodies are present for the same protein, each unique affibody has a different K_{D} for the protein. For example, the hydrogel can include a weak affinity affibody (e.g., one with a higher K_{D}), and a strong affinity affibody (e.g., one with a lower K_{D}) for BMP-2, VEGF, FGF-2, PDGF, GM-CSF, IL-4, and/or GDNF. In one example, the hydrogel includes a weak affinity affibody (e.g., one with a higher K_{D}), a medium affinity affibody (e.g., one with a K_{D} lower than that of the weak affinity antibody), and a strong affinity affibody (e.g., one with a K_{D} lower than the weak or medium-affinity affibody) for BMP-2, VEGF, FGF-2, PDGF, GM-CSF, IL-4, and/or GDNF.

In some examples, the hydrogel-affibody composition includes the following proteins and one or more specific affibodies for the protein: a) VEGF, FGF2, and PDGF-BB; b) GM-CSF; c) GDNF; d) VEGF, FGF2, PDGF-BB, and BMP-2; e) GM-CSF and IL-4; f) GM-CSF, IL-4 and MCP-1; g) BMP-2 and IL-4; h) BMP-2; i) GM-CSF, IL-4, and BMP-2, or j) PDGF-BB and VEGF.

The hydrogels can include additional proteins and affibodies, such as collagen I, collagen III, and/or monocyte chemoattractant protein-1 (MCP-1), and one or more corresponding affibodies. In some examples the hydrogel-affibody composition further includes one or more additional chemoattractant proteins (e.g., MCP-1, SDF-1a) and affibodies, cytokine proteins (e.g., IL-10) and affibodies, immunomodulatory proteins (e.g., IL-10, MCP-1, G-CSF) and affibodies, and/or morphogen proteins (e.g., NGF, NT-3, BDNF) and affibodies.

The hydrogel-affibody composition can include at least 1, at least 2, at least 3, at least 4, at least 5, at least 10, at least 15, at least 20, at least 30, at least 40, or at least 50 (such as 1, 2, 3, 4, 5, 6, 7, 8, 9, 10, 15, 20, 25, 30, 35, 40, 45, 50, 75, 100 or more) different affibodies specific for a single protein. In such examples, each unique affibody can have a unique affinity or K_{D} for the protein, such as at least one with a low K_{D}/strong affinity (e.g., K_{D} about 10⁻⁹-10⁻⁸ M), at least one with a medium K_{D}/medium affinity (e.g., K_{D} about 10⁻⁷-10⁻⁶ M) and at least one with a higher K_{D}/weak affinity (e.g., K_{D} about 10⁻⁵-10⁻³ M). In some examples, each unique affibody has a unique affinity or K_{D} for the target protein, such as at least one with a low K_{D}/strong affinity (e.g., K_{D} about 10⁻⁹-10⁻⁸ M) and at least one with a higher K_{D}/weak affinity (e.g., K_{D} about 10⁻⁵-10⁻³ M). In some examples a low K_{D}/strong affinity affibody has a K_{D} that is at least 2-fold, at least 3-fold, at least 4-fold, at least 5-fold, at least 6-fold, at least 7-fold, at least 8-fold, at least 9-fold, or at least 10-fold greater than a medium K_{D}/medium affinity affibody. In some examples a high K_{D}/low affinity affibody has a K_{D} that is at least 2-fold, at least 3-fold, at least 4-fold, at least 5-fold, at least 6-fold, at least 7-fold, at least 8-fold, at least 9-fold, or at least 10-fold lower than a medium K_{D}/medium affinity affibody. In some examples, each unique affibody has a K_{D} for the protein that is at least an order of magnitude (e.g., at least about 10-fold) different from another unique affibody for the same protein. Thus, in some examples a low K_{D}/strong affinity affibody has a K_{D} that is at least about 10 times greater than a medium K_{D}/medium affinity affibody, and a medium K_{D}/medium affinity affibody has a K_{D} that is at least about 10 times greater than a higher K_{D}/weak affinity affibody.

In some examples, the hydrogel-affibody composition includes at least 1, at least 2, at least 3, at least 4, at least 5, at least 10, at least 15, at least 20, at least 30, at least 40, or at least 50 (such as 1, 2, 3, 4, 5, 6, 7, 8, 9, 10, 15, 20, 25, 30, 35, 40, 45, 50, 75, 100 or more) different/unique affibodies, wherein each unique affibody is specific for a single protein. In some examples, combinations are used (e.g., two or more affibodies specific for protein 1, and two or more affibodies specific for protein 2, etc.). In examples, where two or more unique affibodies are present that are specific for the same protein, each unique affibody can have a distinct K_{D}, such as one with a higher and another with a lower K_{D} (such as at least 2-fold, at least 3-fold, at least 5-fold, or at least 10-fold difference).

The hydrogel is three-dimensional crosslinked hydrophilic polymer that includes a mixture of porous, permeable polymers and at least 10% by weight or volume of interstitial fluid (e.g., water). In some examples, the hydrogel includes polymeric materials, such as hyaluronic acid (HA), polyethylene glycol (PEG), PEG-Maleimide, modified hyaluronic acid (e.g., Norbornene-HA, norbornene-oxidized-HA or oxidized-HA, hydrazide-HA, methacryalate-HA), thiolated poly(E-caprolactone) (PCL-SH), thiolated poly(lactide-co-glycolide) (PLGA-SH), thiolated silk-firbroin, modified gelatin (methacrylate (GelMA), oxidized gelatin, gelatin norbornene), collagen, or combinations thereof. In some examples, a hydrogel is sterile. To generate the hydrogel containing affibodies and corresponding proteins, the polymer is incubated with a solution containing affibodies and proteins under conditions that allow incorporation of the affibodies and proteins into the polymer. In some examples, hydrogels are formed by mixing two different modified polymers together with different functional groups at room temperature, under heating, and/or with stirring. In some examples, hydrogels are formed by mixing one modified polymer with a crosslinker with or without a free radical initiator and with or without heating and/or UV or visible light. The hydrogel is crosslinked through covalent, dynamic covalent (i.e., reversible), or electrostatic interactions. Affibodies are covalently conjugated to the polymer backbone of the hydrogel through a C-terminal amino acid on the C-terminus (such as Cys, Lys, Tyr, Try, or Phe) of the affibody and functional group on the polymer. In some examples, the C-terminal cysteine on the affibody is modified with another functional group to enable conjugated to a specific type of polymer. To maintain sterility for sterile hydrogels, the solutions can be sterile-filtered with a syringe filter and handled in a biosafety cabinet prior to mixing and crosslinking.

Exemplary affibody sequences encompassed by the disclosure are provided in Table 1, and can be used in the compositions and methods provided herein. IN some examples, the affibody sequences provided in Table 1 further include an additional C-terminal amino acid, such as Cys, Lys, Tyr, Try, or Phe, for example when present in a hydrogel. Thus, in some examples an affibody in a hydrogel-affibody composition provided herein comprises or consists of one or more of SEQ ID NOS: 1-74. In some examples an affibody in a hydrogel-affibody composition provided herein comprises or consists of one or more of SEQ ID NOS: 1-74 and further includes an additional C-terminal amino acid, such as Cys, Lys, Tyr, Try, or Phe (e.g., see Shadish and DeForest, Matter, 2:50-77, 2020, herein incorporated by reference in its entirety). In some examples, the isolated affibodies have at least 90%, at least 95%, at least 96%, at least 97%, at least 98%, at least 99%, or 100% sequence identity to SEQ ID NOS: 1-74, and in some examples further includes an additional C-terminal amino acid, such as Cys, Lys, Tyr, Try, or Phe. In some examples, the affibody consists of any one of SEQ ID NOS: 1-74. n some examples, the affibody consists of any one of SEQ ID NOS: 1-74 and an additional C-terminal amino acid, such as Cys, Lys, Tyr, Try, or Phe. In some examples, the affibody is 56-80 amino acids, such as 56-65, 57-58, or 56, 57, 58, 59, 60, 61, 62, 63, 64, 65, 66, 67, 68, 69 or 70 amino acids in length. In some examples, the affibody has 1, 2, 3, 4, 5 or 6 conservative amino acid substitutions. In one example, the one or more affibodies in a hydrogel-affibody composition include one or more of SEQ ID NOS: 1, 2, 3, 12, 13, 14, 20, 21, 22, 42, 43, 44, 57, 58, 59, 60, 61, 62, 63, and 64, wherein in some examples SEQ ID NO: 1, 2, 3, 12, 13, 14, 20, 21, 22, 42, 43, 44, 57, 58, 59, 60, 61, 62, 63, or 64 further includes an additional C-terminal amino acid, such as Cys, Lys, Tyr, Try, or Phe.

TABLE 1
Exemplary affibody sequences (SEQ ID NO: in parenthesis).

| | | K_{D} (nM) |
|---|---|---|
| BMP-2 Affibodies | | |
| High | AEAKYYKEVSS AATQIRYLPN LTAFQKAAFY | 10.7 |
| Affinity | AALLDDPSQS SELLSEAKKL NDSQAPK (1) | |
| (A1-2) | | |
| Moderate | AEAKYAKEQF NAYVVIFYLP NLTASQKAAF | 10.4 |
| Affinity | VDALSNDPSQ SSELLSEAKK LNDSQAPK (2) | |
| (A2-2) | | |
| Low | IVALFNDPSQ SSELLSEAKK LNDSQAPK (3) | 34.8 |
| Affinity | AEAKYYKEGD NAYNVIYGLP NLTRPQRLAF | |
| (B4-1) | | |
| A1-1 | AEAKYNKEVTAAANSIWVLPNLTGDQKAAFFEALLDDPS | |
| | QSSELLSEAKKLNDSQAPK (4) | |
| A1-3 | AEAKYTKEGFDAYDVIDNLPNLTLDQRNAFVYALENDPS | |
| | QSSELLSEAKKLNDSQAPK (5) | |
| A2-1 | AEAKYYKEWLDADMSIRSLPNLTGYQIRAFIAALGNDPS | |
| | QSSELLSEAKKLNDSQAPK (6) | |
| A2-3 | AEAKYYKERRAAAVVIFYLPNLTRVQKGAFIEALDDDPS | |
| | QSSELLSEAKKLNDSQAPK (7) | |
| A3-1 | AEAKYAKERLNAIYVINDLPNLTQGQRVAFARALYNDPS | |
| | QSSELLSEAKKLNDSQAPK (8) | |
| A3-2 | AEAKYAKEQFNAYVVIFYLPNLTASQKAAFVDALSNDPS | |
| | QSSELLSEAKKLNDSQAPK (9) | |
| B3-3 | AEAKYYKEWVNAYDQIRVLPNLTRFQRLAFYRALYNDPS | |
| | QSSELLSEAKKLNDSQAPK (10) | |
| B4-2 | AEAKYYKEWLDADMSIRSLPNLTGYQIRAFIAALGNDPS | |
| | QSSELLSEAKKLNDSQAPK (11) | |
| GM-CSF Affibodies | | |
| GM3-C4 | AEAKYTKELFNAVGEITALPNLTRYHLYAFYYALLNDPS | 441.4 |
| (High | QSSELLSEAKKLNDSQAPK (12) | |
| Affinity) | | |
| GM4-C4 | AEAKYNKEWFAADLSIGFLPNLTLDQLYAFVFALYDDPS | 971.0 |
| (Mid | QSSELLSEAKKLNDSQAPK (13) | |
| affinity) | | |
| GM4-C3 | AEAKYAKEGLNAYLSIRWLPNLTGDQMYAFISALLDDPS | 3783 |
| (Low | QSSELLSEAKKLNDSQAPK (14) | |
| Affinity) | | |
| GM1-C3 | AEAKYTKEGFNAYDEIDNLPNLTLDQRNAFVYALFNDPS | |
| | QSSELLSEAKKLNDSQAPK (15) | |
| GM2-C4 | AEAKYTKELFNAVGEITALPNLTRYHLYAFYYALLNDPS | |
| | QSSELLSEAKKLNDSQAPK (16) | |
| GM3-C1 | AEAKYNKEVGTANFEIVLLPNLTLYQMLAFIKALVNDPS | 296.7 |
| | QSSELLSEAKKLNDSQAPK (17) | |
| GM3-C2 | AEAKYNKEWYNAISVIFYLPNLTGFQRAAFVDALGDDPS | |
| | QSSELLSEAKKLNDSQAPK (18) | |
| GM4-C2 | AEAKYYKEGFYANFVIGALPNLTLVQRAAFYFALLNDPS | 786.7 |
| | QSSELLSEAKKLNDSQAPK (19) | |
| VEGF Affibodies | | |
| TM2 (High | AEAKYYKEGATAYRVIEYLPNLTGAQKAAFIDALYNDPS | 58.3 |
| Affinity) | QSSELLSEAKKLNDSQAPK (20) | |
| LG2 (Mid | AEAKYTKEGFDAYDVIDNLPNLTLDQRNAFVYALENDPS | 307 |
| Affinity) | QSSELLSEAKKLNDSQAPK (21) | |
| BR2 (Low | AEAKYNKEWYDAVFVIGSLPNLTEDQKDAFSDALVDDPS | 6470 |
| Affinity) | QSSELLSEAKKLNDSQAPK (22) | |
| L1 | AEAKYYKEWNAAYVVINGLPNLTRRQREAFVHALVDDPS | |
| | QSSELLSEAKKLNDSQAPK (23) | |
| L3 | AEAKYYKERYAANYSIWVLPNLTLLQRFAFFFALSNDPS | |
| | QSSELLSEAKKLNDSQAPK (24) | |
| L4 | AEAKYAKELDDAFFEIASLPNLTGFQLHAFAVALGNDPS | |
| | QSSELLSEAKKLNDSQAPK (25) | |
| L5 | AEAKYNKERDSAYSVIWGLPNLTDSQKAAFGYALYNDPS | |
| | QSSELLSEAKKLNDSQAPK (26) | |
| L7 | AEAKYAKELEAANMVIVDLPNLTHGQKVAFLVALENDPS | |
| | QSSELLSEAKKLNDSQAPK (27) | |
| L8 | AEAKYNKEWYDAILEIGFLPNLTGHQRDAFSDALVDDPS | |
| | QSSELLSEAKKLNDSQAPK (28) | |
| L10 | AEAKYNKEQDSAYSVIWGLPNLTESQKAAFGYALYDDPS | |
| | QSSELLSEAKKLNDSQAPK (29) | |
| BM1 | AEAKYNKEVTAAANSIWVLPNLTGDQKAAFFEALLDDPS | |
| | QSSELLSEAKKLNDSQAPK (30) | |
| BM2 | AEAKYAKEWFYAYHVIYDLPNLTGFQKHAFYLALYDDPS | |
| | QSSELLSEAKKLNDSQAPK (31) | |
| BM3 | AEAKYNKEVTAAANSIWVLPNLTGDQKAAFFEALLDDPS | |
| | QSSELLSEAKKLNDSQAPK (32) | |
| BM7 | AEAKYAKEGATAFGSIPYLPNLTDVQRYAFIVALLDDPS | |
| | QSSELLSEAKKLNDSQAPK (33) | |
| BR1 | AEAKYTKEWYAAVVQIGYLPNLTAFQRAAFSFALSNDPS | |
| | QSSELLSEAKKLNDSQAPK (34) | |
| BR3 | AEAKYTKERDDASLEIAYLPNLTPYQLMAFFFALSNDPS | |
| | QSSELLSEAKKLNDSQAPK (35) | |
| BR5 | AEAKYAKEWTNAFVSIVCLPNLTAVQREAFVLALVDDPS | |
| | QSSELLSEAKKLNDSQAPK (36) | |
| BR6 | AEAKYAKEWEDAINEIWCLPNLTEYQRIAFVSALYNDPS | |
| | QSSELLSEAKKLNDSQAPK (37) | |
| BR7 | AEAKYAKELLNAFDEIYGLPNLTVGQRMAFCDALINDPS | |
| | QSSELLSEAKKLNDSQAPK (38) | |
| TM4 | AEAKYYKEWYDAFVVIDALPNLTAYQREAFIFALVNDPS | |
| | QSSELLSEAKKLNDSQAPK (39) | |
| TM6 | AEAKYYKEWVDAYLVIDSLPNLTRLQVEAFVFALVNDPS | |
| | QSSELLSEAKKLNDSQAPK (40) | |
| TM7 | AEAKYTKEVDYAACVIAYLPNLTGVQVYAFYRALADDPS | |
| | QSSELLSEAKKLNDSQAPK (41) | |
| FGF-2 Affibodies | | |
| FG2-C1 | AEAKYTKEGSDAFDVIVLLPNLTRDQRDAFLYALLDDPS | 3.08 |
| (High) | QSSELLSEAKKLNDSQAPK (42) | |
| FG3-C1 | AEAKYAKEWLSADYVIICLPNLTLDQMVAFYDALENDPS | 121 |
| (Mid) | QSSELLSEAKKLNDSQAPK (43) | |
| FG3-C4 | AEAKYNKEVFDADCSIWYLPNLTRYQISAFQSALDDDPS | 4550 |
| (Low) | QSSELLSEAKKLNDSQAPK (44) | |
| FG1_C1 | AEAKYTKEGCDAYTEIVDLPNLTGYQRRAFYWALENDPS | |
| | QSSELLSEAKKLNDSQAPK (45) | |
| FG1_C2 | AEAKYNKEMPDANCQIAFLPNLTQYQVPAFIYALCNDPS | |
| | QSSELLSEAKKLNDSQAPK (46) | |
| FG1_C3 | AEAKYNKEGEDATTQIGSLPNLTQAQKHAFAVALGNDPS | |
| | QSSELLSEAKKLNDSQAPK (47) | |
| FG1_C4 | AEAKYSKEGFYADWVIPVLPNLTRKQRVAFHDALHNDPS | |
| | QSSELLSEAKKLNDSQAPK (48) | |
| FG2-C3 | AEAKYAKEWLDAIDVIGYLPNLTDFORGAFYDALNDDPS | |
| | QSSELLSEAKKLNDSQAPK (49) | |
| FG3_C2 | AEAKYYKEGYNAIVEIRCLPNLTDCQVAAFIDALDDDPS | |
| | QSSELLSEAKKLNDSQAPK (50) | |
| FG3-C3 | AEAKYAKELDAAYVVIYFLPNLTHCQMVAFLHALSDDPS | |
| | QSSELLSEAKKLNDSQAPK (51) | |
| FG4-C1 | AEAKYSKEVYSAYDVIFALPNLTQYQVLAFFDALCDDPS | |
| | QSSELLSEAKKLNDSQAPK (52) | |
| FG4-C2 | AEAKYAKERLTAVCSIVALPNLTEGQMVAFDDALHDDPS | |
| | QSSELLSEAKKLNDSQAPK (53) | |
| FG4-C3 | AEAKYAKEGFNAVNVIWPLPNLTADQVCAFICALADDPS | |
| | QSSELLSEAKKLNDSQAPK (54) | |
| FG4-C4 | AEAKYAKEGCTAFLEIAALPNLTGYQRDAFIEALFDDPS | |
| | QSSELLSEAKKLNDSQAPK (55) | |
| FG2-CA | AEAKYTKEGSDAFDVIVLLPNLTRDQRDAFLYALLDDPS | |
| | QSSELLSEAKKLNDSQAPK (56) | |
| PDGF Affibodies | | |
| BR6 | AEAKYYKEWDSASDSIGFLPNLTRAQMVAFFAALFNDPS | |
| | QSSELLSEAKKLNDSQAPK (57) | |
| 0010 | AEAKYAHELWEADWEITNLPNLSPDQLMAFYMALWDDPS | 1.5 |
| (High) | QSSELLSEAKKLNDSQAPK (58) | |
| 0057 | AEAKYAFELWEAQHEIQQLPNLRPDQIAAFAMALYDDPS | 5.5 |
| (High) | QSSELLSEAKKLNDSQAPK (59) | |
| BM_6 | AEAKYAKELDDASVEIWDLPNLTPCQKVAFFVALYDDPS | 855 |
| (Medium) | QSSELLSEAKKLNDSQAPK (60) | |
| IL-4 Affibodies | | |
| G3H-C3 | AEAKYNKELDAADADVEIWLLPNLTLDQLLAFIAALFND | 4 |
| | PSQSSELLSEAKKLNDSQAPK (61) | |
| G3H-C7 | AEAKYTKELSDANAEIWSLPNLTVDQLVAFIFALWDDPS | 92000 |
| | QSSELLSEAKKLNDSQAPK (62) | |
| G3H-C10 | AEAKYSKEQSNAYASITDLPNLTRLQKLAFWVALFNDPS | |
| | QSSELLSEAKKLNDSQAPK (63) | |
| AD_189 | AERKYHWELLVAFMEIQSLPNLTKDQITQFMAALEDDPS | |
| | QSSELLSEAKKLNDSQAPK (64) | |
| Glial Derived Neurotrophic | | |
| Factor (GDNF) Affibodies | | |
| A1 | AEAKYNKEQVYASDSIQVLPNLTATQRVAFDPALHNDPS | |
| | QSSELLSEAKKLNDSQAPK (65) | |
| A2 | AEAKYNKEKPNAVGEISVLPNLTEFQMVAFIFALVNDPS | |
| | QSSELLSEAKKLNDSQAPK (66) | |
| A3 | AEAKYAKEWTTANYSIGVLPNLTLTQRYAFETALFDDPS | |
| | QSSELLSEAKKLNDSQAPK (67) | |
| B4 | AEAKYTKERHDATLVIHVLPNLTDARILAFIVALSNDPS | |
| | QSSELLSEAKKLNDSQAPK (68) | |
| B6 | AEAKYNKERSNASFEILVLPNLTGIQKGAFFAALPDDPS | |
| | QSSELLSEAKKLNDSQAPK (69) | |
| B7 | AEAKYSKEWYDAYLVIFVLPNLTQFORPAFPPALKNDPS | |
| | QSSELLSEAKKLNDSQAPK (70) | |

In one example, provided are one or more of the BMP-2 affibodies of SEQ ID NOS: 1-11 or 71-73, which in some examples are present in a hydrogel. Such a hydrogel can further include BMP-2, and can be used to control release of BMP-2 from the hydrogel, for example in the treatment of a bone or cartilage injury (for example by applying the hydrogel to an injury site on bone or cartilage).

In one example, provided are one or more of the GM-CSF affibodies of SEQ ID NOS: 12-19 or 74, which in some examples are present in a hydrogel. Such a hydrogel can further include GM-CSF, and can be used to control release of GM-CSF from the hydrogel, for example in the treatment of a wound (for example by applying the hydrogel to a wound or injury site).

In one example, provided are one or more of the VEGF affibodies of SEQ ID NOS: 20-41, which in some examples are present in a hydrogel. Such a hydrogel can further include VEGF, and can be used to control release of VEGF from the hydrogel, for example to stimulate angiogenesis, for example in the treatment of a wound (for example by applying the hydrogel to a wound or injury site) or vascular disease.

In one example, provided are one or more of the FGF-2 affibodies of SEQ ID NOS: 42-56, which in some examples are present in a hydrogel. Such a hydrogel can further include FGF-2, and can be used to control release of FGF-2 from the hydrogel, for example in the treatment of a wound (for example by applying the hydrogel to a wound or injury site) or vascular disease.

In one example, provided are one or more of the PDGF affibodies of SEQ ID NOS: 57-60, which in some examples are present in a hydrogel. Such a hydrogel can further include PDGF, and can be used to control release of PDGF from the hydrogel, for example in the treatment of a wound (for example by applying the hydrogel to a wound or injury site) or vascular disease.

In one example, provided are one or more of the IL-4 affibodies of SEQ ID NOS: 61-64, which in some examples are present in a hydrogel. Such a hydrogel can further include IL-4, and can be used to control release of IL-4 from the hydrogel, for example in the treatment of a wound by manipulating the immune response to injury (for example by applying the hydrogel to a wound or injury site).

In one example, provided are one or more of the glial derived neurotrophic factor (GDNF) affibodies of SEQ ID NOS: 65-70, which in some examples are present in a hydrogel. Such a hydrogel can further include GDNF, and can be used to control release of GDNF from the hydrogel, for example in the treatment of a neurological disorder or injury (for example by applying the hydrogel to an injury site).

### Methods of Treatment

Methods of using the disclosed hydrogel-affibody compositions to treat a disease, by administering an effective amount of the composition to a subject in need thereof. In some examples, two or more different hydrogel-affibody compositions (such as 2, 3, 4, or 5 different hydrogel-affibody compositions) are used in a treatment. Such administration can be systemic or localized. In some examples, the hydrogel-affibody compositions are administered directly to an injury site, for example as part of a surgical procedure. In some examples, multiple administrations are performed. The subject treated can be a mammal, such as a human or veterinary subject. Exemplary diseases/injuries that can be treated are provided in Table 2, with the appropriate affibodies/proteins listed.

TABLE 2
Exemplary Treatments

| Disease/Injury | Exemplary Affibodies |
|---|---|
| Bone or cartilage (e.g., | BMP-2 |
| fracture, cancer, | GM-CSF |
| osteoporosis, | IL-4 |
| osteoarthritis). | BMP-2 + GM-CSF |
| | BMP-2 + IL-4 |
| | BMP-2 + GM-CSF + IL-4 |
| Wound, vascular disease | VEGF |
| (e.g., diabetic ulcer, | FGF-2 |
| atherosclerosis, peripheral | PDGF |
| artery disease (PAD), | GM-CSF |
| carotid artery disease, | IL-4 |
| coronary artery disease, | VEGF + FGF-2 |
| critical limb ischemia, | VEGF + PDGF |
| Raynaud's disease, stroke, | VEGF + FGF-2 + PDGF |
| and cerebrovascular | VEGF + FGF-2 + PDGF + GM-CSF |
| disease) | VEGF + FGF-2 + PDGF + IL-4 |
| | VEGF + FGF-2 + PDGF + GM-CSF + IL-4 |
| Neuron (e.g., stroke, spinal | GDNF |
| cord injury, traumatic brain | GDNF + GM-CSF |
| injury, paralysis, | GDNF + IL-4 |
| Parkinson's Disease, | GDNF + GM-CSF + IL-4 |
| Alzheimer's Disease, ALS) | |

In one example, the subject has a bone injury, and the method includes administering the composition to the site of injury or systemic administration, and the hydrogel-affibody composition includes one or more BMP-2 affibodies, one or more IL-4 affibodies, and/or one or more GM-CSF affibodies. Exemplary bone injuries include fractures (such as those caused by trauma), for example in the spinal column, vertebrae (such as the lumbar vertebra), femur, tibia, fibula, thoracic cage, rib, clavicle, humerus, radius, ulna, tarsal bone, ilium, cranium, carpal bone, or a bone of the face (such as a mandible, nasal, zygomatic, lacrimal, maxilla, or sphenoid bone). In one example, the bone injury results from loss of bone, for example due to surgery, cancer, osteoporosis, osteoarthritis or other disease or injury. In one example, a subject is administered a hydrogel-affibody composition that includes one or more BMP-2 affibodies (such as at least 2, or at least 3 unique affibodies, such as 1, 2, 3, 4, 5, 6, 7, 8, 9, 10, or 11 of SEQ ID NOS: 1-11 or an affibody having at least 90% or at least 95% sequence identity to 1, 2, 3, 4, 5, 6, 7, 8, 9, 10, or 11 of SEQ ID NOS: 1-11). In one example, a subject is administered a hydrogel-affibody composition that includes one or more IL-4 affibodies (such as at least 2, or at least 3 unique affibodies, such as 1, 2, 3, or 4 of SEQ ID NOS: 61-64 or an affibody having at least 90% or at least 95% sequence identity to 1, 2, 3, or 4 of SEQ ID NOS: 61-64). In one example, a subject is administered a hydrogel-affibody composition that includes one or more GM-CSF affibodies (such as at least 2, or at least 3 unique affibodies, such as 1, 2, 3, 4, 5, 6, 7, or 8 of SEQ ID NOS: 12-19 or an affibody having at least 90% or at least 95% sequence identity to 1, 2, 3, 4, 5, 6, 7, or 8 of SEQ ID NOS: 12-19). In some examples, the hydrogel-affibody composition includes combinations of these affibodies.

In one example, the subject has an injury or disease that would benefit from increase angiogenesis, and the method includes administering a hydrogel-affibody composition to the site of injury or systemic administration, and the hydrogel-affibody composition includes one or more VEGF affibodies, one or more PDGF affibodies, one or more GM-CSF affibodies, and/or one or more FGF-2 affibodies. The hydrogel-affibody composition can control the release of such proteins. Angiogenesis, the process through which new blood vessels form, is a component of musculoskeletal healing, as it enables the transport of biomolecules to an injury site. Angiogenesis is mediated by a signaling cascade of key proteins; however, the temporal presentation of these proteins may be disrupted by factors such as age, severe injury severity, and chronic disease. Supplementation of angiogenic proteins, including VEGF, FGF-2, GM-CSF, and PDGF, using the hydrogel-affibody compositions provided herein, provides a method to stimulate angiogenesis. In one example, increased angiogenesis is used to treat a wound, such as one on the skin. Exemplary wound that can be treated include penetrating wounds, thermal burn, chemical burn, electric burn, surgical wound, puncture wounds, lacerations, abrasions, skin tears and diabetic ulcers. In one example, increased angiogenesis is used to treat a vascular disease, such as a disease of the arteries, veins, capillaries, and lymph vessels. Exemplary vascular diseases that can be treated include atherosclerosis, peripheral artery disease (PAD), carotid artery disease, coronary artery disease, critical limb ischemia, Raynaud's disease, stroke, and cerebrovascular disease. In one example, the subject with a wound and/or a vascular disease is diabetic.

In one example, a subject with a wound or vascular disease is administered a hydrogel-affibody composition that includes one or more GM-CSF affibodies (such as at least 2, or at least 3 unique affibodies, such as 1, 2, 3, 4, 5, 6, 7, or 8 of SEQ ID NOS: 12-19 or an affibody having at least 90% or at least 95% sequence identity to 1, 2, 3, 4, 5, 6, 7, or 8 of SEQ ID NOS: 12-19). In one example, a subject with a wound or vascular disease is administered a hydrogel-affibody composition that includes one or more VEGF affibodies (such as at least 2, or at least 3 unique affibodies, such as 1, 2, 3, 4, 5, 6, 7, 8, 9, 10, 11, 12, 13, 14, 15, 16, 17, 18, 19, 20, 21 or 22 of SEQ ID NOS: 20-41 or an affibody having at least 90% or at least 95% sequence identity to 1, 2, 3, 4, 5, 6, 7, 8, 9, 10, 11, 12, 13, 14, 15, 16, 17, 18, 19, 20, 21 or 22 of SEQ ID NOS: 20-41). In one example, a subject with a wound or vascular disease is administered a hydrogel-affibody composition that includes one or more FGF-2 affibodies (such as at least 2, or at least 3 unique affibodies, such as 1, 2, 3, 4, 5, 6, 7, 8, 9, 10, 11, 12, 13, 14, or 15 of SEQ ID NOS: 42-56 or an affibody having at least 90% or at least 95% sequence identity to, 2, 3, 4, 5, 6, 7, 8, 9, 10, 11, 12, 13, 14, or 15 of SEQ ID NOS: 42-56). In one example, a subject with a wound or vascular disease is administered a hydrogel-affibody composition that includes one or more PDGF affibodies (such as at least 2, or at least 3 unique affibodies, such as 1, 2, 3, or 4 of SEQ ID NOS: 57-60 or an affibody having at least 90% or at least 95% sequence identity to 1, 2, 3, or 4 of SEQ ID NOS: 57-60). In some examples, the hydrogel-affibody composition includes combinations of these affibodies.

In one example, a subject with a neurological disease or injury is administered a hydrogel-affibody composition that includes one or more GDNF affibodies (such as at least 2, or at least 3 unique affibodies, such as 1, 2, 3, 4, 5, or 6 of SEQ ID NOS: 65-70 or an affibody having at least 90% or at least 95% sequence identity to 1, 2, 3, 4, 5, or 6 of SEQ ID NOS: 65-70). Exemplary neurological diseases that can be treated include Parkinson's disease, Alzheimer's Disease, ALS, and epilepsy. Exemplary neurological disease injuries that can be treated include traumatic brain injury, traumatic spine injury, traumatic nerve injury, paralysis, and stroke.

### Example 1: Materials and Methods

This example provides the materials and methods used to generate the results described in Examples 2-12.

### Protein Modifications

Recombinant human BMP-2 (Medtronic, R&D Systems) was biotinylated using EZ-Link™ Sulfo-NHS-Biotin (Thermo Fisher) per the manufacturer's protocols. Briefly, a 10 mM solution of sulfo-NHS-biotin in water was prepared, and 20 molar excess of sulfo-NHS-biotin was added to a 0.5 mg mL⁻¹ solution of BMP-2 (Medtronic) in phosphate buffered saline (Fisher Scientific; PBS). The reaction was carried out for 2 hours at 4° C., and the biotinylated product (bBMP-2) was eluted into PBS using 7 kDa Zeba Spin Desalting Column (Thermo Fisher)). Biotinylation was confirmed using a Pierce™ Biotin Quantitation Kit (Thermo Fisher)).

### Yeast Growth and Induction

The naïve affibody-expressing yeast surface display library used was donated by Dr. Benjamin Hackel. This EBY100 strain of S. cerevisiae contains the pCT surface display vector for galactose-inducible surface protein expression of roughly 4×10⁸ unique affibody sequences.⁴³ Yeast were grown in selective growth media (16.8 g sodium citrate dihydrate, 3.9 g citric acid, 20.0 g dextrose, 6.7 g yeast nitrogen base, 5.0 g casamino acids, 1 mg ciprofloxacin and 100 mg ampicillin in 1 L reverse osmosis (RO) water) in an Innova44 shaking incubator (Innova) at 37° C. for 20 hours to a concentration between 5-10×10⁷ cells mL⁻¹, after which 10× library diversity was transferred into selective induction media (10.2 g sodium phosphate dibasic heptahydrate, 8.6 g sodium phosphate monobasic monohydrate, 19.0 g galactose, 1.0 g dextrose, 6.7 g yeast nitrogen base, 5.0 g casamino acids, 1 mg ciprofloxacin and 100 mg ampicillin in 1 L RO water) to induce affibody expression in a shaking incubator at 37° C. for 20 hours.

Surface protein expression was confirmed by flow cytometry. 1×10⁶ cells were aliquoted into tubes labeled cells only, secondary only, and c-myc+secondary. Each tube was washed and resuspended in 50 μL of PBS+0.1% BSA (PBSA). 1.25 μL of anti-c-myc mouse monoclonal antibody (αCMYC, 9E10; BioLegend) were added to the c-myc+secondary tube. The tubes were rotated at 4° C. for 30 minutes. All tubes were washed again and resuspended in 50 μL of PBSA. 0.625 μL of goat anti-mouse IgG-AlexaFluor™ 488 secondary antibody (Thermo Fisher; AF488) were added to the secondary only and c-myc+secondary tubes. All tubes were rotated for 30 minutes in the dark at 4° C. All tubes were washed twice and resuspended in 200 μL PBSA. Flow cytometry was performed using an Accuri™ C6 Plus Flow Cytometer with 96-well plate autosampler (Becton Dickinson).

### Magnetic Activated Cell Sorting

Magnetic-activated cell sorting (MACS) was performed to enrich for BMP-2-binding affibodies within the yeast surface display library. One round of MACS consisted of two negative bead sorts and one positive bead sort. Negative bead sorts were performed using carboxylic acid magnetic beads (COOH beads) conjugated with either tris or BSA, which removed non-specific binders.^{39,40,50,51} The positive bead sorts consisted of COOH beads conjugated with BMP-2 to enrich for yeast displaying BMP-2-specific affibodies. To prepare the beads, 2 μL of COOH beads (Invitrogen™ Dynabeads™ M-270 Carboxylic Acid) were rotated with 100 μL of cold 0.05 M NaOH for 10 minutes and then exposed to a magnetic field for 2 minutes so that a magnetic bead pellet formed at the wall of the tube. The NaOH was carefully removed to avoid disturbing the pellet, and the beads were then resuspended in 100 μL of cold water and rotated for 10 minutes. The beads were then resuspended and rotated in 100 μL of 50 mg mL⁻¹ solution of 1-Ethyl-3-(3-dimethylaminopropyl)carbodiimide (EDC) in water for 30 minutes. The EDC solution was removed, and the beads quickly rinsed with cold water and resuspended in 100 μL of 0.1 M MES buffer pH 5 followed by either 500 μL of PBSA, 500 μL of 0.05 M tris pH 7.4, or 33 pmol of carrier-free BMP-2 (R&D Biosystems) in water and rotated for 30 minutes. The reaction was terminated using the 0.05 M tris pH 7.4, and the beads were washed and resuspended in a solution of PBSA and stored on ice until needed.

10× library diversity was washed in PBSA to remove the induction media and resuspended in BSA-conjugated COOH bead solution. The yeast and beads were rotated at 4° C. for 2 hours and then exposed to a magnetic field. The unbound solution was gently removed and transferred to a tube containing tris-conjugated magnetic beads. The rotation and exposure were repeated as above, and the unbound solution was transferred to a tube with the BMP-2-conjugated magnetic beads and rotated once again for 2 hours. After exposure to the magnetic field, the unbound solution was removed, and the magnetic beads were resuspended in PBSA. 10 μL of 100× and 2000× diluted BSA, tris, and BMP-2-conjugated beads were plated on selective growth plates (16.8 g sodium citrate dihydrate, 3.9 g citric acid, 16 g bacto agar, 20 g dextrose, 6.7 g yeast nitrogen base, 5 g casamino acids, RO water, autoclaved and poured into petri dishes). Plates were incubated at 30° C. for 36 h, and colonies were counted to determine the ratio of positive-to-negative binders and new library diversity. The updated library diversity was estimated by the formula below, and the new diversity was used to determine the number of yeasts used for subsequent sorts.

$$library diversity = ( \frac{CFU}{plate} ) * ( dilution fac tor ) * ( \frac{total volume of undiluted positive sort}{10 μ L} )$$

### Fluorescence-Activated Cell Sorting

Fluorescence-activated cell sorting (FACS) was performed on the enriched yeast library after MACS to separate yeast into populations corresponding to approximately different affinity ranges for BMP-2 binding. 40×10⁶ induced yeast cells were aliquoted into tubes labeled cells only, secondary only, c-myc, and c-myc+bBMP-2, and washed in PBSA. The cells only, secondary only and c-myc tubes were resuspended in 50 μL PBSA. The c-myc+bBMP-2 tube was resuspended in 50 μL of 1 μM bBMP-2 in PBSA. 1.25 μL of αCMYC were also added to the c-myc and c-myc+bBMP-2 tubes. All tubes were rotated at 4° C. for 1 h and then washed with PBSA. Except for the cells-only control, all tubes were incubated with 50 μL of secondary fluorescent solution (10.4 μL of 333 nM goat anti-mouse IgG AlexaFluor™ 647, 3.25 μL of AlexaFluor™ 488 streptavidin conjugate, 187 μL PBSA). The tubes were all rotated at 4° C. for 30 minutes and washed 2 times in 500 μL of PBSA. The yeast was suspended in 1000 μL PBSA and sorted by a SH800 Cell Sorter (Sony Biotechnology). At least 10,000 cells were obtained from each gate. Following FACS, yeast from each collected gate were grown in selective growth media at 30° C. to an approximate concentration of 10⁷ cells mL⁻¹, plated onto selective growth plates, and incubated for 24-36 hours in 30° C.

### Gene Sequencing of Monoclonal Affibody Yeast

Individual colonies from FACS-sorted yeast plates were selected and expanded in yeast growth media to a cell density of 10⁷ cells mL⁻¹. The yeast plasmids were isolated using Easy Yeast Plasmid Isolation Kit (Clontech) per the manufacturer's instructions. The affibody sequences from the plasmids were amplified by PCR in an Applied Biosystems Thermocycler (Fisher Scientific) using HiFi PCR Premix (CloneAmp) and forward primer (5′-CCCTCAACAACTAGCAAAGG-3′; SEQ ID NO: 75) and reverse primer (3′-ATGTGTAAAGTTGGTAACGGAACG-5′; SEQ ID NO: 76) for 35 cycles and purified using a DNA Clean and Concentrator Kit (ZymoGen). The purified products were submitted for Sanger Sequencing to GeneWiz® (Azenta Life Sciences).

### Monoclonal Affibody Yeast Characterization

The binding affinity of each unique affibody for BMP-2 was characterized using flow cytometry. Samples were prepared similarly to the FACS procedure with the following differences: 1×10⁶ induced cells were used in each tube instead of 40×10⁶ cells, c-myc+bBMP-2 tubes were prepared with bBMP-2 concentrations ranging from 0.5-1000 nM, and each tube was resuspended in 200 μL of PBSA and transferred to a 96-well plate. Flow cytometry was performed on bBMP-2-containing samples in triplicate. Cells were analyzed using Accuri™ C6 Plus Flow Cytometer with 96-well plate autosampler (Becton Dickinson).

To quantify the equilibrium dissociation constant (K_{D}) of affibody-BMP-2 binding, the ratio of AF647+/AF488+ cells to AF647+ cells was calculated at each bBMP-2 concentration and plotted against protein concentration. Nonlinear regression was performed, in which the equilibrium dissociation constant was the inflection point of the curve.

Specificity of the affibodies to BMP-2 was confirmed using flow cytometry in a similar manner, except that 1 μM solutions of bVEGF (R&D Biosystems), bIL-4 (Acro Biosystems), and bGM-CSF (Acro Biosystems) were used.

Transformation of BMP-2-Specific Affibodies into E. coli

pET28b+ expression vectors containing sequences for each of the unique BMP-2-specific affibodies modified with a methionine at the N-terminus and a 6-His-tag and cysteine at the C-terminus were prepared by GenScript. The pET28b+ vector confers kanamycin resistance and uses an isopropyl ß-D-1-thiogalactopyranoside (IPTG)-inducible T7 promoter for protein expression. Vectors were transformed into BL21 chemically competent E. coli (New England BioLabs) per the manufacturer's protocols. 100 μL of transformed E. coli were plated on kanamycin selective growth plates (10 g yeast extract, 20 g bacto peptone, 20 g dextrose, 16 g bacto agar, 50 mg kanamycin sulfate, 1 L RO water) and incubated at 37° C. for 24 h. Colonies were selected and expanded in 20 mL Luria-Bertani (LB) broth (Thermo Fisher) supplemented in 20 μL of 50 mg mL⁻¹ of kanamycin sulfate in water until an optical density at 600 nm (OD600) of 0.8 was reached. 4 mL of the culture were lysed and used to obtain plasmid DNA for sequence confirmation (Plasmid Miniprep Kit; Zymo Research), and the remaining volume was split in half, in which one half was induced with 10 μL of IPTG 0.5 M and incubated further for 4 hours at 37° C., and the other half was refrigerated at 4° C. The induced and uninduced E. coli were lysed using Bug Buster Protein Extraction Agent (Millipore Sigma) and centrifuged to separate the soluble proteins and the lysate. The soluble proteins were prepared for SDS-PAGE by diluting 18 μL of sample in 6 μL Laemmli buffer (BioRad) supplemented with 10 v/v % β-mercaptoethanol (BioRad) and heated for 5 minutes at 90° C. The sampled were loaded into a 4-20% Mini-PROTEAN® TGX™ Precast Protein Gel (BioRad), run under denaturing conditions at 200 V for 35 minutes, stained with Coomassie blue dye, and imaged on an Azure 200 Gel Imager (Azure Biosystems, Inc.).

### Collection and Purification of Soluble BMP-2-Specific Affibodies

Transformed E. coli were grown in 20 mL LB broth supplemented with kanamycin to a 0.5 mM concentration and incubated overnight at 37° C. The contents were then transferred into 1.8 L of Terrific Broth (TB) supplemented with kanamycin to 0.5 mM and 500 μL of anti-foam 204 (Thermo Scientific) and cultured at 37° C. in a LEX-10 bioreactor (Epiphyte3). When the OD600 reached approximately 1.4, 1.8 mL of 0.5 M IPTG was added to the growth vessel to obtain a final concentration of 0.5 μM, and the temperature was reduced to 18° C. for 18 hours for induction of protein expression. After 18 hours, the culture was centrifuged for 20 minutes at 4° C. at 6000 RPM, and the cell pellet was removed and transferred to two 50 mL conical tubes. Binding buffer (50 mL of 1 M tris pH 7.5, 100 mL of 5 M NaCl, 5 mL of 1 M imidazole, and 845 mL RO water) supplemented with 75 mg of tris (2-carboxyethyl) phosphine hydrochloride (TCEP; GoldBio) was added to the cell pellet to a volume of 35 mL and lysed using a probe sonicator (Fisher Scientific) for 5 minutes in an ice bath. The sonicated product was centrifuged at 13,000 rcf for 30 minutes at 4° C. The supernatant was transferred to a 50 mL conical tubes along with 3.6 mL of Nickel-NTA Agarose Beads (GoldBio; Nickel beads) and rotated at 4° C. for 45 minutes. The supernatant was then transferred to a Econo-Column® chromatograph column (Biorad), washed with 50 mL of wash buffer (50 mL of 1 M tris pH 7.5, 100 mL of 5 M NaCl, 30 mL of 1 M imidazole, and 820 mL RO water) supplemented with 125 mg of TCEP followed by with 50 mL of wash buffer without TCEP, and eluted into 10 mL of elution buffer (50 mL of 1 M tris pH 7.5, 100 mL of 5 M NaCl, 250 mL of 1 M imidazole, and 600 mL RO water). The collected solution was then buffer-exchanged into 0.5 M Tris pH 8 using a 3 kDa molecular weight cut-off (MWCO) centrifuge filter (Millipore) and frozen at −80° C. until further use. SDS-PAGE was used to determine purity of the affibody at each step. UV-vis spectroscopy (Implen NP80) at 280 nm was used to determine the final concentration of the affibodies.

### Circular Dichroism of Pure and Soluble Affibodies

A Jasco J-815 circular dichroism spectropolarimeter (CD Spec; JASCO) was used to characterize the secondary protein structure of the affibodies. Purified affibody was buffer exchanged into PBS pH 6.92 using Zeba columns and diluted to a concentration below 50 μM. The protein was loaded into a quartz cuvette with 1 mm path length and placed in the CD Spec. The circular dichroism value and the high-tension voltage were collected over a wavelength range of 190-250 nm. The circular dichroism was converted to molar ellipticity using the molecular weight and concentration of each affibody.

### Native Ion Mass Spectrometry of Pure and Soluble Affibodies

A Waters Synapt G2Si mass spectrometer, calibrated with CsI cluster ions, was used to characterize the purity and mass of the collected affibodies. For each affibody, 1 mL of approximately 0.1 mg mL⁻¹ was buffer-exchanged into 200 mM ammonium acetate pH 7.52 via 6 kDa molecular weight cut-off Micro Bio-Spin 6 Columns (BioRad) and diluted to approximately 20 μM. Mass spectra were collected over 1-5 minutes using nano-electrospray ionization at a capillary voltage of 0.7-1.0 kV. Samples were deconvolved in UniDec¹¹⁵ using charge states 3 to 7 and an output mass range of 5,000-9,000 Da.

### Characterizing Binding Interactions of Pure and Soluble Affibodies Via BioLayer Interferometry

The binding interaction between BMP-2 and each soluble affibody was measured using a Gator® Plus biolayer interferometer (BLI; Gator Bio). Biotinylated BMP-2 was buffer-exchanged into PBS and diluted to 25 nM in PBS with 0.05% Tween20 (PBST; Thermo Fisher). Each soluble affibody was also buffer-exchanged into PBS and diluted to concentrations between 0-125 nM in PBST. Streptavidin-coated BLI probes (Gator Bio) were pre-soaked in 250 μL PBST for 45 minutes. The probes were then baselined with 200 μL PBST for 300 seconds and loaded with bBMP-2 for 90 seconds or until the wavelength shift plateaued. A new baseline was established using 200 μL PBST for 90 seconds, followed by 300 seconds of association with 200 μL of the various concentrations of affibody and dissociation for 300 seconds in 200 μL PBST. The association and dissociation data for the first 120 seconds were used to avoid confounding nonspecific binding interactions. One probe was loaded with bBMP-2 and no affibodies and another probe was loaded with 125 nM of affibody and no bBMP-2 for use as a reference probe and to quantify nonspecific binding to the probes, respectively.

### Computational Prediction of Binding Interaction Between BMP-2 and Pure Affibodies

The high- and low-affinity affibody sequences were input into AlphaFold2,⁶⁹ which outputs high-ranking protein structures and their corresponding prediction confidence as determined by AlphaFold2's deep learning network. The five highest ranked affibody structure prediction models for each unique affibody sequence were energetically minimized using Rosetta build 314.^{64-67,69-72} Specifically, a full-atom refinement application called Relax was used, which samples backbone and sidechain conformations to make local optimizations to the protein structure based on physics and heuristics-based weighted calculations.^{71,76,116} Furthermore, the Relax protocol constrains the minimization movements to input structure, thereby biasing the refinements to the AlphaFold2 structure predictions. The affibody binding sites and orientations to target protein BMP-2 (PDB ID: 3BMP) were then modeled using the publicly available web server for ZDOCK, a docking application which approximates global binding.⁷³ The ten most probable affibody-BMP-2 complexes determined in the ZDOCK 3.0.2 algorithm for each sequence were similarly relaxed with Rosetta. To characterize the predicted interactions from ZDOCK, we performed interface analysis using PyMOL.⁷⁴ The x-ray crystallography structure of BMP Receptor Type-1A was used as present in the RSCB protein databank (PDB: 1REW). The AlphaFold2-Multimer tool was also used to predict the docking of BMP Receptor II (BMPR-II) using PDB: 7PPA onto BMP-2 using PDB: 3BMP.^{117,118}

### Cell Culture

High glucose Dulbecco's modified eagle medium (DMEM; Gibco) was supplemented with fetal bovine serum (FBS; Bio-techne) to either 1 v/v % or 10 v/v % to create low serum or high serum medium, respectively. Both media were supplemented with 1 mL of penicillin-streptomycin solution (Millipore Sigma) containing 10,000 U mL⁻¹ penicillin and 10,000 μg mL⁻¹ streptomycin. C2C12 immortalized murine skeletal myoblasts (CRL-1772; ATCC) were maintained in high serum medium, detached and passaged using 0.25% trypsin-EDTA (Lonza), and reseeded into T75 flasks (NEST Scientific) at a density of 2,500 cells cm⁻². Cell number and viability were quantified using a Countess II Automated Cell Counter (Invitrogen).

### C2C12 Cytocompatibility Assay for Affibodies

C2C12 myoblasts were seeded onto a 96-well plate at a concentration of 2000 cells cm⁻² in 180 μL of high serum medium and allowed to adhere for 6 h. Affibodies were buffer-exchanged into PBS using 7 kDa Zeba columns, sterile-filtered through 0.22 μm filters, and diluted in sterile Dulbecco's PBS. Affibodies were added to the cell culture wells at final concentrations of 10 nM, 20 nM, 40 nM, 80 nM, or 800 nM and incubated for 72 h. The cells were washed with PBS and stained for 30 minutes at 37° C. with fresh high serum medium containing 4 mM Calcein AM (Fisher Scientific) and 2 mM ethidium homodimer-1 (Santa Cruz Biotechnology) to quantify the number of live and dead cells, respectively. Cells were imaged using a LionHeart FX automated microscope (BioTek). The number of live and dead cells were quantified using a custom script developed for Python. Cell viability was calculated by dividing the number of living cells by the total number of cells.

### C2C12 Alkaline Phosphatase Activity Assay

C2C12 myoblasts were seeded onto a 96-well plate at a concentration of 62,500 cells cm⁻² in 200 μL of high serum medium and allowed to adhere for 6 h, after which the cells were washed with PBS and resuspended in 100 μL of low serum media containing the different treatments. Affibodies were buffer-exchanged into PBS using 7 kDa Zeba columns, sterile-filtered through 0.22 μm filters, and diluted in PBS to concentrations of 10 nM, 20 nM, 40 nM, 80 nM, and 1000 nM. Sterile carrier-free recombinant human BMP-2 (R&D Biosystems) was diluted to 20 nM in PBS. 20 nM of BMP-2 and/or 10 nM, 20 nM, 40 nM, 80 nM, or 1000 nM of affibodies were added sequentially for the “uncomplexed” treatment groups or as a premixed solution for the “complexed” treatment groups as described above. After 72 h, the cells were lysed with Cellytic M (Millipore Sigma), and their ALP activity was quantified and normalized to the total amount of double stranded DNA (dsDNA) present in each well.⁸⁸ For the ALP colorimetric assay, 50 μL buffer solution consisting of equal volumes of 1.5 M 2-amino-2methyl-1-propanol solution pH 10.25, 20 mM p-nitrophenyl phosphate solution, and 10 mM MgCl₂ hexahydrate solution were mixed with 50 μL of lysed cells and incubated in the dark for 20 minutes before the absorbance of the solutions was read at 405 nm (Synergy Neo2, Biotek). The colorimetric change in the solutions was converted to p-nitrophenol concentration using a calibration curve of 0-0.8 μmol mL⁻¹ 4-nitrophenol solution (Millipore Sigma). The QuantiFluor dsDNA System (Promega) was used for dsDNA quantification. The ALP activity in each well was normalized to the dsDNA content to account for variability in cell number between samples.

### Synthesis of Affibody-Conjugated Poly(Ethylene Glycol)-Maleimide Hydrogels

Affibodies were buffer-exchanged into PBS pH 6.92 using 7 kDa Zeba columns. A 16.7 w/v % solution of 20 kDa 4-arm PEG-Mal (Laysan Bio) in PBS pH 6.92 was prepared. 30 μL of PEG-Mal solution was mixed with 1.92 nmol of affibody in 30 μL PBS pH 6.92 or with 30 μL of PBS pH 6.92 for the negative control. The solution was rotated for 1 hour at room temperature for affibody conjugation. 40 μL of DTT (GoldBio) solution (1.93 mg mL⁻¹ in PBS pH 6.92) was added to each tube containing PEG-Mal and rotated at room temperature for 30 minutes to form 100 μL 5 w/v % PEG-Mal hydrogels with/without 1.92 nmol of affibodies.^{56,92} The hydrogels were washed three times with 500 μL of PBS for 6 h to remove unreacted DTT and affibody. To maintain sterility for sterile hydrogels, the PEG-Mal, DTT, and PBS solutions were sterile-filtered with a 0.22 μm syringe filter and handled in a biosafety cabinet prior to mixing and crosslinking.

Encapsulation and Controlled Release of BMP-2 from Affibody-Conjugated PEG-Mal Hydrogels

100 μL 5 w/v % PEG-Mal hydrogels were prepared without affibody and with each of the two unique BMP-2 affibodies as described above. After purification, the PBS supernatant was removed and 20 μL of 5 μg mL⁻¹ BMP-2 in PBSA were pipetted onto each hydrogel. The tubes were rotated at 4° C. for 12 h to allow BMP-2 to infiltrate the hydrogels. The hydrogels were washed with PBSA twice. 880 μL of PBSA was added to each hydrogel for a total tube volume of 1 mL. The hydrogels were placed at 37° C. and timepoints were collected by removing 200 μL of supernatant from the tube and replenishing it with fresh PBSA. BMP-2 in the washes and collected timepoints was quantified using a Human BMP-2 DuoSet ELISA kit (R&D Biosystems). For BMP-2 release into serum, 10 v/v % of FBS in PBS was used as the solution for time 0 and onward.

Encapsulation efficiency was calculated by comparing the total amount of BMP-2 collected from each hydrogel in wash 1 and wash 2 with the total amount of BMP-2 added to the hydrogel. Cumulative release at each timepoint was calculated by dividing the total amount of BMP-2 collected from each hydrogel by the amount of BMP-2 encapsulated in each hydrogel. The effective diffusivity (i.e., release rate) of the BMP-2 from the hydrogels was calculated using a Fickian diffusion model from a thin polymeric sheet in a pseudo-infinite surrounding volume.¹⁰⁵

BMP-2 Bioactivity Upon Release from Affibody-Conjugated PEG-Mal Hydrogels

200 μL sterile PEG-Mal hydrogels without affibody (PEG-Mal control hydrogel) or with each affibody (affibody-conjugated PEG-Mal hydrogels) were prepared in a biosafety cabinet as described above by doubling the quantities of all reagents in each hydrogel. 10 μg mL⁻¹ of sterile BMP-2 in low serum media was added to each hydrogel and rotated overnight at 4° C. to allow the BMP-2 to infiltrate the hydrogels. 800 μL of low serum media was added to each hydrogel formulation and incubated at 37° C. 230 μL of the supernatant were collected and replenished with fresh low serum media at 1, 2, 3, 5 and 7 days.

Meanwhile, C2C12 myoblasts were cultured as described above. After all the timepoints were collected, the cells were washed and detached with 0.25% trypsin-EDTA, and the wells of a 96-well plate were seeded with 62,500 cells cm⁻² in 200 μL of high serum medium for 6 h to adhere. The medium was then removed, the cells were washed with PBS, and 200 μL of each collected timepoint was added to the cells for 72 hours. ALP activity was quantified as described above. The remaining 30 μL of each collected sample were used to quantify the amount of BMP-2 added to each well using BMP-2 ELISA. ALP activity was normalized to dsDNA and the amount of released BMP-2 added to each well. Area under the curve was calculated for each group as the sum of normalized activity for the duration of the experiment.

### Statistical Analysis

Data pre-processing was performed using GraphPad Prism 9.5.1, except flow cytometry data which was prepared using FlowJo 10.8.1, biolayer interferometry preparation and curve fitting which was prepared using GatorOne 2.10, and protein structural presentation which was prepared using PyMOL 4.6.0. All relevant data are reported as means+/−standard deviation with sample sizes indicated in the figure caption. All statistical methods used to assess significant differences and applicable post-hoc tests are reported in figure descriptions. For all data, the use of one-way ANOVA was chosen for a single variable of comparison. For all two-way ANOVA, multiple variables were compared. Tukey post-hoc tests were performed to compare the significance of all groups between each other. Dunnett post-hoc tests were performed to compare the significance of all data to a control group.

### Example 2: Magnetic-Activated Cell Sorting Depleted Over 99% of the Yeast Display Library Diversity

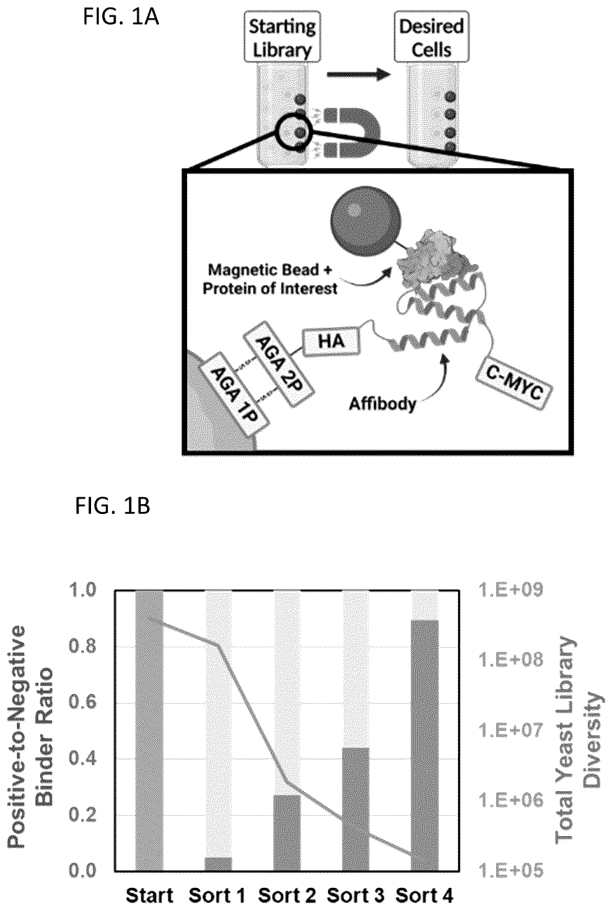

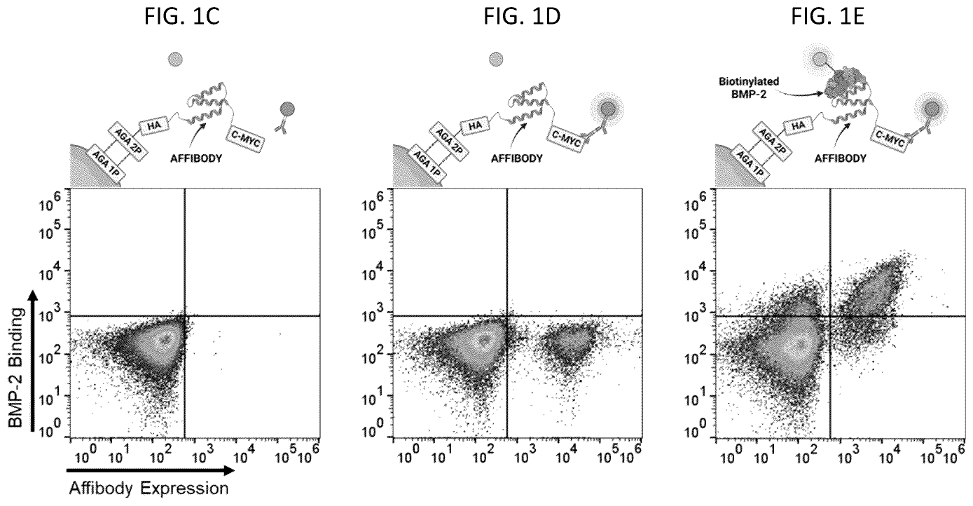

Four rounds of magnetic-activated cell sorting (MACS) were performed to enrich for BMP-2-binding affibodies within the yeast surface display library (FIG. 1A). Each round of MACS consisted of two negative magnetic bead sorts to remove non-specific protein binders^{39,40,50,51} and one positive magnetic bead sort using beads conjugated with BMP-2 to enrich for yeast displaying BMP-2-specific affibodies. Following each round of MACS, yeast from each bead sort were plated on selective growth plates to count the number of colonies that bound to the negative beads and BMP-2-conjugated beads.

The new yeast library diversity after each round of MACS was estimated by counting the number of colonies grown on the BMP-2 plates, while the ratio of positive-to-negative binders was calculated by dividing the number of colonies grown on the BMP-2 plates by the sum of colonies grown on the negative plates. FIG. 1B demonstrates how each round of MACS enriched BMP-2 specific affibodies (blue bar graph) while reducing the total yeast library diversity (i.e., number of unique variants) (orange line plot). After four rounds of MACS, the library diversity was reduced by over 99%, and the number of BMP-2-specific affibodies was estimated to account for approximately 89% of the remaining library.

### Example 3: Fluorescence-Activated Cell Sorting Identified BMP-2-Specific Affibodies

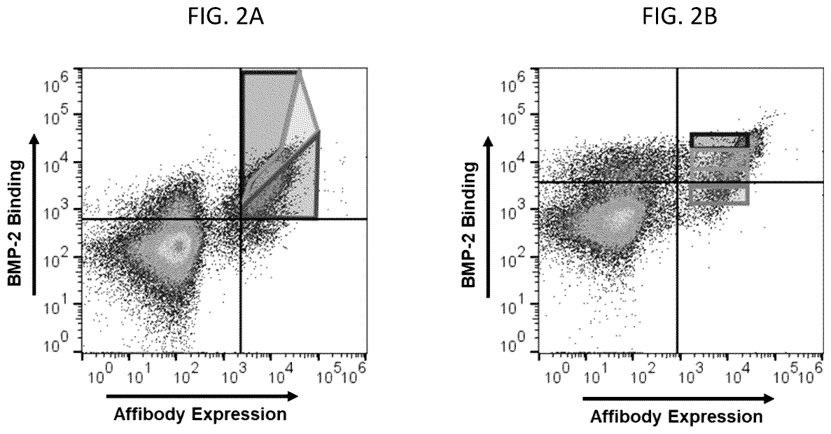

Following four rounds of MACS, fluorescence-activated cell sorting (FACS) was performed on the enriched yeast library, which was gated into populations corresponding to different affinity ranges for BMP-2 binding. Yeast were incubated in 0.1 mg mL⁻¹ bovine serum albumin (BSA) in phosphate buffered saline (PBS) (i.e., PBSA) without fluorescent tags or proteins (cells-only control) (FIG. 1C), with a mouse anti-c-myc antibody (αCMYC, 9E10) to assess affibody expression levels by binding to the N-terminal c-myc epitope (FIG. 1D), or with both αCMYC and biotinylated BMP-2 (bBMP) to assess BMP-2 binding to displayed affibodies (FIG. 1E). Except for the cells-only control, all yeast were incubated with secondary fluorescent tags that bound specifically to αCMYC (AlexaFluor 647 goat anti-mouse conjugate; AF647) or bBMP (AlexaFluor 488 streptavidin conjugate; AF488). AF647+/AF488+ yeast cells were gated using two gating approaches and collected (FIGS. 2A-2B). At least 10,000 yeast cells were collected from each gate during FACS to capture all unique affibody sequences from each gate.

Following FACS, yeast from each gate were plated onto selective growth plates and allowed to form discernable colonies that each contained a single affibody sequence (i.e., monoclonal yeast). Three colonies from each gate were grown in growth media for a total of 21 yeast clones. Sanger sequencing of plasmid DNA revealed 11 unique affibody sequences (SEQ ID NOS: 1 to 11).

### Example 4: Characterization of BMP-2 Binding to Monoclonal Yeast Affibodies

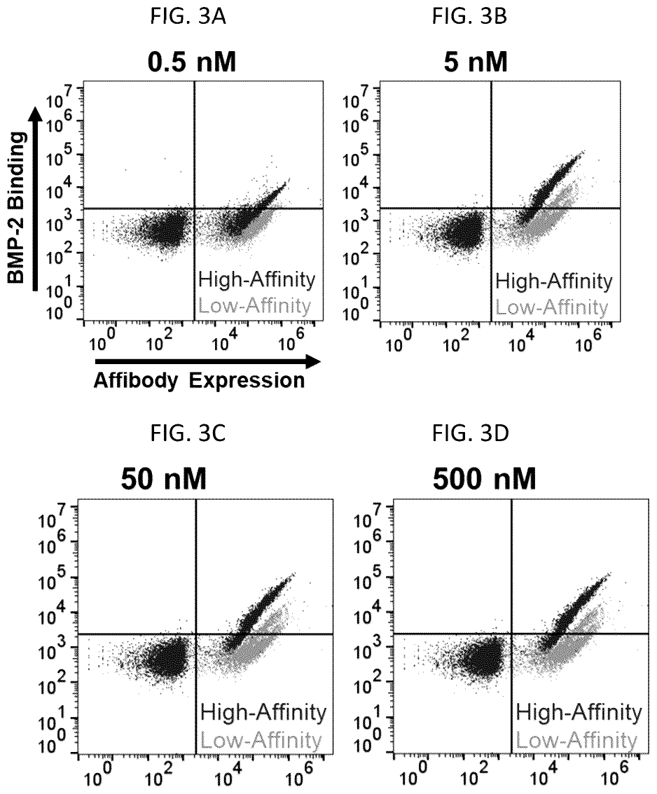

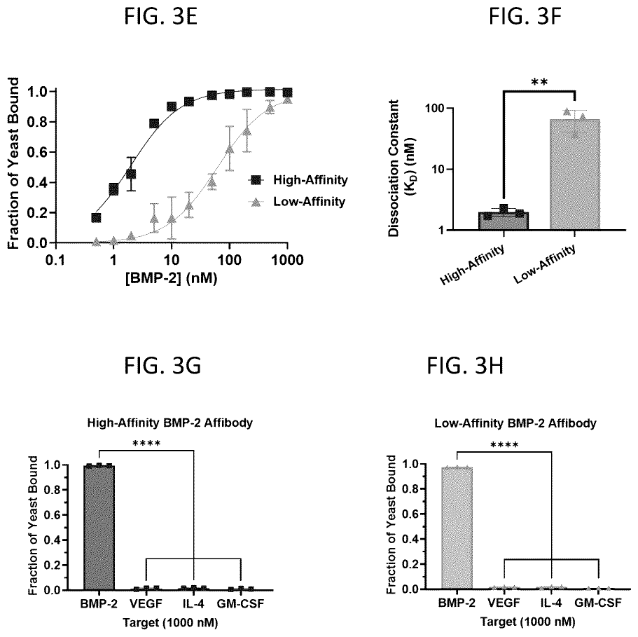

Binding affinities between BMP-2 and BMP-2-specific affibodies were assessed on yeast using flow cytometry. Similar to FACS, monoclonal affibody-displaying yeast were incubated in either PBSA, αCMYC and secondary solution, or αCMYC with a range of bBMP concentrations (0.5-1000 nM) and secondary solution (AF647 and AF488 for affibody expression and bBMP-2 binding, respectively). At each concentration of bBMP, the fraction of displayed affibodies that were bound to BMP-2 was determined by dividing the top right quadrant (AF647+/AF488+) by the right half of the graph (AF647+). With increasing bBMP-2 concentrations, more cells were labeled with AF488, resulting in an upward shift of the population that indicated increased bBMP-2 binding (FIGS. 3A-3D). Binding affinity was assessed by plotting the fraction of bBMP-2 bound over the bBMP-2 concentration range (FIG. 3E). Monoclonal yeast that demonstrated a greater bBMP-2 binding had higher affinities for BMP-2.⁵² Equilibrium dissociation constants (K_{D}) were calculated by performing a nonlinear regression on bBMP-2 binding to affibody-displaying yeast at varying bBMP-2 concentrations (FIGS. 3E-3F).

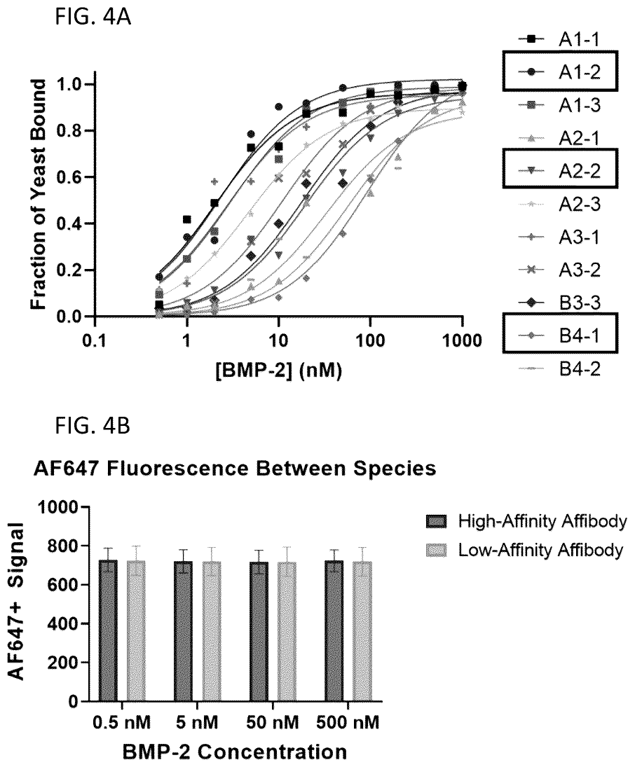

The affinities of all 11 unique clones that bound to BMP-2 (SEQ ID NOS: 1-11) were quantified (FIG. 4A) and two unique clones (SEQ ID NOS: 1 and 3) were chosen for further examination. These clones displayed significantly different affinities for BMP-2 and will be identified hereafter as high-affinity (K_{D}=1.95±0.14 nM) and low-affinity (K_{D}=61.82±9.38 nM) BMP-2-binding affibodies. The affinities between the other affibodies and BMP-2 were found to be within the range of the equilibrium dissociation constants of the high- and low-affinity affibodies. The quantity of surface-displayed affibodies may have affected the perceived affinity between the affibodies and BMP-2 by altering the ratio between the proteins and the protein-binding partners.⁵² The average AF647+ fluorescence signal, indicative of affibody expression, was similar between the high- and low-affinity affibodies at each BMP-2 concentration, confirming a comparable number of affibodies displayed on the surface of each monoclonal yeast species (FIG. 4B).

Specificity of the high- and low-affinity affibodies for BMP-2 was also assessed using flow cytometry. Several other proteins involved in the bone healing cascade were chosen to investigate specificity of the BMP-2 affibodies. Monoclonal affibody-displaying yeast were incubated with PBSA, αCMYC and secondary solution, or αCMYC with 1000 nM of biotinylated vascular endothelial growth factor (bVEGF), biotinylated interleukin-4 (bIL-4), or biotinylated granulocyte-macrophage colony stimulated factor (bGM-CSF) and secondary solution. All affibodies exhibited negligible binding to bVEGF, bIL-4, and bGM-CSF, demonstrating that these affibodies were specific to BMP-2 (FIGS. 3G-3H).

### Example 5: Collection and Characterization BMP-2-Specific Affibodies

Sequences for the high- and low-affinity affibodies (SEQ ID NOS: 1 and 3) modified with a 6-histidine (His-tag) for protein collection^{39,53,54} and a N-terminal cysteine for bioconjugation⁵⁵⁻⁵⁷ were ligated into a pET28b+ expression vector, which was transformed into chemically competent BL21 E. coli for protein expression. Soluble protein was collected using benchtop immobilized metal affinity chromatography (IMAC) with cobalt-nitrilotriacetic acid beads.⁵³ Approximately 10 mg of pure soluble affibodies were collected from each liter of E. coli culture.

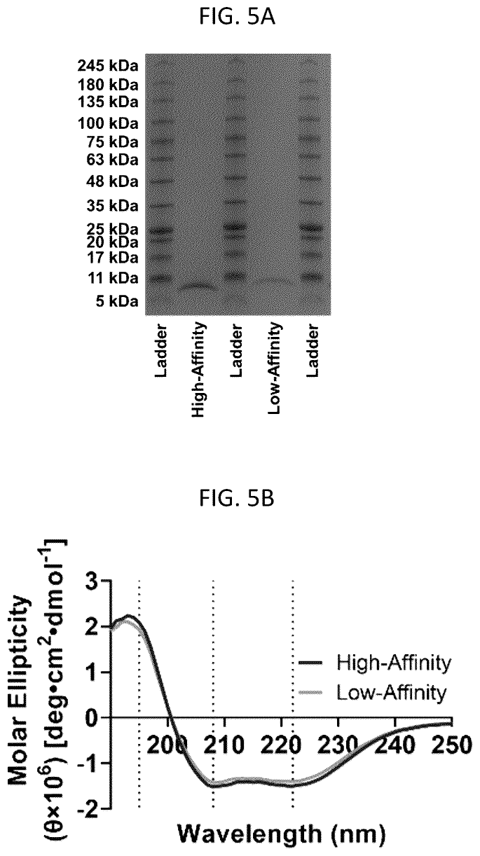

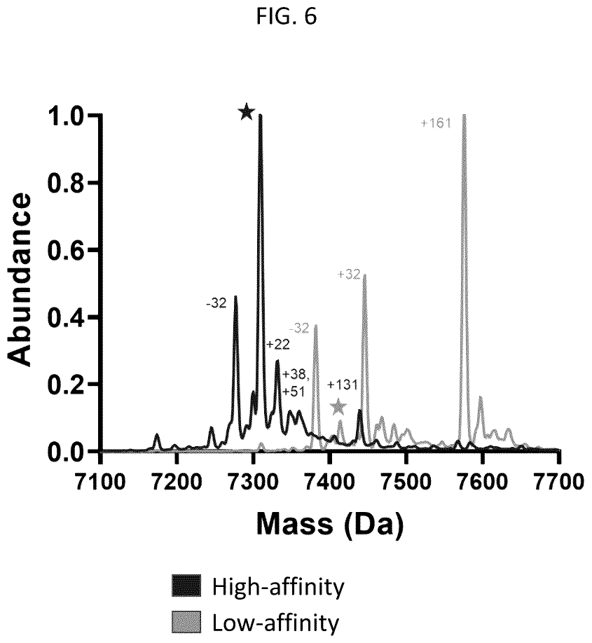

Sodium dodecyl sulfate polyacrylamide gel electrophoresis (SDS-PAGE) (FIG. 5A) and native ion mass spectrometry (NIMS) (FIG. 6) were used to determine the size of the affibodies. SDS-PAGE of purified affibodies (150 μM in tris, pH 8) revealed thick bands visible between the 5 kDa and 11 kDa rungs of the control ladder at the approximate expected sizes of the two affibodies (7308 Da and 7414 Da for the high- and low-affinity affibodies, respectively) without any other noticeable bands. Native ion mass spectrometry (NIMS) of affibodies (20 μM in 0.2 M ammonium acetate, pH 7.52) demonstrated a dominant high-affinity affibody peak corresponding to the expected mass of 7308 Da with other well-populated peaks associated with sodium and potassium adducts, the possible formation of a cysteic acid or piperidine on the C-terminal,⁵⁸ and glutamylation of the N-terminal cysteine. The low-affinity affibody (SEQ ID NO: 3) displayed a small peak at the expected mass of 7414 Da and had prominent peak shifts associated with the formation of a dehydroalanine,⁵⁹ a 32 Da shift attributed to a trisulfide bond,⁶⁰ an additional shift associated with a piperidine formation on the terminal cysteine,⁵⁸ and another prominent peak shift (161 Da) attributed to a carboxymethyl cysteine or carboxymethyl cystenyl.⁶¹ These data indicate that these affibodies may readily undergo post translational modifications and that the low affinity affibody may undergo extensive post-translational modification. However, these modifications mainly affect the terminal cysteine, which may affect chemical conjugation of the affibody to biomaterials, but are not expected to affect affibody binding affinity for BMP-2.⁵⁸

Circular dichroism was used to determine the secondary structure of the affibodies.⁶² Affibodies were diluted to concentrations between 17-30 μM in 5 mM tris pH 6.92, which was a pH equidistant from each of their isoelectric points. Both affibodies exhibited characteristic α-helical profiles, including troughs at 208 nm and 222 nm and a peak at 195 nm,^{40,62} confirming the secondary structure of the affibodies in their soluble state (FIG. 5B).⁶³

### Example 6: Characterization of Soluble Affibody-BMP-2 Binding Interactions

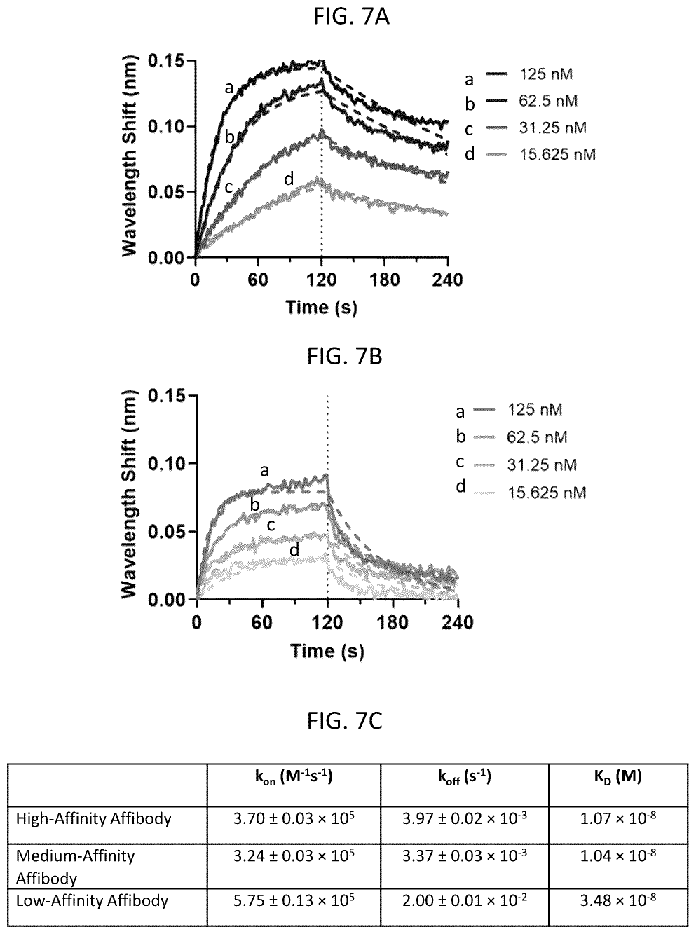

The binding interaction between the soluble high—(SEQ ID NO: 1) and low-(SEQ ID NO: 3) affinity affibodies and BMP-2 were characterized using biolayer interferometry (BLI). Streptavidin-coated BLI probes were coated with 25 nM bBMP-2 in PBS with 0.05% Tween-20® (PBST), followed by association of 0-125 nM of the purified soluble affibody in PBST for 120 seconds and dissociation in PBST for 120 seconds (FIGS. 7A-7B). BLI enabled the determination of the dissociation rate constant (k_{off}), association rate constant (kₒₙ), and overall equilibrium dissociation constant (K_{D}) of each binding interaction. The equilibrium dissociation constants of the high-, medium-, and low-affinity affibodies (SEQ ID NO: 1, 2 and 3, respectively) for BMP-2 were determined to be 10.7 nM, 10.4 nM, and 34.8 nM, respectively (FIG. 7C). The k_{off} of the low-affinity affibody was an order of magnitude higher than that of the high-affinity affibody, and the low-affinity affibody completely dissociated from the bBMP-2. While evaluating BMP-2-affibody binding on the surface of the yeast provided insight to the binding strength of the affibody, it was more representative of the avidity (i.e., total binding strength) rather than affinity of the individual affibodies and did not provide information about the association and dissociation rates of the binding interaction.⁵² As such, BLI provided a more representative measurement of affibody affinity for BMP-2 when integrated into hydrogels.

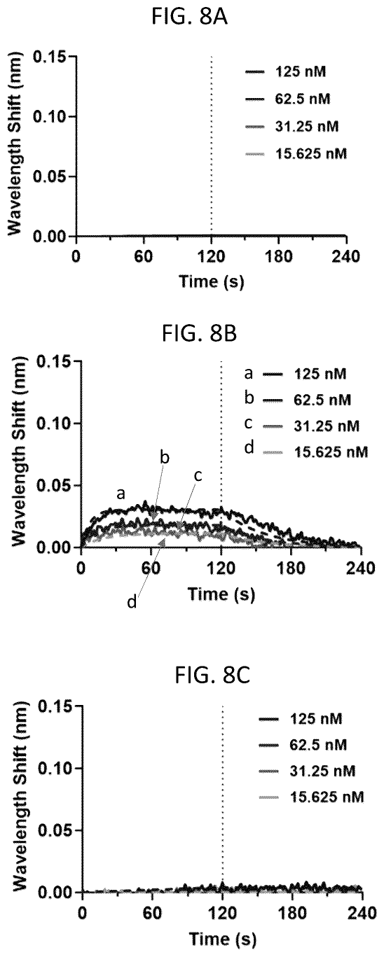

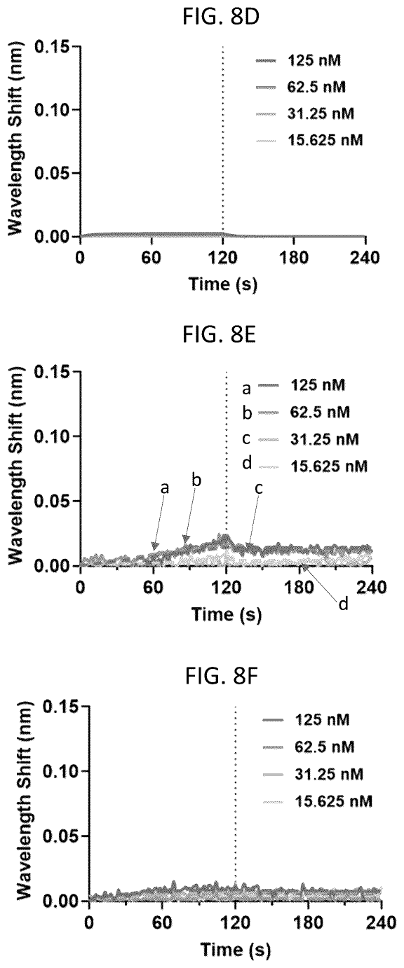

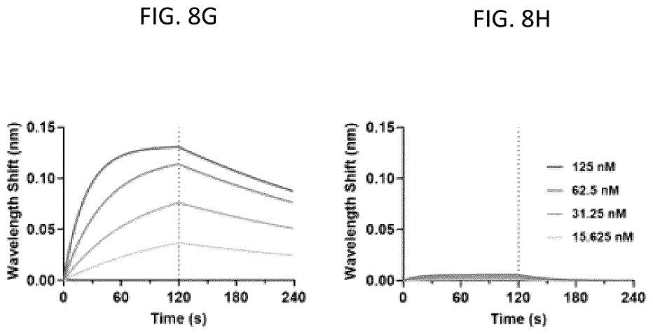

BLI was also performed using streptavidin-coated probes coated with 25 nM of bVEGF, bIL-4, or bGM-CSF followed by association and dissociation of 0-125 nM of high-, medium- and low-affinity affibody in PBST (FIGS. 8A-8H). There was no noticeable binding response to VEGF and GM-CSF, and only a minimal binding response to IL-4, indicating that the affibodies do not bind to VEGF or GM-CSF and bind minimally to IL-4 when compared to BMP-2. Overall, these results demonstrate that the soluble affibodies specifically bind to BMP-2 and that the high-affinity affibody (SEQ ID NO: 1) has a stronger interaction with BMP-2 compared to the low-affinity affibody (SEQ ID NO: 3).

### Example 7: Computational Predictions of Affibody Binding to BMP-2

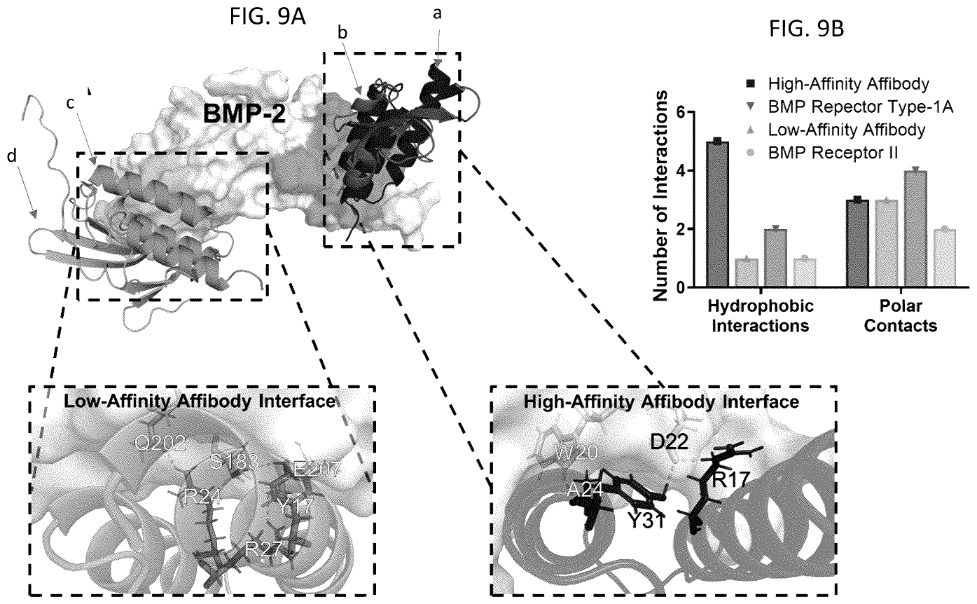

The computational tools AlphaFold 2, ZDOCK, and Rosetta were used to predict the site of interaction between each affibody and BMP-2. AlphaFold predicted the folded structures of the affibodies with high confidence (predicted local distance difference test score >95 for most of the predictions).⁶⁴⁻⁶⁹ Predicted affibody structures were energetically minimized using a protocol in Rosetta.⁷⁰⁻⁷² These structures were then docked to BMP-2 using the ZDOCK algorithm.⁷³ The top-ranked conformations for each affibody-BMP-2 complex were visualized in Pymol (FIG. 9A).⁷⁴ The high-affinity affibody was predicted to interact with BMP-2 at the binding site commonly referred to as the “wrist,” while the low-affinity affibody was predicted to bind to a different site of BMP-2 known as the “knuckle.”^{1,75} Electrostatic interactions were defined as polar contacts between BMP-2 and each respective affibody. Hydrophobic interactions were defined as hydrophobic amino acids of BMP-2 less than 3.5 Å away from each affibody, which structurally contributed to the formation of a hydrophobic pocket. Interfacial calculations suggested that the interactions between the high-affinity affibody and BMP-2 were governed by multiple hydrophobic and electrostatic intermolecular interactions with BMP-2 at the wrist binding site, whereas the low-affinity affibody interacted with the knuckle binding site of BMP-2 primarily through electrostatic interactions (FIG. 9B).^{76,77} In comparison to collagen which has been shown to have a >500 nM equilibrium dissociation constant with BMP-2,⁷⁸ the affibody-based electrostatic interactions with BMP-2 are an order of magnitude stronger. Furthermore, collagen interacts with many different biomolecules, making its interaction with BMP-2 less specific than the BMP-2-affibody interactions and potentially less controllable in complex in vivo environments.^{78,79}

The BMP-2 wrist binding epitope has been recognized as the binding site for BMP receptor type-1A (BMPR1A), with which the growth factor makes a relatively strong binding interaction (K_{D}≈0.7 nM),⁸⁰ while the knuckle is a relatively weak binding site for BMP receptor type II (BMPR-II) (K_{D}≈100 nM).^{1,81} BMP-2-induced osteogenesis occurs in skeletal myoblasts and mesenchymal stromal cells when a BMP-2 dimer interacts with a cell membrane-bound hetero-tetramer formed from two BMPR1A and two BMPR-II.^{1,75,82,83} These data indicate that binding of the affibody to BMP-2 at higher affinities than its receptors could potentially interfere with BMP-2-receptor binding, subsequently inhibiting BMP-2-induced osteogenesis. Additionally, increasing the quantity of affibody present in solution may shift the dynamics of receptor binding, resulting in a concentration-dependent inhibition. To test these hypotheses in vitro, C2C12 immortalized murine skeletal myoblast cell line were used. The murine cell line has been shown to express markers of early osteogenic differentiation, such as alkaline phosphate (ALP) activity, in the presence of BMP-2 in a dose-dependent manner.^{81,84,85}

### Example 8: Affibodies do not Impact C2C12 Cell Viability or Proliferation

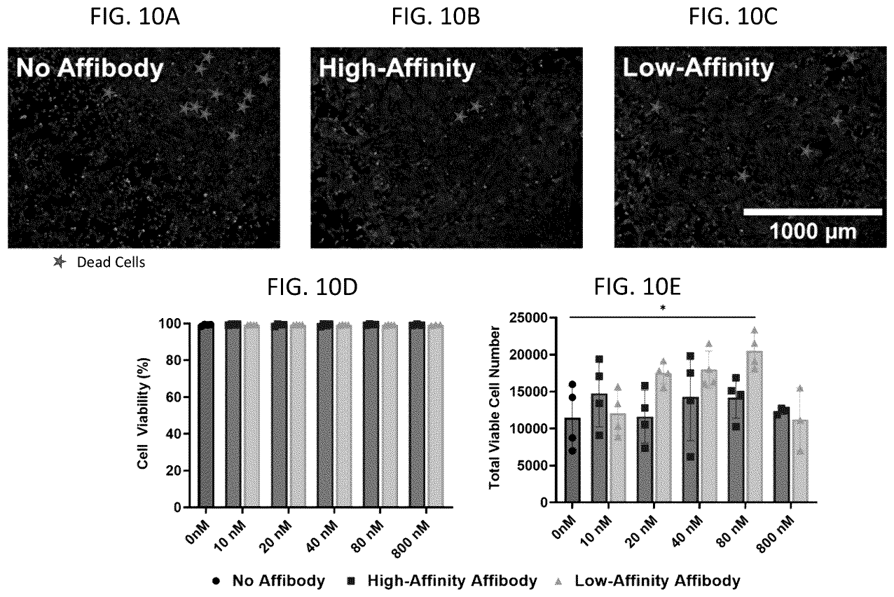

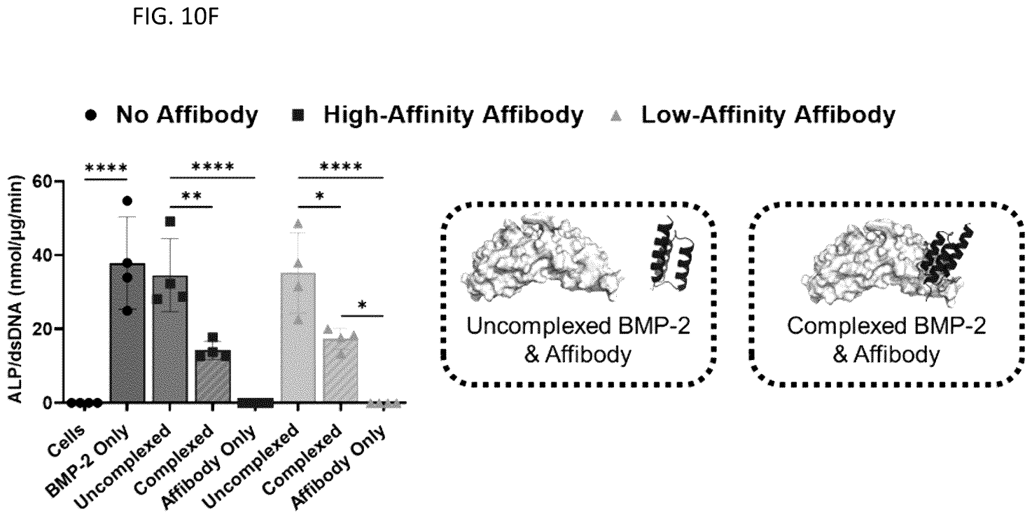

The cytocompatibility of the soluble BMP-2-specific affibodies was assessed using C2C12 cells. Soluble high- and low-affinity affibodies (SEQ ID NO: 1 and 3, respectively) were added to C2C12 cultures at final concentrations of 10 nM, 20 nM, 40 nM, 80 nM, or 800 nM. After incubation for 72 hours, cells were stained with calcein AM and ethidium homodimer-1 to quantify live and dead cells, respectively, and imaged (FIGS. 10A-10C). Cell viability was calculated by dividing the number of living cells by the total number of cells (FIG. 10D), and total cell count was calculated by averaging the number of live cells in each image (FIG. 10E). C2C12 viability was not negatively impacted by the introduction of any concentration of affibodies. The total viable cell count was also largely unaffected by the various concentrations of the affibodies. Although treatment with 80 nM of low affinity affibody increased cell number, the lack of discernable pattern indicated that cell proliferation was not affected by the affibodies.

### Example 9: Affibody-BMP-2 Binding Reduces Alkaline Phosphatase Activity of C2C12 Cells

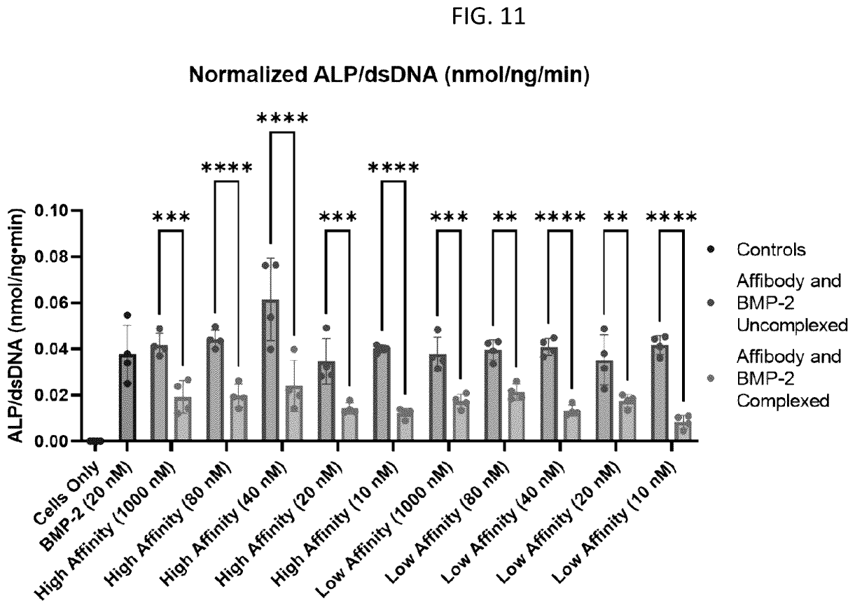

ALP activity, which is an indicator of early osteogenic differentiation,^{86,87} was used to assess the impact of affibodies on BMP-2 bioactivity. 20 nM of BMP-2 with or without different concentrations of soluble high- or low-affinity affibodies were added to C2C12 cultures sequentially for the “uncomplexed” treatment groups (affibodies first, incubated for 45 minutes, followed by BMP-2) or as a premixed solution (45 minutes of mixing to ensure adequate time for interaction) for the “complexed” treatment groups. After 72 hours, cells were lysed and their ALP activity was quantified by a colorimetric change caused by the ALP-induced catalysis of p-nitro phenyl phosphate to p-nitrophenol.⁸⁸ ALP activity was normalized to the total amount of double-stranded DNA present in each cell culture. Treatment with both uncomplexed and complexed BMP-2 and affibodies was performed to compare the different states in which BMP-2 may be presented in clinical applications. Traditionally, BMP-2 is soaked into a collagen sponge prior to delivery to a bone defect,⁸⁹ resulting in some burst release of protein and some long-term retention of BMP-2 within the scaffold. In the absence of a hydrogel or other delivery vehicle, the uncomplexed treatment group represented the released BMP-2, which would interact with cells outside of the scaffold, while the complexed treatment group represented the BMP-2 that would remain bound to the affibodies within the delivery vehicle and would interact with cells that migrate into the scaffold.^{31,90}° FIG. 10F depicts the normalized ALP activity for the experimental groups using 20 nM affibody concentrations (1:1 ratio of BMP-2 to affibody). FIG. 11 depicts additional affibody concentrations (10-1000 nM), which resulted in ALP activity that followed similar trends. No significant differences were observed between ALP activity induced by high- and low-affinity affibody-BMP-2 treatment groups. In the absence of BMP-2, no ALP activity was observed, regardless of affibody presence. In the presence of BMP-2 alone, normalized ALP activity was 37.9±12.5 nmol pNPP μg⁻¹ dsDNA min⁻¹, indicative of early osteogenic differentiation of the C2C12 cells.⁸⁴ For the uncomplexed approach, the affibodies caused an insignificant reduction in ALP activity. For all complexed treatment groups, ALP activity was significantly reduced compared to BMP-2 treatment and the uncomplexed treatment groups. These results indicate that affibody binding to BMP-2 may inhibit some of the function of BMP-2 or inhibited its interaction with the requisite tetramer complex of membrane-bound BMP receptors,^{1,75} supporting the computational docking simulations. The BLI data (FIGS. 8A-8B) indicates that some portion of the BMP-2 remained bound to the high-affinity affibody for an extended period, potentially exceeding the 72 hours incubation period. Even though the BLI data indicated that the low-affinity binder allows for complete dissociation of the BMP-2, the low-affinity binder has a greater affinity for the knuckle than BMPR-II. Since optimal BMP-2 activity requires a tetramer of two BMPR-1A and two BMPR-II,⁸¹ disruptions to the binding interactions between BMP-2 and either BMP receptor on the C2C12 cells may have restricted the induction of ALP activity in C2C12 cells.

### Example 10: Synthesis of Affibody-Conjugated Poly(Ethylene Glycol)-Maleimide Hydrogels

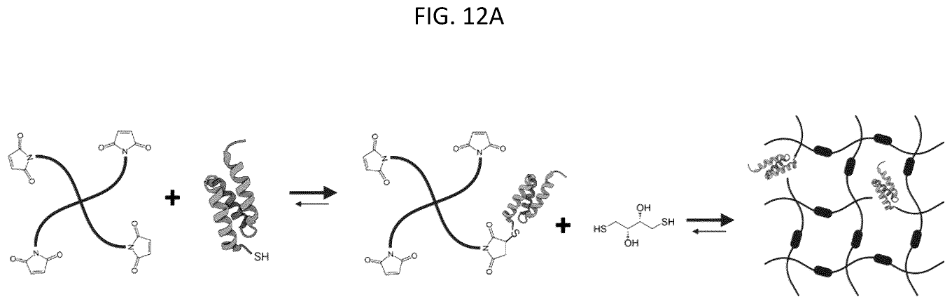

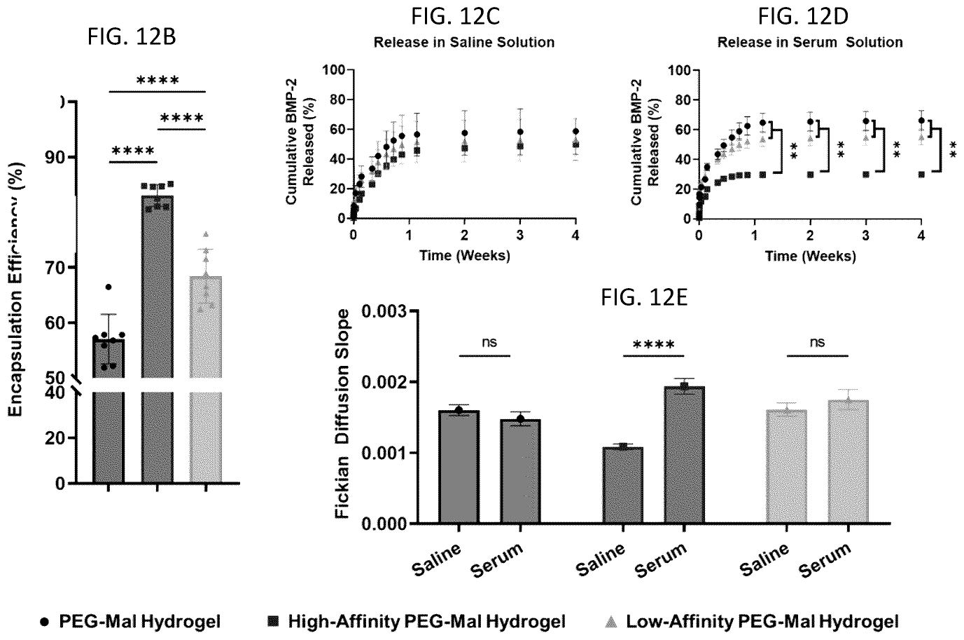

Affibody-conjugated PEG-Mal hydrogels were fabricated to assess the effect of affibody affinity on BMP-2 release from a hydrogel delivery vehicle that could be implanted similarly to the industry-used absorbable collagen sponge. 100 μL 5 w/v % PEG-Mal hydrogels containing 1.92 nmol of either high- or low-affinity BMP-2-specific affibodies were synthesized by mixing PEG-Mal with soluble affibody, where the C-terminal cysteines of the affibodies spontaneously reacted with available maleimides through a thiol-maleimide addition to form an affibody-conjugated PEG-Mal intermediate.⁹¹ The remaining maleimides were then crosslinked using dithiothreitol (DTT) to form hydrogels (FIG. 12A).⁵⁶

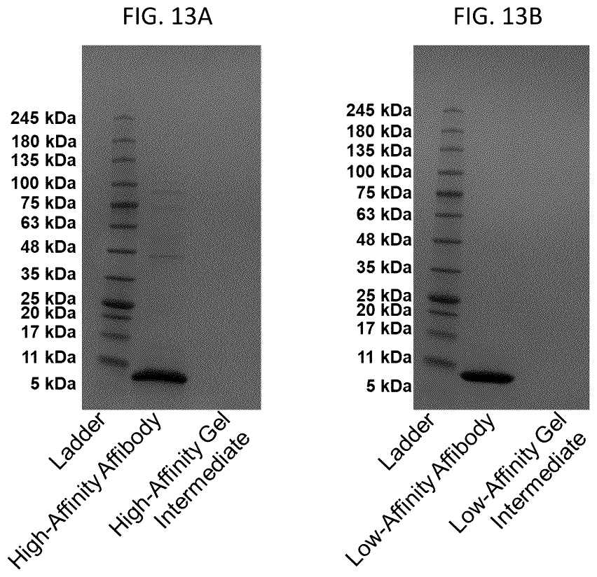

To confirm the conjugation of the affibodies to the maleimide groups, the intermediate solutions of affibody-PEG conjugates were passed through a 10 kDa molecular weight cut-off filter to separate unconjugated affibodies, and the flowthrough was subjected to SDS-PAGE. The unconjugated affibody solutions that were used underwent the same filtration and SDS-PAGE to compare the inputs and outputs of the reaction (FIGS. 13A-13B). The presence of affibodies in the pre-conjugation lanes indicated the affibodies readily pass through the filter pores. However, upon conjugation to a 20 kDa 4-arm PEG-Mal, the affibodies were no longer able to pass through the filter. The absence of a band indicated that most of the affibodies reacted with the maleimide groups and an undetectable amount of free affibodies was retained in the PEG-Mal+affibody intermediate conjugate.⁹²

### Example 11: Encapsulation and Controlled Release of BMP-2 from Affibody-Conjugated PEG-Mal Hydrogels

To assess the impact of affibodies on BMP-2 encapsulation, 100 μL 5 w/v % PEG-Mal hydrogels containing no affibodies, low-affinity affibodies (SEQ ID NO: 3), or high-affinity affibodies (SEQ ID NO: 1) were loaded with 100 ng of BMP-2 (3.85 pmol, 500×molar equivalents of affibody to BMP-2) in PBSA overnight. The next day, the hydrogels were washed in PBSA to remove unencapsulated BMP-2 and minimize variability in BMP-2 uptake. Although the timeframes are different, this method of absorbing BMP-2 into prefabricated PEG-Mal hydrogels encapsulation is similar to the clinical method procedure of absorbing BMP-2 into collagen sponges before implantation in the patient.⁸⁹ BMP-2 content in the washes and the original BMP-2 solution were quantified using enzyme-linked immunosorbent assay (ELISA). The affibody-conjugated hydrogels demonstrated a significantly higher BMP-2 encapsulation efficiency than hydrogels without affibodies (FIG. 12B). The high-affinity hydrogel demonstrated an encapsulation efficiency of 83.0±1.98%, which was higher than that of the low affinity hydrogel, which was 68.4±4.90%.

After removing the unbound BMP-2 from the hydrogels, the hydrogels were suspended in either PBSA or PBS containing 10% fetal bovine serum (FBS) to study the effect of the surrounding environment on BMP-2 release. The saline solution was used to reduce the number of variables that could affect the release, while the serum solution, which contains a variety of lipids proteins, enzymes, and other constituents, was used to more accurately mimic the in vivo environment.⁹³⁻⁹⁵ Aliquots of the supernatant were collected over four weeks, analyzed by BMP-2 ELISA, and graphed as cumulative release as a percentage of BMP-2 encapsulated (FIGS. 12C-12D). At the conclusion of the experiment, all PEG hydrogels were still intact and did not exhibit notable degradation, which is comparable similar to other PEG-based hydrogels crosslinked via Michael-type addition.⁹⁶ No significant differences were observed between BMP-2 released into saline from any of the hydrogel groups. In contrast, the high-affinity affibody hydrogels demonstrated significantly slower BMP-2 release into serum than the no affibody and low-affinity affibody hydrogels at all timepoints after 15 minutes.

The total amount of BMP-2 released from the high-affinity hydrogels was lower than that of the no affibody and low-affinity affibody hydrogels. These results corroborated the affibody-BMP-2 binding interaction data from BLI that demonstrated complete dissociation of BMP-2 from the low-affinity affibody, but incomplete dissociation of the BMP-2 from the high-affinity affibody. All hydrogel groups exhibited a plateau in BMP-2 release after approximately one week that was lower than the total amount of BMP-2 loaded into the hydrogel, which is a common observation for protein release vehicles.^{30,97,98} This could be attributed to the establishment of a protein concentration equilibrium between the hydrogel and its surrounding environment, as well as protein aggregation and conformational changes that reduced protein detection by ELISA.⁹⁹⁻¹⁰² BMP-2 has been shown to aggregate at physiological pH, even with the addition of stabilizing agents such as BSA and salts.^{103,104}

The effective diffusivity (i.e., release rate) of the BMP-2 from the hydrogels was calculated using a Fickian diffusion model,¹⁰⁵ from the slope of the linear portion of a curve comparing Mₜ/M_{∞} and root time (s^{1/2}), where Mₜ was the cumulative BMP-2 released at time t, and M_{∞} was the cumulative BMP-2 released at the end of the experiment, when a plateau in protein release had been reached. The effective BMP-2 diffusivity of the no affibody hydrogel was unaffected by the different media for release, likely because PEG demonstrates limited protein adsorption, resulting in nonspecific BMP-2 adsorption into the hydrogel.¹⁰⁶ Conversely, the effective BMP-2 diffusivity of the high-affinity affibody (SEQ ID NO: 1) hydrogel was higher in serum compared to saline (FIG. 12E). The low-affinity hydrogel (SEQ ID NO: 3) did not demonstrate significantly different BMP-2 release compared to any other group. Increased BMP-2 release in serum compared to saline may be due to the presence of proteases that may have affected protein binding and stability or lipids that may have affected the hydrophilic/lipophilic balance of the release media, potentially interfering with the hydrophobic interactions between the high-affinity affibody and the BMP-2.¹⁰⁷ However, since the low-affinity affibody (SEQ ID NO: 3) was predicted to interact with BMP-2 primarily via electrostatic interactions, these interactions may not have been largely affected by the components of serum. Although the diffusion rate of BMP-2 from hydrogels containing the high-affinity affibody was lower than that of hydrogels containing low-affinity affibodies in saline, there was no difference in cumulative BMP-2 release. Conversely, the presence of proteins and lipids in serum-containing release media introduced additional protein-protein and protein-lipid interactions, which significantly increased the diffusion rate of BMP-2 from hydrogels containing the high affinity-affibody. This result demonstrates that, depending on the nature of the affinity interaction, the presence of serum in the release media can increase the dissociation constant of the interaction and shift the equilibrium concentrations of bound and unbound protein, in turn increasing cumulative protein release. In saline alone, these interactions would not be affected.

### Example 12: BMP-2 Bioactivity Upon Release from Affibody-Conjugated PEG-Mal Hydrogels

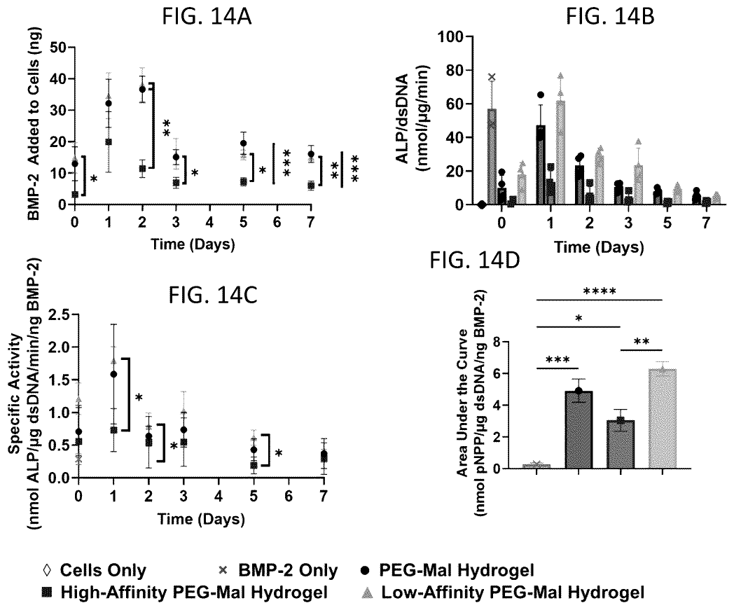

To determine whether BMP-2 bioactivity was preserved upon release from affibody-conjugated PEG-Mal hydrogels, ALP activity assays were performed on C2C12 cells using BMP-2 released from the hydrogels over a 7-day period. 200 μL 5 w/v % PEG-Mal hydrogels containing no affibody, low-affinity affibodies, or high-affinity affibodies were loaded with 200 ng of BMP-2. The hydrogels were then submerged in 1 mL of low serum media, and aliquots were taken immediately and after 1, 2, 3, 5, and 7 days. Fresh media was replenished at each timepoint. The BMP-2 content of each aliquot was quantified using ELISA (FIG. 14A). To evaluate ALP induction, C2C12 cells were seeded as described above, resuspended in 180 μL of the aliquots containing released BMP-2 from each timepoint, and incubated for 72 hours in 37° C. ALP activity was quantified, normalized to dsDNA, and plotted as a function of time (FIG. 14B).

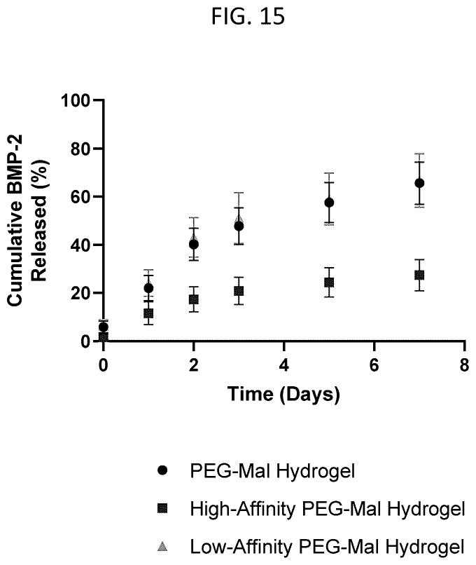

Initially, the high-affinity affibody (SEQ ID NO: 1) hydrogels bound more BMP-2, reducing the amount of BMP-2 present in solution at 0 hours compared to the no affibody and the low-affinity affibody (SEQ ID NO: 3) hydrogels (FIG. 14A); these data reflect the encapsulation efficiency results (FIG. 12E), which demonstrate that high-affinity affibody hydrogels encapsulated more BMP-2. As such, the no affibody and low-affinity affibody hydrogels released between 10-20 ng of BMP-2 at 0 hours compared to the high-affinity affibody hydrogels, which released less than 5 ng of BMP-2. At all the timepoints, the high-affinity affibody hydrogels released significantly less BMP-2 than the no affibody and low-affinity affibody hydrogels. Consequently, the ALP activity induced by BMP-2 released from the high-affinity affibody hydrogels was lower than that of the no affibody and low-affinity affibody hydrogels. Cumulative BMP-2 release was also quantified (FIG. 15), which demonstrated a similar release profile to the BMP-2 release into 10% serum (FIG. 12D), indicating that the inclusion of serum, even at low concentrations, affects the protein release kinetics of the affibody-conjugated hydrogels.

ALP activity was normalized to the amount of BMP-2 released at each timepoint for each hydrogel to determine if the osteogenic function of BMP-2 changed over time (FIG. 14C). Overall, the specific bioactivity of the BMP-2 decreased over time, which was likely due to denaturation of the protein at 37° C.¹⁰⁸ To determine the overall osteogenic effect of the BMP-2 over time, the area under the curve of the normalized ALP activity was calculated (FIG. 14D). The use of any hydrogel delivery vehicle increased and prolonged the total effect of the BMP-2 on the C2C12 cells, consistent with other studies.^{21,109} The use of the high-affinity affibody hydrogels resulted in lower total BMP-2 activity, which may be due to the extended binding of the BMP-2 to the affibodies and retention within the hydrogels. The early release of BMP-2 from the no affibody hydrogel and low-affinity affibody hydrogel resulted in more bioactive BMP-2 compared to the high-affinity affibody hydrogels, leading to higher overall BMP-2 activity. While some other affinity peptides and extracellular matrix molecules such as heparin have demonstrated a stabilizing effect on their target proteins,⁴⁰ this affibody-conjugated hydrogels did not exhibit this capacity with BMP-2 and C2C12 cells. One exemplary advantage of this approach is that the binding interactions of affibody-containing hydrogels are specific and tunable, making it easier to engineer materials that specifically perform their intended functions.

### Example 13: Hyaluronic Acid Hydrogels

This example describes hyaluronic acid (HA) hydrogels that can include one or more different affibodies provided herein, such as one or more of those in Table 1, to control release of proteins that correspond to the affibodies. Although use of a BMP-2 affibody is described, other affibodies can be used.

Materials: Sodium Hyaluronate (HA, 40 kDa and 100 kDa) were from Lifecore Biomedical LLC (Chaska, MN). Adipic acid dihydrazide (ADH) was from Spectrum chemical (Gardena, CA). Hydroxybenzotriazole (HOBt) was from Chem Impex (Wood Dale, Il). 1-Ethyl-3-(3-dimethylaminopropyl)carbodiimide (EDC) was from G Biosciences (St. Louis, MO). Sodium Periodate, Tert-butyl alcohol (TBA-OH), DMSO, 4-Dimethylaminopyridine (DMAP), and 5-Norbornene-2-carboxylic acid were from Sigma Aldrich (St. Louis, MO).

Synthesis of Adipic Acid Dihydrazide HA (ADH-HA): HA (200 mg, 0.527 mmol) was dissolved in 20 mL of diH20 to form a 1% w/v solution. Adipic dihydrazide (183.75 mg, 1.05 mmol) and hydroxy-benzotriazole (142.53 mg, 1.05 mmol) were added to the HA solution, adjusting the pH to 4.75. EDC was added (91.00 mg, 0.47 mmol) and the pH was monitored and maintained at 4.75 for 4 Hrs using 1 M HCl and NaOH. The solution was stirred for 24 hrs at room temperature, and dialyzed in 0.1 M NaCl in water for 2 days followed by diH2O for 2 days. The solution was sterile filtered and lyophilized.

Synthesis of Oxidized HA (HA-Ox): HA (200 mg, 0.561 mmol) was dissolved in 20 mL of diH20 to form a 1% w/v solution. Sodium periodate (322.00 mg, 0.281 mmol) was added to the solution and stirred overnight at room temperature protected from light. The reaction was quenched with 1 mL of propylene glycol and dialyzed with diH₂O for 3 days. The solution was sterile filtered and lyophilized.

Synthesis of Oxidized Norbornene HA Synthesis of Tert-butyl Alcohol HA (HA-TBA): HA (1.01 g, 2.506 mmol) was dissolved in 0.5 mL diH2O to form a 2% w/v solution. Dowex® MB Mixed Ion Exchange Resin (3.03 g) was added to the reaction and allowed to stir at room temperature overnight. The resin was vacuum filtered and the filtrate was then titrated to pH of X with TBA-OH and dialyzed in diH₂O over 3 days. The solution was sterile filtered and lyophilized.

Synthesis of Norbornene HA (Nor-HA): HA-TBA (153 mg, 0.35 mmol) was dissolved in 0.75 mL DMSO to form a 2% w/v solution. The flask was purged with N2 for 5 minutes then DMAP (145 mg, 1.049 mmol) was added to the reaction flask. Boc2O was added via syringe (32 uL, 0.14 mmol). The solution was stirred at 45° C. overnight then quenched with cold diH2O (10 mL). Nor-HA was precipitated from the solution by adding cold acetone (30 mL) then filtered and dialyzed in diH2o for 3 days. The solution was sterile filtered and lyophilized.

Oxidization of Norbornene HA (Nor-Ox): Nor-HA (91.75 mg, 0.18 mmol) was dissolved in diH2O to form a 1% w/v solution. Sodium periodate (10.57 mg, 0.049 mmol) was dissolved in diH₂O to 0.5M and added to the HA solution. The solution stirred overnight at room temperature protected from light. The reaction was quenched with 1 mL of propylene glycol and dialyzed with RO water for 3 days. The solution was sterile filtered and lyophilized.

Affibody Bioconjugation: NorOx-HA (14.5 mg) was dissolved in 1500 μL of 0.71 mg/mL high-affinity BMP-2-specific affibody (SEQ ID NO: 1) dissolved in PBS (1.45×10−4 mmol). 15 μL of 10% w/v Irgacure 2595 in methanol was added to the reaction vial, stirred, and illuminated with 365 nm light for 10 minutes. The solution was dialyzed with HEPES buffer at pH 7.0 for 1 day and RO water for 2 days. The solution was sterile filtered and lyophilized.

Preparation of HA Hydrogels: Hydrogels were prepared by reconstituting ADH-HA and aldehyde-containing HA(HA-Ox, Nor-Ox, or Nor-Ox-Aff) in 1×PBS. Hydrogels were prepared by mixing 50 uL of each copolymer.

Physiochemical Characterization

Degree of Modification (DOM): The degree of chemical modification of ADH-HA, HA-TBA, Nor-HA, and Nor-Ox-Aff was quantified using Nuclear Magnetic Resonance spectroscopy (1H NMR, 500 Hz, Bruker USA). The degree of oxidation was determined using titration with hydroxylamine hydrochloride.

BMP-2 Controlled Release: Release profiles of BMP-2 were assessed from NorOx-Aff, NorOx, and Ox platforms. Modified hyaluronic acid hydrogels were loaded with 15 ng/mL of BMP-2 (2472 Affibody: 1 BMP-2) and incubated at 37° C. Hydrogels were then allowed to passively release BMP-2 into a 0.1% BSA in PBS solution with aliquots of the supernatant taken over 28 days. The supernatant was analyzed with enzyme-linked immunosorbent assay (ELISA Human BMP-2 DuoSet—R&D systems) to evaluate the concentration of BMP-2 release over time. To compare the release rates of each platform clearly, we analyzed the Fickian diffusion slope k. Mt is defined as the mass of drug released at time t divided by the mass of drug released over time. Mₜ/(M_{∞})=kt^{1/2}

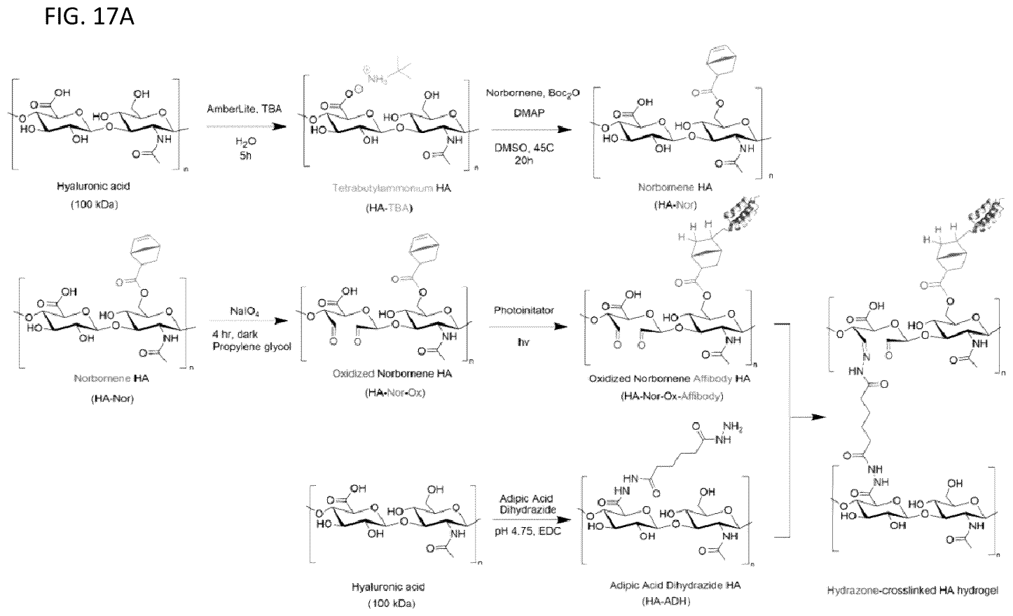

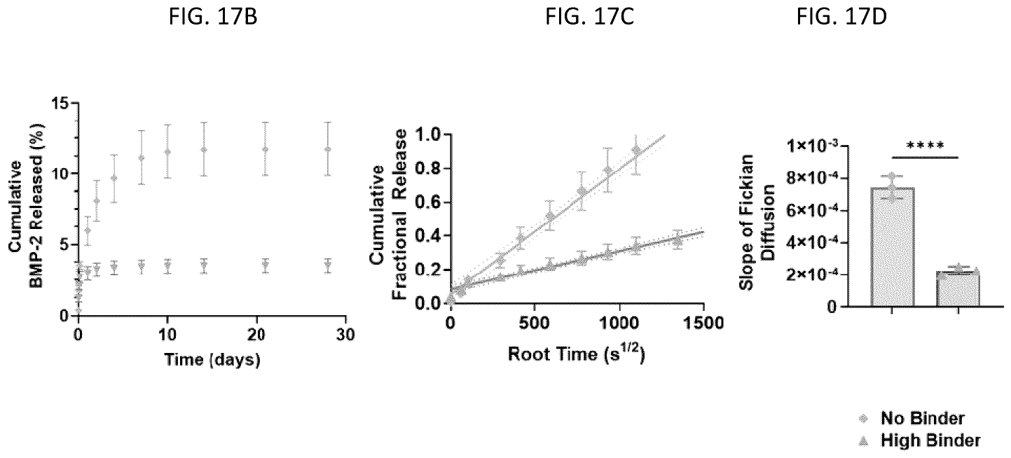

HA hydrogels containing a BMP-2 specific affibody (SEQ ID NO: 1) and BMP-2 protein were generated as shown in FIG. 17A. As shown in FIGS. 17B-17D, the amount of BMP-2 released from the hydrogel was lower with the affibody present, than without.

### Example 14: Identification of GM-CSF Affibodies

Using the methods described in Example 1, affibodies were identified for GM-CSF.

An initial yeast library expressing millions of randomized affibody variants underwent four cycles of magnetic-activated cell sorting to enrich the library for GM-CSF binders, followed by two cycles of fluorescence-activated cell sorting to isolate yeast populations that bind to GM-CSF. Individual yeast clones were sequenced, and their binding affinities were characterized.

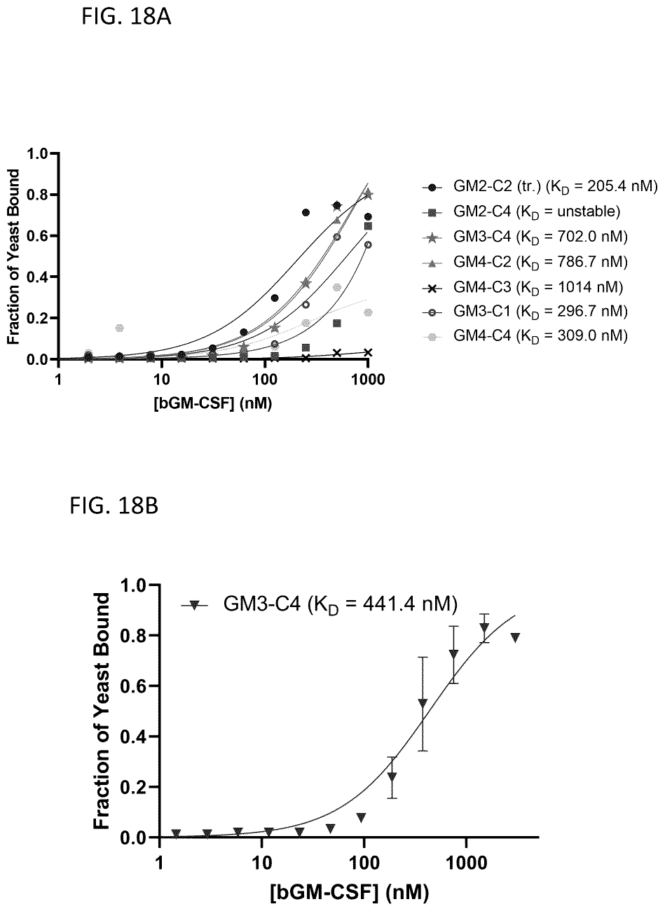

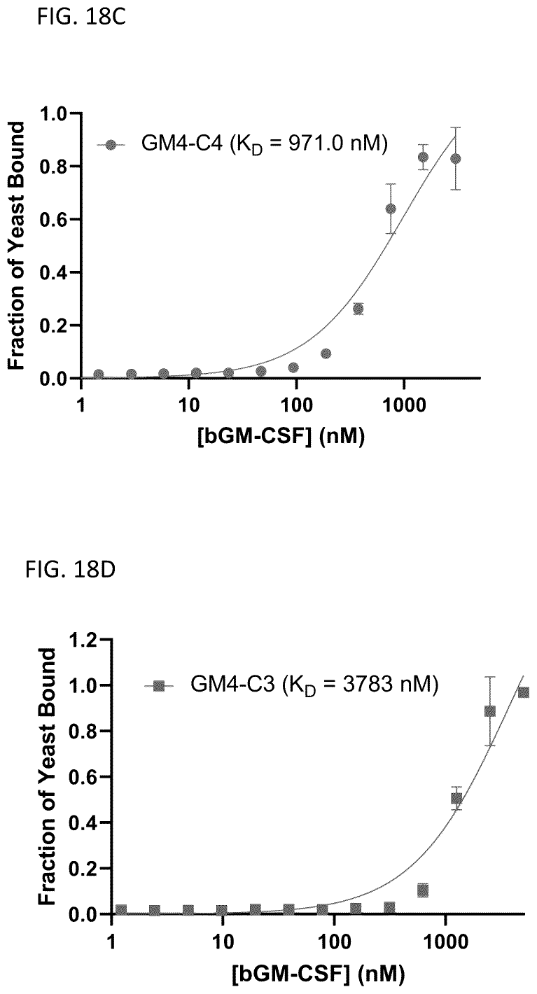

As shown in FIGS. 18A-18D, several affibodies specific for granulocyte macrophage colony-stimulating factor (GM-CSF) were identified (SEQ ID NOS: 12-19), having a dissociation constant of about 205.4 to 786.7 nM. Such affibodies can be used to control release of GM-CSF from a hydrogel. For example, such a hydrogel containing GM-CSF and one or more GM-CSF affibodies provided herein, can be used in the treatment of a wound (for example by applying the hydrogel to a wound or injury site).

### Example 15: Identification of Affibodies for Angiogenesis

Using the methods described in Example 1, affibodies were identified for proteins associated with angiogenesis.

A yeast surface display library containing approximately 800 million randomized affibody-encoding genes underwent magnetic- and fluorescence-activated cell sorts to isolate affibodies that bind specifically to VEGF, FGF-2, or PDGF. Monoclonal affibody-displaying yeast that exhibited binding to their protein target were isolated and sequenced. The affinities of surface-displayed affibodies for their target were estimated by incubating monoclonal yeast with 2.5-10000 nM of the target protein, followed by binding analysis using flow cytometry. Target specificity was evaluated by comparing monoclonal affibody binding between all three angiogenic proteins. Target-specific affibodies were transformed into E. coli and expressed with a hexahistidine tag and C-terminal cysteine (e.g., aa 59-65 of SEQ ID NO: 71) for purification and chemical conjugation, respectively.

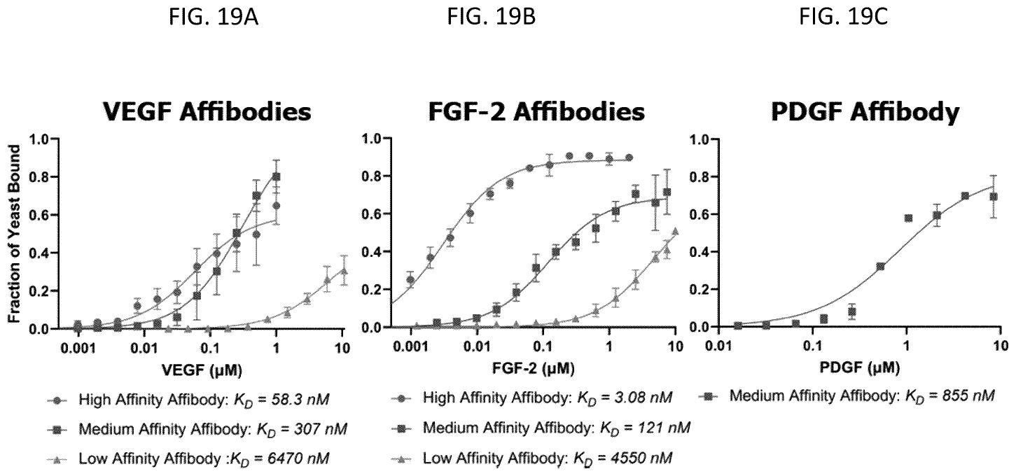

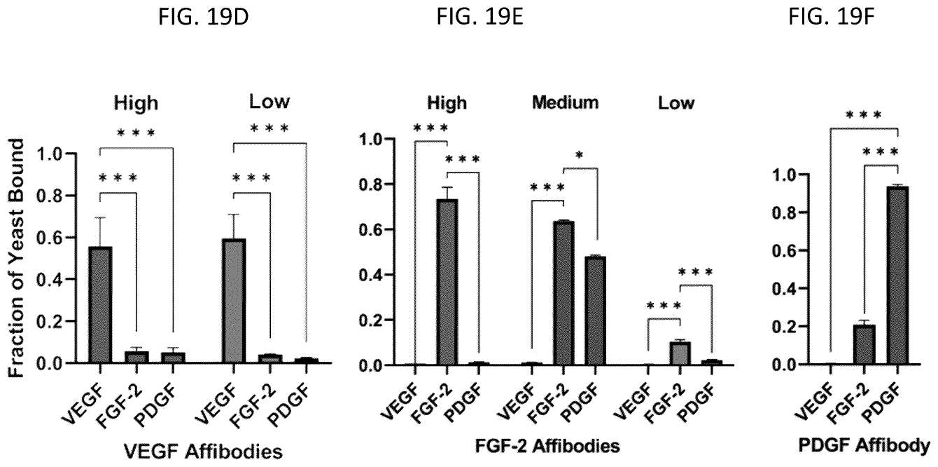

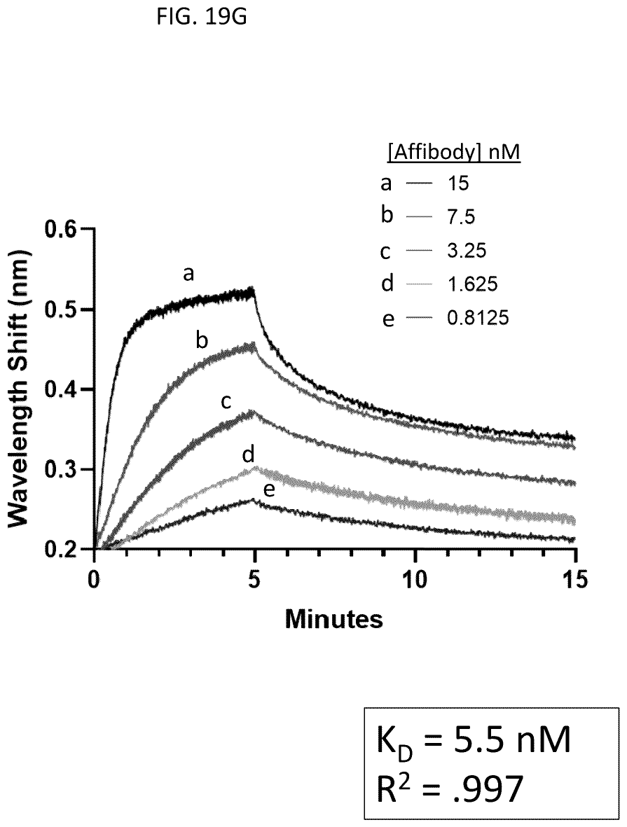

As shown in FIGS. 19A-19G, affibodies for VEGF, FGF-2 and PDGF were identified.

Affibodies with high (K_{D}=58.3±32.6 nM; SEQ ID NO: 20), medium (K_{D}=307 nM; SEQ ID NO: 21), and low (K_{D}=6470 nM; SEQ ID NO: 22) affinities for VEGF were identified; all exhibited specific binding to VEGF with negligible binding to FGF-2/PDGF. Additional VEGF affibodies were identified and are shown in SEQ ID NOS: 23-41.

Affibodies with high (K_{D}=3.08±0.45 nM; SEQ ID NO: 42), medium (K_{D}=121.2±36.7 nM; SEQ ID NO: 43), and low (K_{D}=4550 nM, SEQ ID NO: 44) affinities for FGF-2 were identified; the high-affinity affibody bound specifically to FGF-2, while the medium-affinity affibody exhibited binding to FGF-2 and PDGF. Additional FGF-2 affibodies were identified and are shown in SEQ ID NOS: 45-56.

One affibody with medium (K_{D}=855 nM; SEQ ID NO: 60) affinity for PDGF was identified, which bound strongly to PDGF and weakly to FGF-2. Characterization of a high-affinity PDGF affibody is shown in FIG. 19G. Additional PDGF affibodies were identified and are shown in SEQ ID NOS: 57-59.

### Example 16: Identification of Affibodies for Immune Regulation

Using the methods described in Example 1, affibodies were identified for IL-4, associated with inflammatory responses.

A yeast surface display library containing approximately 800 million randomized affibody-encoding genes underwent magnetic- and fluorescence-activated cell sorts to isolate affibodies that bind specifically to IL-4. Monoclonal affibody-displaying yeast that exhibited binding to their protein target were isolated and sequenced. The affinities of surface-displayed affibodies for their target were estimated by incubating monoclonal yeast with 2.5-10000 nM of the target protein, followed by binding analysis using flow cytometry. Target specificity was evaluated by comparing monoclonal affibody binding between all three angiogenic proteins. Target-specific affibodies were transformed into E. coli and expressed with a hexahistidine tag and C-terminal cysteine for purification and chemical conjugation, respectively.

Affibodies with high (K_{D}=4 nM; SEQ ID NO: 61) and low K_{D}=92,000 nM; SEQ ID NO: 62 affinities for IL-4 were identified; all exhibited specific binding to IL-4. Additional IL-4 affibodies were identified and are shown in SEQ ID NOS: 63-64. Such affibodies can be used to manipulate the immune response such as increase or decrease the recruitment and differentiation of immune cells.

### Example 17: Identification of Affibodies for Neural Survival

Using the methods described in Example 1, affibodies were identified for GDNF, associated with promoting survival and proliferation of neurons.

A yeast surface display library containing approximately 800 million randomized affibody-encoding genes underwent magnetic- and fluorescence-activated cell sorts to isolate affibodies that bind specifically to GDNF. Monoclonal affibody-displaying yeast that exhibited binding to their protein target were isolated and sequenced. The affinities of surface-displayed affibodies for their target were estimated by incubating monoclonal yeast with 2.5-10000 nM of the target protein, followed by binding analysis using flow cytometry. Target specificity was evaluated by comparing monoclonal affibody binding between all three angiogenic proteins. Target-specific affibodies were transformed into E. coli and expressed with a hexahistidine tag and C-terminal cysteine for purification and chemical conjugation, respectively.

Affibodies with high (K_{D}=1.7 nM and 5. nM; SEQ ID NO: 58, 59) and medium (K_{D}=855 nM; SEQ ID NO: 60) affinities for GDNF were identified. Additional GDNF affibodies were identified and are shown in SEQ ID NOS: 57.

### Example 18: Hyaluronic Acid and Alginate Hydrogels

This example describes hyaluronic acid (HA) and alginate hydrogels that can include one or more different affibodies provided herein, such as one or more of those in Table 1, to control release of proteins that correspond to the affibodies. Although use of a GDNF affibody is described, other affibodies can be used.

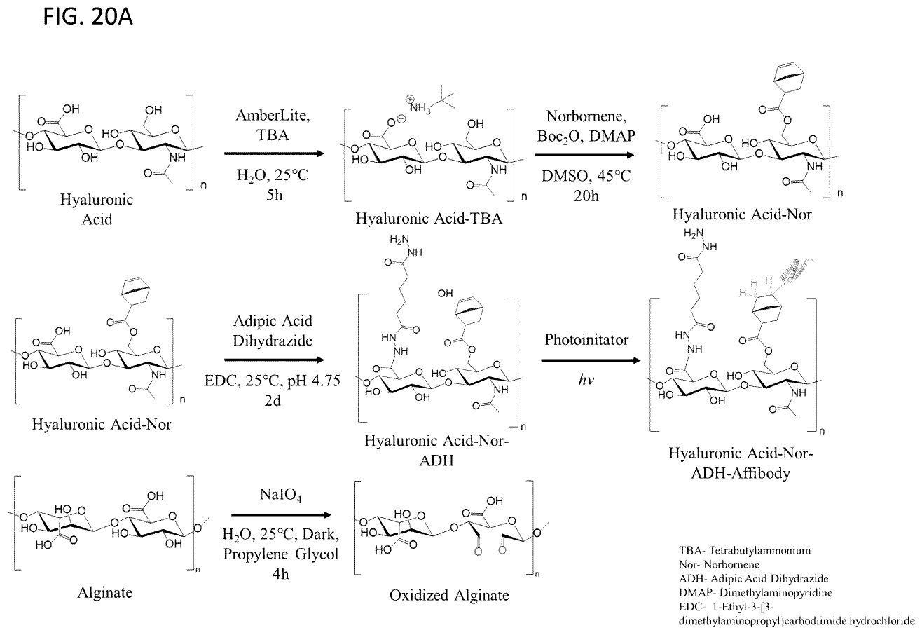

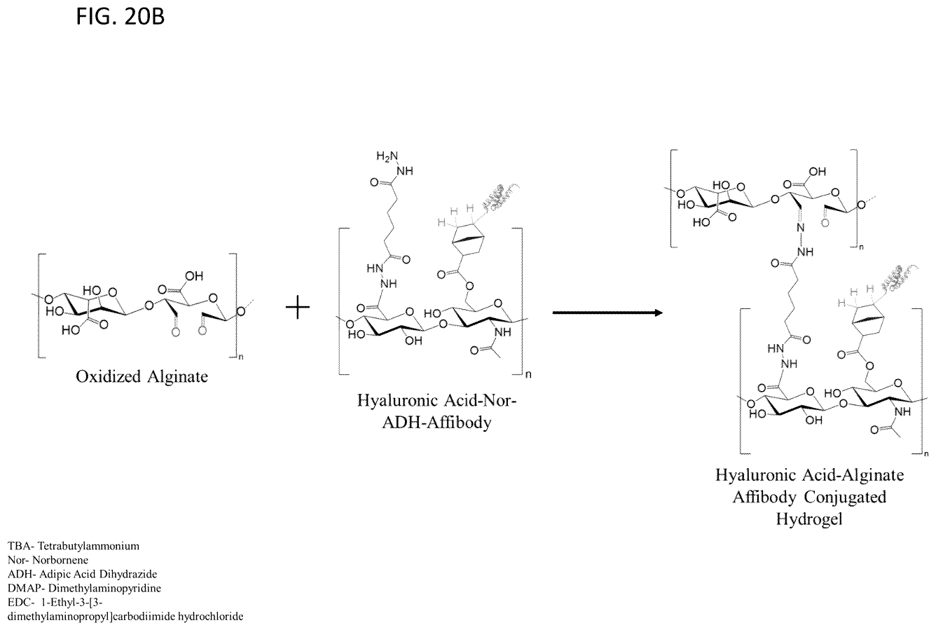

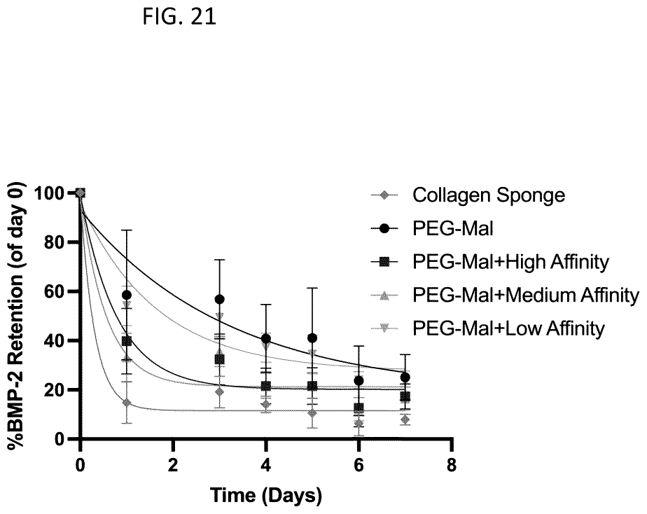

An ion exchange with hyaluronic acid (HA) using AmberLite for 5 h is performed, filtrated, and titrated with tetrabutylammonium (TBA) hydroxide until a pH of 7. The intermediate product obtained by the ion exchange will allow for the functionalization of HA with a norbornene (Nor) functional group which is used to bioconjugate glial cell-line derived neurotrophic factor-specific affibodies to the backbone of HA. BoC₂O activated coupling is performed with a norbornene in the presence of dimethylaminopyridine (DMAP). After, HA-Nor will be modified a second time to contain an adipic acid dihydrazide functional group for crosslinking with alginate. This is performed by adding ADH in the presence of EDC at a pH of 4.75. Once modified with ADH, the HA-Nor-ADH is used to bioconjugate GDNF-specific affibodies to the backbone of HA. This is performed through the addition of the affibody at a 2-molar excess and a photoinitiator, Irgacure 2959, and exposed to light at 365 nm for 30 m. Alginate is oxidized through the addition of NaIO4 to expose aldehydes that will be used to crosslink with ADH (FIGS. 20A-20B).

After both HA and alginate are modified with GDNF-specific affibodies and functional groups for crosslinking, each polymer will be dissolved in PBS and filter sterilized. Recombinant human GDNF will then be added to the HA solution to bind to the GDNF-specific affibodies and then loaded into a syringe. The oxidized alginate solution will be loaded into a separate syringe. The solutions will then be mixed by using a female-to-female luer lock and collected into a single syringe. The hydrogel mixture will be allowed to gel before being used in a rat spinal cord hemisection model (e.g., see Example 23).

### Example 19: In Vivo Visualization of Protein-Loaded Affibody-Conjugated Hydrogels

This example describes in vivo methods used to visualize the hydrogels of the present application, including those that contain protein(s) and a corresponding one or more protein-specific affibodies (such as one or more of those in Table 1). Although use of BMP-2 affibodies are described, other affibodies can be used.

The release of proteins from affibody-conjugated hydrogels in vivo can be tracked using fluorescently-labeled proteins. BMP-2 (R&D Biosystems) was fluorescently labeled with NIR 800CW dye (LICOR) per the manufacturer's instructions and purified and sterile filtered. Implantable PEG-Mal hydrogels were synthesized on 8 mm diameter absorbable collagen sponge (Medtronic inFUSE) scaffolds for mechanical support. 30 μL of 12.5 (w/v %) 4-arm PEG-Maleimide (Laysan Bio) in PBS pH 6.9 can be mixed with 30 μL of PBS pH 6.9 or 30 μL of PBS pH 6.9 containing 1.92 nmol of high-(SEQ ID NO: 1), medium—(SEQ ID NO: 2), or low-affinity (SEQ ID NO: 3) BMP-2 affibody and rotated for 30 minutes to form PEG-Mal-affibody intermediates. 60 μL of intermediate solution was added drop-wise onto the collage sponge and allowed to soak and absorb completely. 40 μL of 1.93 mg/mL dithiothreitol (DTT; GoldBio) in PBS pH 6.9 was added drop-wise to each hydrogel to crosslink the PEG-Mal intermediate solutions and form mechanically supported, affibody-conjugated PEG-Mal hydrogels. Hydrogels were washed with 500 μL of Dulbecco's PBS twice to remove unbound DTT and affibodies. Hydrogels can be loaded with 2.5 μg of fluorescently labeled BMP-2 and allowed to absorb for 2 hours away from light. A collagen only control was used, where an 8 mm diameter absorbable collagen sponge was soaked with the 2.5 μg solution of fluorescent BMP-2 for 2 hours away from light. The hydrogels and collagen sponges were placed in a sterile storage (well plate) and brought to the surgical suite away from light. All solutions were sterile-filtered using 0.2 μm syringe filters. All preparations can be performed in a sterile biological safety cabinet using aseptic technique.

In preparation for surgery, male 6-week-old Sprague Dawley rats (Charles River Laboratory) were anesthetized by isoflurane, administered buprenorphine (1 mg/kg), shaved along the back, and cleaned using isopropyl alcohol and chlorohexiderm. The rats were transferred to the surgical table for surgery. Longitudinal implants lateral to the spine made, and subcutaneous pockets formed with blunt dissection tools. Hydrogels and collagen sponges were implanted within the subcutaneous pockets. The incision sites were closed by wound clips or absorbable 4-0 suture material.

Fluorescent signals of the implants were visualized and quantified using Spectra In Vivo Imaging System (IVIS; Beckman Coulter). Rats were anesthetized by isoflurane and imaged using an overlay of photography and fluorescence imaging modalities where the fluorescence excitation and emission signals were 745 nm and 800 nm, respectively. Images were taken for 7 days. Signal associated with the fluorescent region of interest was normalized to the starting fluorescent signal.

As shown in FIG. 20, fluorescent BMP-2 was retained in the subcutaneous space in vivo, when affibodies were present. This demonstrates that the disclosed compositions can be used to control the release of therapeutic proteins.

### Example 20: Controlled Co-Delivery of BMP-2 and IL-4 from Dual-Affibody-Conjugated PEG-Maleimide Hydrogels

This example describes methods used to control delivery of two different proteins from a single hydrogel, using two protein-specific affibodies (such as one or more of those in Table 1). Although use of IL4- and BMP-2 affibodies are described, other combinations of affibodies can be used.

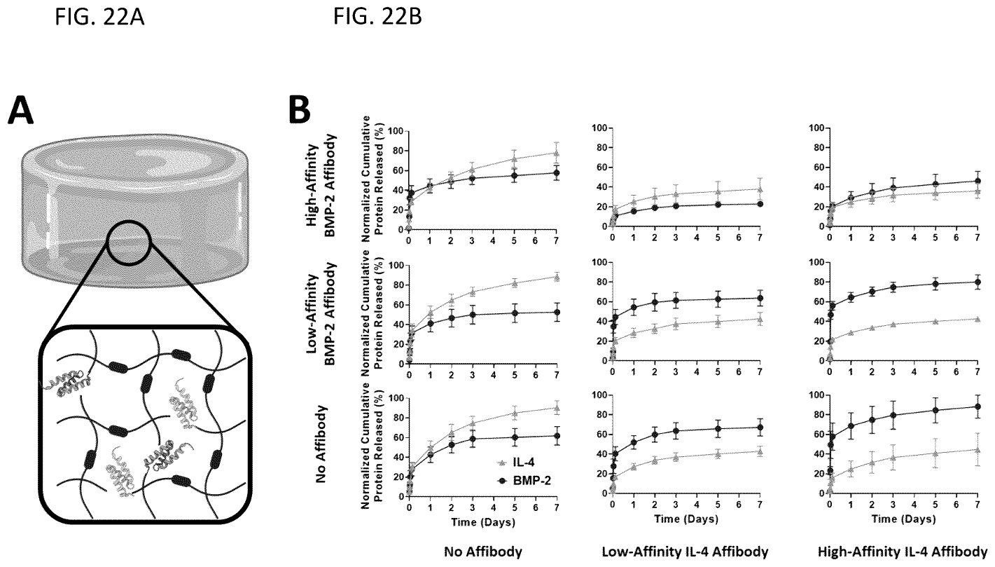

PEG-Mal hydrogels conjugated with no affibody, high- or low-affinity BMP-2 affibody (SEQ ID NOS: 1 and 3) and/or high- or low-affinity IL-4 affibody (SEQ ID NOS: 61 and 62) were synthesized as described herein. Briefly, 4-arm PEG-Mal was mixed with no affibody, high- or low-affinity BMP-2 affibody and/or high- or low-affinity IL-4 affibody to form affibody-conjugated intermediate solutions, and then crosslinked with DTT to form affibody-conjugated hydrogels (FIG. 22A). Hydrogels were loaded with 50 ng each of BMP-2 and IL-4 and aliquots were taken over 7 days. BMP-2 and IL-4 release was quantified by protein-specific ELISA. As shown in FIG. 22B, the additional of BMP-2 and IL-4 affibodies reduced the release of their respective proteins, such that the higher affinity affibodies reduced protein release more than lower affinity affibodies.

### Example 21: Encapsulation and Controlled Release of FGF-2 from Affibody-Conjugated PEG-Mal Hydrogels

This example describes PEG-Mal hydrogels containing FGF-2 affibodies, and measuring release of FGF-2 proteins from the hydrogels. Although use of FGF-2 affibodies are described, other affibodies can be used.

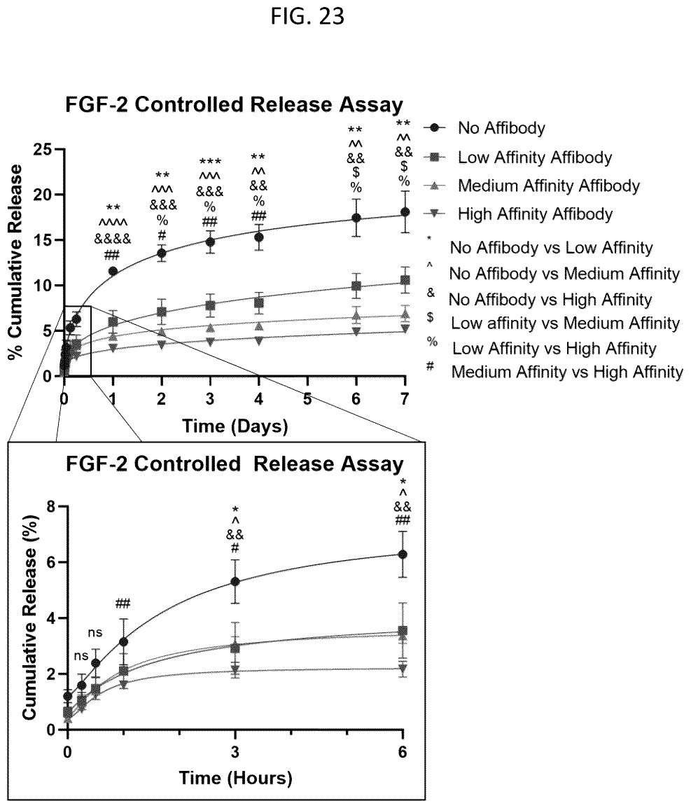

PEG-Mal hydrogels conjugated with no affibody, high-, medium- or low-affinity FGF-2 affibody were synthesized as described in Example 10. Briefly, 4-arm PEG-Mal was mixed with no affibody, high-, medium-, or low-affinity BMP-2 affibody to form affibody-conjugated intermediate solutions, and then crosslinked with DTT to form affibody-conjugated hydrogels. Hydrogels were loaded with 100 ng FGF-2 and aliquots were taken over 7 days. FGF-2 release was quantified by protein-specific ELISA. As shown in FIG. 23, release of FGF-2 from the affibody-conjugated PEG-Mal hydrogels depended on the K_{D} of the affibody used. The inclusion of affibodies reduced the release of FGF-2, such that higher affinity affibodies resulted in less release of FGF-2 than lower affinity affibodies.

### Example 22: Encapsulation and Controlled Release of PDGF from Affibody-Conjugated PEG-Mal Hydrogels

This example describes PEG-Mal hydrogels containing PDGF affibodies, and measuring release of PDGF proteins from the hydrogels. Although use of FGF-2 affibodies are described, other affibodies can be used.

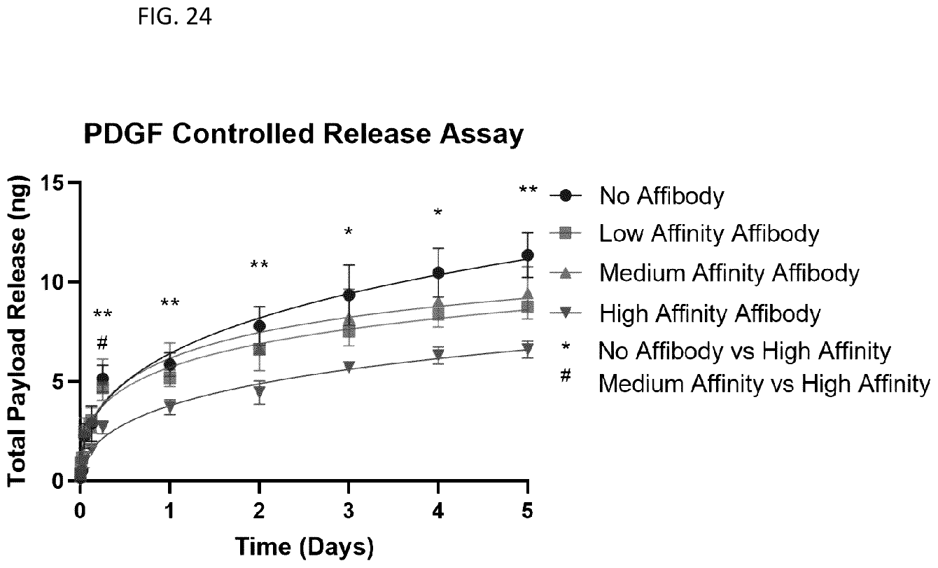

PEG-Mal hydrogels conjugated with no affibody, high-, medium- or low-affinity PDGF affibody were synthesized as described in Example 10. Briefly, 4-arm PEG-Mal was mixed with no affibody, high-, medium-, or low-affinity BMP-2 affibody to form affibody-conjugated intermediate solutions, and then crosslinked with DTT to form affibody-conjugated hydrogels. Hydrogels were loaded with 100 ng PDGF and aliquots were taken over 7 days. PDGF release was quantified by protein-specific ELISA. As shown in FIG. 24, release of PDGF from the affibody-conjugated PEG-Mal hydrogels depended on the K_{D} of the affibody used. The inclusion of affibodies reduced the release of PDGF, such that higher affinity affibodies resulted in less release of PDGF than lower affinity affibodies.

### Example 23: Treatment of Spinal Cord Injury

This example describes methods of using a hydrogel containing one or more GDNF-specific affibodies (such as one or more of those in Table 1) and GDNF, to control release of GDNF to treat a spinal cord injury in vivo.

A hemisection rat spinal cord injury model can be employed. Long Evans rats will be anesthetized with vaporized isoflurane. A laminectomy at T9-T10 will be used to expose the spinal cord and a 4 mm lateral hemisection defect will be performed on the left side of the spinal cord (De Laporte et al., Molecular Therapy, 17(2), 318-326, 2009). The hydrogels (e.g., see Example 18) with and without one or more GDNF affibodies (and with GDNF protein) will be injected into the defect region. After, the muscles will be sutured closed, and the skin will be stapled. Post-operative care will include antibiotics, pain medication, and sodium lactate solution. Bladders will be expressed twice daily until function is recovered. Functional recovery of the rats with hydrogels containing one or more GDNF affibodies and without affibodies will be evaluated over 6 weeks.

### Example 24: Treatment of Bone Defects

This example describes methods that can be used with a hydrogel that includes one or more BMP-2 affibodies (e.g., one or more of SEQ ID NOS: 1-11) and BMP-2.

A rat model of an 8-mm critically sized femoral bone defect can be used to evaluate the effect of a hydrogel that includes one or more BMP-2 affibodies (e.g., one or more of SEQ ID NOS: 1-11) and BMP-2 on functional bone formation. Femurs will be stabilized with a polysulfone fixation plate with metal risers prior to creation of the defect. Similar to previous studies, a polycaprolactone (PCL) mesh tube with laser-cut holes will be placed in the defect site, and hydrogels will be injected into the tube. Hydrogels (such as those described herein) can be fabricated with 1) no BMP-2 or affibody binding partners, 2) BMP-2, or 3) BMP-2 and one or more BMP-2 affibodies.

The amount of BMP-2 affibodies required for BMP-2 localization can be determined based on in vitro experiments. A sample size of at least 10 defects per group can be used to evaluate bone repair via x-ray radiography, micro-computed tomography, and torsion testing for mechanical strength. Histology will also be performed to evaluate morphology of regenerated tissue in the bone defect.

### Example 25: Methods of Treating Vascular Disease

The rat critical hind limb ischemia model can be used to demonstrate the effects of sequential release of angiogenic growth factors (e.g., VEGF, FGF-2, and/or PDGF) from a biomaterial delivery vehicle on functional neovascularization. Briefly, the superficial femoral artery and vein will be ligated near the proximal and distal ends, with excision of the vessel tissue between the ligation points, to produce ischemic conditions in the lower hind limb. A polycaprolactone (PCL) mesh tube with laser-cut holes will be placed within the ischemic tissue, and hydrogels will be injected into the tube. Hydrogels will be fabricated with 1) no growth factors or affibody binding partners, 2) growth factors VEGF/FGF-2/PDGF, or 3) growth factors and associated affibodies (e.g., one or more of those in Table 1 for these proteins). Appropriate stoichiometric affibody:growth factor ratios for desired growth factor localization and release kinetics will be determined based on in vitro experiments. Capillary and vessel formation will be evaluated using X-ray microangiography, microcomputed tomography (micro-CT), and histology.

### Example 26: Exemplary Hydrogels

This example provides exemplary hydrogels that include HA or PEG. Although incorporation of BMP-2 affibodies is described, other affibodies such as those in Table 1 can also be used.

### Hyaluronic Acid Hydrogels

In some examples a hyaluronic acid hydrogel is administered by injection into a subject, for example subcutaneously at an injury or disease site.

### HA Solutions:

Weigh out ˜20±0.01 mg of modified HA polymer (Modified w/Aldehydes or Hydrazides).
Add to a 15 mL conical vial with (2000 uL) 1×PBS solution—this will generate a 1% polymer content.
Rotate overnight at room temp. for polymer to fully dissolve.
If you want a more concentrated stock solution and use dilutions for lower weight percent—add ˜20±0.01 mg to 889 uL of 1×PBS solution to make a 2.25% polymer content.
   Add 1:1 volumes Hydrazide:Aldehyde of desired w/v % combinations to form a hydrogel. (See table below for combinations)

$$\vert \frac{Polymer weight ( mg )}{Volume of Solution ( uL )} \times 100 \% = \frac{w}{v} \%$$

| Sample | ADH w/v % | Ox w/v % |
|---|---|---|
| 1 | 0.5 | 0.5 |
| 2 | 2.25 | 0.5 |
| 3 | 0.5 | 2.25 |
| 4 | 2.25 | 2.25 |
| 5 | 1.75 | 1.75 |
| 6 (MF) | 2.22 | 1.83 |

### Gel Mixing and Dilution:

For a 50 uL aliquot of 1% solution (via dilution route): add 23 uL of 2.25% polymer solution to a small centrifuge vial.
Dilute with 27 uL of 1×PBS solution.
Pipette 50 uL of Aldehyde HA (ex. Ox) into a new centrifuge tube, then add 50 uL of Hydrazide HA (ex. ADH). Gel formation should occur within 45 s for 2.25%:2.25% mixtures and ˜ 3 min for 1%:1% mixtures.
   C₁V₁=C₂V₂
(2.25%)(x)=(1.00%)(50 uL) x=23 uL (of the 2.25% sol'n)50 uL−23 uL=27 uL 1×PBS

### Affibody Formulations:

Modified HA polymers (Modified w/Aldehydes+Norbornene=NorOx, or Aldehydes+Methacrylate=MeOx) are combined with affibodies (e.g., SEQ ID NOS: 1, 2 and 3) to form bioconjugate polymers.

NorOx-HA (1 w/v %) with Desired Affinity BMP-2 (or other protein) Affibody Polymer
100 mg NorOx-HA dissolved in 1×PBS
15 mg Affibody
50 uL 10% Irgacure 2959
30 minutes of 365 nm light MeOx-HA (1 w/v %) with Desired Affinity BMP-2 (or other protein) Affibody Polymer
100 mg MeOx-HA dissolved in 1×PBS
15 mg Affibody
50 uL 10% Irgacure 2959
30 minutes of 365 nm light

### PEG Hydrogels

In some examples a PEG hydrogel is administered surgically, for example implanted at an injury or disease site.

### Materials

4-Arm PEG-Maleimide (20 kDa) (Laysan Bio) (PEG-MAL)
Dithiothreitol (DTT) (GoldBio)
Soluble Affibodies, such as a high, moderate, and low affinity affibody specific for one target, such as BMP-2, GM-CSF, VEGF, FGF-2, IL-4, GDNF, or PDGF. An example combination is provided below. However, the hydrogel can include affibodies specific for two or more different proteins, such as 2, 3, 4, 5, 6 or all of BMP-2, GMCSF, VEGF, FGF-2, IL-4, GDNF, or PDGF.

High Affinity BMP-2 Affibody
(AEAKYYKEVSSAATQIRYLPNLTAFQKAAFYAALLDDPSQSSELLSEAK
KLNDSQAPKHHHHHHC; SEQ ID NO: 71)
Moderate Affinity BMP-2 Affibody
(AEAKYAKEQFNAYVVIFYLPNLTASQKAAFVDALSNDPSQSSELLSEAK
KLNDSQAPKHHHHHHC; SEQ ID NO: 72)
Low Affinity BMP-2 Affibody
(AEAKYYKEGDNAYNVIYGLPNLTRPQRLAFIVALENDPSQSSELLSEAK
KLNDSQAPKHHHHHHC; SEQ ID NO: 73)
High Affinity GM-CSF Affibody
(AEAKYTKELFNAVGEITALPNLTRYHLYAFYYALLNDPSQSSELLSEAK
KLNDSQAPKHHHHHHC; SEQ ID NO: 74)
PBS (pH 7)

### Formulations:

PEG Only (Control Gels): 5% (w/v)
40 uL of 12.5% PEG-MAL in PBS
10 uL PBS
50 uL 1.54 mg/mL DTT
   PEG-Mal (5% w/v) can include a high, moderate, and/or low affinity affibody specific for one target, such as BMP-2, GMCSF, VEGF, FGF-2, IL-4, GDNF, or PDGF. Examples are provided below. However, the hydrogel can include affibodies specific for two or more different proteins, such as 2, 3, 4, 5, 6 or all of BMP-2, GMCSF, VEGF, FGF-2, IL-4, GDNF, or PDGF.
   PEG-Mal (5% w/v) with High Affinity BMP-2 Affibody Gel
40 uL of 12.5% PEG-MAL in PBS
13.92 uL in 1 mg/mL High Aff BMP-2 Affibody in PBS
6.02 ul PBS
40 uL of 1.92 mg/mL
   PEG-Mal (5% w/v) with Mid Affinity BMP-2 Affibody Gel
40 uL of 12.5% PEG-MAL in PBS
13.999 uL in 1 mg/mL Mid Aff BMP-2 Affibody in PBS
6.005 ul PBS
40 uL of 1.92 mg/mL
   PEG-Mal (5% w/v) with Low Affinity BMP-2 Affibody Gel
40 uL of 12.5% PEG-MAL in PBS
14.26 uL in 1 mg/mL Low Aff BMP-2 Affibody in PBS
5.74 ul PBS
40 uL of 1.92 mg/mL
   PEG-Mal (5% w/v) with High Affinity GM-CSF Affibody Gel
30 uL of 16.666% PEG-MAL in PBS
26.01 uL in 1 mg/mL High Aff GM-CSF Affibody in PBS
3.99 ul PBS
40 uL of 1.92 mg/mL

### REFERENCES

(1) Miyazono, K.; Kamiya, Y.; Morikawa, M. Bone Morphogenetic Protein Receptors and Signal Transduction. J. Biochem. (Tokyo) 2010, 147 (1), 35-51. https://doi.org/10.1093/jb/mvp148.
(2) James, A. W.; LaChaud, G.; Shen, J.; Asatrian, G.; Nguyen, V.; Zhang, X.; Ting, K.; Soo, C. A Review of the Clinical Side Effects of Bone Morphogenetic Protein-2. Tissue Eng. Part B Rev. 2016, 22 (4), 284-297. https://doi.org/10.1089/ten.teb.2015.0357.
(3) Hiepen, C.; Benn, A.; Denkis, A.; Lukonin, I.; Weise, C.; Boergermann, J. H.; Knaus, P. BMP2-Induced Chemotaxis Requires PI3K P55γ/P110α-Dependent Phosphatidylinositol (3,4,5)-Triphosphate Production and LL5β Recruitment at the Cytocortex. BMC Biol. 2014, 12 (1), 43. https://doi.org/10.1186/1741-7007-12-43.
(4) Borgiani, E.; Duda, G. N.; Willie, B. M.; Checa, S. Bone Morphogenetic Protein 2-Induced Cellular Chemotaxis Drives Tissue Patterning during Critical-Sized Bone Defect Healing: An in Silico Study. Biomech. Model. Mechanobiol. 2021, 20 (4), 1627-1644. https://doi.org/10.1007/s10237-021-01466-0.
(5) Lind, M.; Eriksen, E. F.; Bünger, C. Bone Morphogenetic Protein-2 but Not Bone Morphogenetic Protein-4 and -6 Stimulates Chemotactic Migration of Human Osteoblasts, Human Marrow Osteoblasts, and U2-OS Cells. Bone 1996, 18 (1), 53-57. https://doi.org/10.1016/8756-3282(95)00423-8.
(6) SUN, J.; LI, J.; LI, C.; YU, Y. Role of Bone Morphogenetic Protein-2 in Osteogenic Differentiation of Mesenchymal Stem Cells. Mol. Med. Rep. 2015, 12 (3), 4230-4237. https://doi.org/10.3892/mmr.2015.3954.
(7) Briquez, P. S.; Tsai, H.-M.; Watkins, E. A.; Hubbell, J. A. Engineered Bridge Protein with Dual Affinity for Bone Morphogenetic Protein-2 and Collagen Enhances Bone Regeneration for Spinal Fusion. Sci. Adv. 2021, 7 (24), eabh4302. https://doi.org/10.1126/sciadv.abh4302.
(8) Kim, H. D.; Valentini, R. F. Retention and Activity of BMP-2 in Hyaluronic Acid-Based Scaffolds in Vitro. J. Biomed. Mater. Res. 2002, 59 (3), 573-584. https://doi.org/10.1002/jbm.10011.
(9) Noshi, T.; Yoshikawa, T.; Dohi, Y.; Ikeuchi, M.; Horiuchi, K.; Ichijima, K.; Sugimura, M.; Yonemasu, K.; Ohgushi, H. Recombinant Human Bone Morphogenetic Protein-2 Potentiates the in Vivo Osteogenic Ability of Marrow/Hydroxyapatite Composites. Artif Organs 2001, 25 (3), 201-208. https://doi.org/10.1046/j.1525-1594.2001.025003201.x.
(10) Kamal, A. F.; Siahaan, O. S. H.; Fiolin, J. Various Dosages of BMP-2 for Management of Massive Bone Defect in Sprague Dawley Rat. Arch. Bone Jt. Surg. 2019, 7 (6), 498-505.
(11) Hettiaratchi, M. H.; Rouse, T.; Chou, C.; Krishnan, L.; Stevens, H. Y.; Li, M.-T. A.; McDevitt, T. C.; Guldberg, R. E. Enhanced in Vivo Retention of Low Dose BMP-2 via Heparin Microparticle Delivery Does Not Accelerate Bone Healing in a Critically Sized Femoral Defect. Acta Biomater. 2017, 59, 21-32. https://doi.org/10.1016/j.actbio.2017.06.028.
(12) Rihn, J. A.; Patel, R.; Makda, J.; Hong, J.; Anderson, D. G.; Vaccaro, A. R.; Hilibrand, A. S.; Albert, T. J. Complications Associated with Single-Level Transforaminal Lumbar Interbody Fusion. Spine J. Off J. North Am. Spine Soc. 2009, 9 (8), 623-629. https://doi.org/10.1016/j.spinee.2009.04.004.
(13) Weber, L. M.; Lopez, C. G.; Anseth, K. S. The Effects of PEG Hydrogel Crosslinking Density on Protein Diffusion and Encapsulated Islet Survival and Function. J. Biomed. Mater. Res. A 2009, 90 (3), 720-729. https://doi.org/10.1002/jbm.a.32134.
(14) Sheth, S.; Barnard, E.; Hyatt, B.; Rathinam, M.; Zustiak, S. P. Predicting Drug Release From Degradable Hydrogels Using Fluorescence Correlation Spectroscopy and Mathematical Modeling. Front. Bioeng. Biotechnol. 2019, 7.
(15) Dorogin, J.; Townsend, J. M.; Hettiaratchi, M. H. Biomaterials for Protein Delivery for Complex Tissue Healing Responses. Biomater. Sci. 2021, 9 (7), 2339-2361. https://doi.org/10.1039/D0BM01804J.
(16) Hettiaratchi, M. H.; Chou, C.; Servies, N.; Smeekens, J. M.; Cheng, A.; Esancy, C.; Wu, R.; McDevitt, T. C.; Guldberg, R. E.; Krishnan, L. Competitive Protein Binding Influences Heparin-Based Modulation of Spatial Growth Factor Delivery for Bone Regeneration. Tissue Eng. Part A 2017, 23 (13-14), 683-695. https://doi.org/10.1089/ten.tea.2016.0507.
(17) Rajabi, S.; Jalili-Firoozinezhad, S.; Ashtiani, M. K.; Le Carrou, G.; Tajbakhsh, S.; Baharvand, H. Effect of Chemical Immobilization of SDF-1α into Muscle-Derived Scaffolds on Angiogenesis and Muscle Progenitor Recruitment. J. Tissue Eng. Regen. Med. 2018, 12 (1), e438-e450. https://doi.org/10.1002/term.2479.
(18) Bax, D. V.; Davidenko, N.; Hamaia, S. W.; Farndale, R. W.; Best, S. M.; Cameron, R. E. Impact of UV- and Carbodiimide-Based Crosslinking on the Integrin-Binding Properties of Collagen-Based Materials. Acta Biomater. 2019, 100, 280-291. https://doi.org/10.1016/j.actbio.2019.09.046.
(19) Lai, J.-Y.; Luo, L.-J.; Ma, D. Effect of Cross-Linking Density on the Structures and Properties of Carbodiimide-Treated Gelatin Matrices as Limbal Stem Cell Niches. Int. J. Mol. Sci. 2018, 19 (11), 3294. https://doi.org/10.3390/ijms19113294.
(20) Yamamoto, S.; Iwamaru, Y.; Shimizu, Y.; Ueda, Y.; Sato, M.; Yamaguchi, K.; Nakanishi, J. Epidermal Growth Factor-Nanoparticle Conjugates Change the Activity from Anti-Apoptotic to pro-Apoptotic at Membrane Rafts. Acta Biomater. 2019, 88, 383-391. https://doi.org/10.1016/j.actbio.2019.02.026.
(21) Hettiaratchi, M. H.; Miller, T.; Temenoff, J. S.; Guldberg, R. E.; McDevitt, T. C. Heparin Microparticle Effects on Presentation and Bioactivity of Bone Morphogenetic Protein-2. Biomaterials 2014, 35 (25), 7228-7238. https://doi.org/10.1016/j.biomaterials.2014.05.011.
(22) Benoit, D. S. W.; Durney, A. R.; Anseth, K. S. The Effect of Heparin-Functionalized PEG Hydrogels on Three-Dimensional Human Mesenchymal Stem Cell Osteogenic Differentiation. Biomaterials 2007, 28 (1), 66-77. https://doi.org/10.1016/j.biomaterials.2006.08.033.
(23) Sakiyama-Elbert, S. E.; Hubbell, J. A. Controlled Release of Nerve Growth Factor from a Heparin-Containing Fibrin-Based Cell Ingrowth Matrix. J. Controlled Release 2000, 69 (1), 149-158. https://doi.org/10.1016/S0168-3659(00)00296-0.
(24) Bhattacharya, D. S.; Svechkarev, D.; Bapat, A.; Patil, P.; Hollingsworth, M. A.; Mohs, A. M. Sulfation Modulates the Targeting Properties of Hyaluronic Acid to P-Selectin and CD44. ACS Biomater. Sci. Eng. 2020, 6 (6), 3585-3598. https://doi.org/10.1021/acsbiomaterials.0c00115.
(25) Limasale, Y. D. P.; Atallah, P.; Werner, C.; Freudenberg, U.; Zimmermann, R. Tuning the Local Availability of VEGF within Glycosaminoglycan-Based Hydrogels to Modulate Vascular Endothelial Cell Morphogenesis. Adv. Funct. Mater. 2020, 2000068. https://doi.org/10.1002/adfm.202000068.
(26) Abune, L.; Wang, Y. Affinity Hydrogels for Protein Delivery. Trends Pharmacol. Sci. 2021, 42 (4), 300-312. https://doi.org/10.1016/j.tips.2021.01.005.
(27) Wang, Y.; Lan, H.; Yin, T.; Zhang, X.; Huang, J.; Fu, H.; Huang, J.; McGinty, S.; Gao, H.; Wang, G.; Wang, Z. Covalent Immobilization of Biomolecules on Stent Materials through Mussel Adhesive Protein Coating to Form Biofunctional Films. Mater. Sci. Eng. C 2020, 106, 110187. https://doi.org/10.1016/j.msec.2019.110187.
(28) Mardilovich, A.; Craig, J. A.; McCammon, M. Q.; Garg, A.; Kokkoli, E. Design of a Novel Fibronectin-Mimetic Peptide-Amphiphile for Functionalized Biomaterials. Langmuir 2006, 22 (7), 3259-3264. https://doi.org/10.1021/la052756n.
(29) Crispim, J. F.; Fu, S. C.; Lee, Y. W.; Fernandes, H. A. M.; Jonkheijm, P.; Yung, P. S. H.; Saris, D. B. F. Bioactive Tape With BMP-2 Binding Peptides Captures Endogenous Growth Factors and Accelerates Healing After Anterior Cruciate Ligament Reconstruction. Am. J. Sports Med. 2018, 46 (12), 2905-2914. https://doi.org/10.1177/0363546518787507.
(30) Soontornworajit, B.; Zhou, J.; Shaw, M. T.; Fan, T.-H.; Wang, Y. Hydrogel Functionalization with DNA Aptamers for Sustained PDGF-BB Release. Chem. Commun. 2010, 46 (11), 1857-1859. https://doi.org/10.1039/B924909E.
(31) Hettiaratchi, M. H.; Krishnan, L.; Rouse, T.; Chou, C.; McDevitt, T. C.; Guldberg, R. E. Heparin-Mediated Delivery of Bone Morphogenetic Protein-2 Improves Spatial Localization of Bone Regeneration. Sci. Adv. 2020, 6 (1), eaay1240. https://doi.org/10.1126/sciadv.aay1240.
(32) Peysselon, F.; Ricard-Blum, S. Heparin-Protein Interactions: From Affinity and Kinetics to Biological Roles. Application to an Interaction Network Regulating Angiogenesis. Matrix Biol. 2014, 35, 73-81. https://doi.org/10.1016/j.matbio.2013.11.001.
(33) Shaughnessy, A. F. Monoclonal Antibodies: Magic Bullets with a Hefty Price Tag. BMJ 2012, 345 (dec12 1), e8346-e8346. https://doi.org/10.1136/bmj.e8346.
(34) Sexton, K.; Tichauer, K.; Samkoe, K. S.; Gunn, J.; Hoopes, P. J.; Pogue, B. W. Fluorescent Affibody Peptide Penetration in Glioma Margin Is Superior to Full Antibody. PLoS ONE 2013, 8 (4), e60390. https://doi.org/10.1371/journal.pone.0060390.
(35) Sachdeva, S.; Joo, H.; Tsai, J.; Jasti, B.; Li, X. A Rational Approach for Creating Peptides Mimicking Antibody Binding. Sci. Rep. 2019, 9 (1), 997. https://doi.org/10.1038/s41598-018-37201-6.
(36) Trier, N.; Hansen, P.; Houen, G. Peptides, Antibodies, Peptide Antibodies and More. Int. J. Mol. Sci. 2019, 20 (24), 6289. https://doi.org/10.3390/ijms20246289.
(37) Muñoz, E. M.; Linhardt, R. J. Heparin-Binding Domains in Vascular Biology. Arterioscler. Thromb. Vasc. Biol. 2004, 24 (9), 1549-1557. https://doi.org/10.1161/01.ATV.0000137189.22999.3f.
(38) Ren, C.; Wen, X.; Mencius, J.; Quan, S. Selection and Screening Strategies in Directed Evolution to Improve Protein Stability. Bioresour. Bioprocess. 2019, 6 (1), 53. https://doi.org/10.1186/s40643-019-0288-y.
(39) Teal, C. J.; Hettiaratchi, M. H.; Ho, M. T.; Ortin-Martinez, A.; Ganesh, A. N.; Pickering, A. J.; Golinski, A. W.; Hackel, B. J.; Wallace, V. A.; Shoichet, M. S. Directed Evolution Enables Simultaneous Controlled Release of Multiple Therapeutic Proteins from Biopolymer-Based Hydrogels. Adv. Mater. n/a (n/a), 2202612. https://doi.org/10.1002/adma.202202612.
(40) Bostock, C.; Teal, C. J.; Dang, M.; Golinski, A. W.; Hackel, B. J.; Shoichet, M. S. Affibody-Mediated Controlled Release of Fibroblast Growth Factor 2. J. Controlled Release 2022, 350, 815-828. https://doi.org/10.1016/j.jconrel.2022.09.004.
(41) Wang, J.; Youngblood, R.; Cassinotti, L.; Skoumal, M.; Corfas, G.; Shea, L. An Injectable PEG Hydrogel Controlling Neurotrophin-3 Release by Affinity Peptides. J. Controlled Release 2021, 330, 575-586. https://doi.org/10.1016/j.jconrel.2020.12.045.
(42) Löfblom, J.; Feldwisch, J.; Tolmachev, V.; Carlsson, J.; Stihl, S.; Frejd, F. Y. Affibody Molecules: Engineered Proteins for Therapeutic, Diagnostic and Biotechnological Applications. FEBS Lett. 2010, 584 (12), 2670-2680. https://doi.org/10.1016/j.febslet.2010.04.014.
(43) Woldring, D. R.; Holec, P. V.; Stern, L. A.; Du, Y.; Hackel, B. J. A Gradient of Sitewise Diversity Promotes Evolutionary Fitness for Binder Discovery in a Three-Helix Bundle Protein Scaffold. Biochemistry 2017, 56 (11), 1656-1671. https://doi.org/10.1021/acs.biochem.6b01142.
(44) Frejd, F. Y.; Kim, K.-T. Affibody Molecules as Engineered Protein Drugs. Exp. Mol. Med. 2017, 49 (3), e306-e306. https://doi.org/10.1038/emm.2017.35.
(45) Alexis, F.; Basto, P.; Levy-Nissenbaum, E.; Radovic-Moreno, A. F.; Zhang, L.; Pridgen, E.; Wang, A. Z.; Marein, S. L.; Westerhof, K.; Molnar, L. K.; Farokhzad, O. C. HER-2-Targeted Nanoparticle-Affibody Bioconjugates for Cancer Therapy. Chem Med Chem 2008, 3 (12), 1839-1843. https://doi.org/10.1002/cmdc.200800122.
(46) Persson, J.; Puuvuori, E.; Zhang, B.; Velikyan, I.; Åberg, O.; Müller, M.; Nygren, P.-Å.; Ståhl, S.; Korsgren, O.; Eriksson, O.; Löfblom, J. Discovery, Optimization and Biodistribution of an Affibody Molecule for Imaging of CD69. Sci. Rep. 2021, 11 (1), 19151. https://doi.org/10.1038/s41598-021-97694-6.
(47) Güler, R.; Svedmark, S. F.; Abouzayed, A.; Orlova, A.; Löfblom, J. Increasing Thermal Stability and Improving Biodistribution of VEGFR2-Binding Affibody Molecules by a Combination of in Silico and Directed Evolution Approaches. Sci. Rep. 2020, 10 (1), 18148. https://doi.org/10.1038/s41598-020-74560-5.
(48) Case, B. A.; Kruziki, M. A.; Stern, L. A.; Hackel, B. J. Evaluation of Affibody Charge Modification Identified by Synthetic Consensus Design in Molecular PET Imaging of Epidermal Growth Factor Receptor. Mol. Syst. Des. Eng. 2018, 3 (1), 171-182. https://doi.org/10.1039/C7ME00095B.
(49) Hackel, B. J.; Kapila, A.; Dane Wittrup, K. Picomolar Affinity Fibronectin Domains Engineered Utilizing Loop Length Diversity, Recursive Mutagenesis, and Loop Shuffling. J. Mol. Biol. 2008, 381 (5), 1238-1252. https://doi.org/10.1016/j.jmb.2008.06.051.
(50) Chao, G.; Lau, W. L.; Hackel, B. J.; Sazinsky, S. L.; Lippow, S. M.; Wittrup, K. D. Isolating and Engineering Human Antibodies Using Yeast Surface Display. Nat. Protoc. 2006, 1 (2), 755-769. https://doi.org/10.1038/nprot.2006.94.
(51) Mahmood, T.; Yang, P.-C. Western Blot: Technique, Theory, and Trouble Shooting. North Am. J. Med. Sci. 2012, 4 (9), 429-434. https://doi.org/10.4103/1947-2714.100998.
(52) Stern, L. A.; Csizmar, C. M.; Woldring, D. R.; Wagner, C. R.; Hackel, B. J. Titratable Avidity Reduction Enhances Affinity Discrimination in Mammalian Cellular Selections of Yeast-Displayed Ligands. ACS Comb. Sci. 2017, 19 (5), 315-323. https://doi.org/10.1021/acscombsci.6b00191.
(53) Colabroy, K. L.; Mayer, K. Benchtop Immobilized Metal Affinity Chromatography, Reconstitution and Assay of a Polyhistidine Tagged Metalloenzyme for the Undergraduate Laboratory. J. Vis. Exp. JoVE 2018, No. 138, 58012. https://doi.org/10.3791/58012.
(54) Robichon, C.; Luo, J.; Causey, T. B.; Benner, J. S.; Samuelson, J. C. Engineering Escherichia Coli BL21(DE3) Derivative Strains To Minimize E. Coli Protein Contamination after Purification by Immobilized Metal Affinity Chromatography ∇. Appl. Environ. Microbiol. 2011, 77 (13), 4634-4646. https://doi.org/10.1128/AEM.00119-11.
(55) Asiimwe, N.; Al Mazid, M. F.; Murale, D. P.; Kim, Y. K.; Lee, J.-S. Recent Advances in Protein Modifications Techniques for the Targeting N-Terminal Cysteine. Pept. Sci. 2022, 114 (3), e24235. https://doi.org/10.1002/pep2.24235.
(56) Phelps, E. A.; Enemchukwu, N. O.; Fiore, V. F.; Sy, J. C.; Murthy, N.; Sulchek, T. A.; Barker, T. H.; García, A. J. Maleimide Cross-Linked Bioactive PEG Hydrogel Exhibits Improved Reaction Kinetics and Cross-Linking for Cell Encapsulation and In Situ Delivery. Adv. Mater. 2012, 24 (1), 64-70. https://doi.org/10.1002/adma.201103574.
(57) Fisher, S. A.; Baker, A. E. G.; Shoichet, M. S. Designing Peptide and Protein Modified Hydrogels: Selecting the Optimal Conjugation Strategy. J. Am. Chem. Soc. 2017, 139 (22), 7416-7427. https://doi.org/10.1021/jacs.7b00513.
(58) Arbour, C. A.; Mendoza, L. G.; Stockdill, J. L. Recent Advances in the Synthesis of C-Terminally Modified Peptides. Org. Biomol. Chem. 2020, 18 (37), 7253-7272. https://doi.org/10.1039/d0ob01417f.
(59) Benavides, I.; Raftery, E. D.; Bell, A. G.; Evans, D.; Scott, W. A.; Houk, K. N.; Deming, T. J. Poly(Dehydroalanine): Synthesis, Properties, and Functional Diversification of a Fluorescent Polypeptide. J. Am. Chem. Soc. 2022, 144 (9), 4214-4223. https://doi.org/10.1021/jacs.2c00383.
(60) Nielsen, R. W.; Tachibana, C.; Hansen, N. E.; Winther, J. R. Trisulfides in Proteins. Antioxid. Redox Signal. 2011, 15 (1), 67-75. https://doi.org/10.1089/ars.2010.3677.
(61) Zeng, J.; Davies, M. J. Evidence for the Formation of Adducts and S-(Carboxymethyl)Cysteine on Reaction of α-Dicarbonyl Compounds with Thiol Groups on Amino Acids, Peptides, and Proteins. Chem. Res. Toxicol. 2005, 18 (8), 1232-1241. https://doi.org/10.1021/tx050074u.
(62) Greenfield, N. J. Using Circular Dichroism Spectra to Estimate Protein Secondary Structure. Nat. Protoc. 2006, 1 (6), 2876-2890. https://doi.org/10.1038/nprot.2006.202.
(63) Rodger, A.; Marshall, D. Beginners Guide to Circular Dichroism. The Biochemist 2021, 43 (2), 58-64. https://doi.org/10.1042/bio_2020_105.
(64) Mirdita, M.; Schütze, K.; Moriwaki, Y.; Heo, L.; Ovchinnikov, S.; Steinegger, M. ColabFold: Making Protein Folding Accessible to All. Nat. Methods 2022. https://doi.org/10.1038/s41592-022-01488-1.
(65) Mirdita, M.; Steinegger, M.; Söding, J. MMseqs2 Desktop and Local Web Server App for Fast, Interactive Sequence Searches. Bioinformatics 2019, 35 (16), 2856-2858. https://doi.org/10.1093/bioinformatics/bty1057.
(66) Mirdita, M.; von den Driesch, L.; Galiez, C.; Martin, M. J.; Söding, J.; Steinegger, M. Uniclust Databases of Clustered and Deeply Annotated Protein Sequences and Alignments. Nucleic Acids Res 2017, 45 (D1), D170-D176. https://doi.org/10.1093/nar/gkw1081.
(67) Evans, R.; O'Neill, M.; Pritzel, A.; Antropova, N.; Senior, A.; Green, T.; Zidek, A.; Bates, R.; Blackwell, S.; Yim, J.; Ronneberger, O.; Bodenstein, S.; Zielinski, M.; Bridgland, A.; Potapenko, A.; Cowie, A.; Tunyasuvunakool, K.; Jain, R.; Clancy, E.; Kohli, P.; Jumper, J.; Hassabis, D. Protein Complex Prediction with AlphaFold-Multimer. bioRxiv 2021. https://doi.org/10.1101/2021.10.04.463034v1.
(68) Mitchell, A. L.; Almeida, A.; Beracochea, M.; Boland, M.; Burgin, J.; Cochrane, G.; Crusoe, M. R.; Kale, V.; Potter, S. C.; Richardson, L. J.; Sakharova, E.; Scheremetjew, M.; Korobeynikov, A.; Shlemov, A.; Kunyavskaya, O.; Lapidus, A.; Finn, R. D. MGnify: The Microbiome Analysis Resource in 2020. Nucleic Acids Res 2019. https://doi.org/10.1093/nar/gkz1035.
(69) Jumper, J.; Evans, R.; Pritzel, A.; Green, T.; Figurnov, M.; Ronneberger, O.; Tunyasuvunakool, K.; Bates, R.; Žídek, A.; Potapenko, A.; Bridgland, A.; Meyer, C.; Kohl, S. A. A.; Ballard, A. J.; Cowie, A.; Romera-Paredes, B.; Nikolov, S.; Jain, R.; Adler, J.; Back, T.; Petersen, S.; Reiman, D.; Clancy, E.; Zielinski, M.; Steinegger, M.; Pacholska, M.; Berghammer, T.; Bodenstein, S.; Silver, D.; Vinyals, O.; Senior, A. W.; Kavukcuoglu, K.; Kohli, P.; Hassabis, D. Highly Accurate Protein Structure Prediction with AlphaFold. Nature 2021, 596 (7873), 583-589. https://doi.org/10.1038/s41586-021-03819-2.
(70) Nivón, L. G.; Moretti, R.; Baker, D. A Pareto-Optimal Refinement Method for Protein Design Scaffolds. PLOS ONE 2013, 8 (4), e59004. https://doi.org/10.1371/journal.pone.0059004.
(71) Tyka, M. D.; Keedy, D. A.; André, I.; DiMaio, F.; Song, Y.; Richardson, D. C.; Richardson, J. S.; Baker, D. Alternate States of Proteins Revealed by Detailed Energy Landscape Mapping. J. Mol. Biol. 2011, 405 (2), 607-618. https://doi.org/10.1016/j.jmb.2010.11.008.
(72) Conway, P.; Tyka, M. D.; DiMaio, F.; Konerding, D. E.; Baker, D. Relaxation of Backbone Bond Geometry Improves Protein Energy Landscape Modeling. Protein Sci. Publ. Protein Soc. 2014, 23 (1), 47-55. https://doi.org/10.1002/pro.2389.
(73) Pierce, B. G.; Wiehe, K.; Hwang, H.; Kim, B.-H.; Vreven, T.; Weng, Z. ZDOCK Server: Interactive Docking Prediction of Protein-Protein Complexes and Symmetric Multimers. Bioinformatics 2014, 30 (12), 1771-1773. https://doi.org/10.1093/bioinformatics/btu097.
(74) Schrödinger, LLC. The PyMOL Molecular Graphics System, Version 1.8, 2015.
(75) Rahman, M. S.; Akhtar, N.; Jamil, H. M.; Banik, R. S.; Asaduzzaman, S. M. TGF-β/BMP Signaling and Other Molecular Events: Regulation of Osteoblastogenesis and Bone Formation. Bone Res. 2015, 3, 15005. https://doi.org/10.1038/boneres.2015.5.
(76) Maguire, J. B.; Haddox, H. K.; Strickland, D.; Halabiya, S. F.; Coventry, B.; Griffin, J. R.; Pulavarti, S. V. S. R. K.; Cummins, M.; Thieker, D. F.; Klavins, E.; Szyperski, T.; DiMaio, F.; Baker, D.; Kuhlman, B. Perturbing the Energy Landscape for Improved Packing during Computational Protein Design. Proteins 2021, 89 (4), 436-449. https://doi.org/10.1002/prot.26030.
(77) Hagemans, D.; van Belzen, I. A. E. M.; Morán Luengo, T.; Rüdiger, S. G. D. A Script to Highlight Hydrophobicity and Charge on Protein Surfaces. Front. Mol. Biosci. 2015, 2.
(78) Martino, M. M.; Briquez, P. S.; Güç, E.; Tortelli, F.; Kilarski, W. W.; Metzger, S.; Rice, J. J.; Kuhn, G. A.; Müller, R.; Swartz, M. A.; Hubbell, J. A. Growth Factors Engineered for Super-Affinity to the Extracellular Matrix Enhance Tissue Healing. Science 2014, 343 (6173), 885-888. https://doi.org/10.1126/science.1247663.
(79) Boraschi-Diaz, I.; Wang, J.; Mort, J. S.; Komarova, S. V. Collagen Type I as a Ligand for Receptor-Mediated Signaling. Front. Phys. 2017, 5.
(80) Kotzsch, A.; Nickel, J.; Seher, A.; Heinecke, K.; van Geersdaele, L.; Herrmann, T.; Sebald, W.; Mueller, T. D. Structure Analysis of Bone Morphogenetic Protein-2 Type I Receptor Complexes Reveals a Mechanism of Receptor Inactivation in Juvenile Polyposis Syndrome*. J. Biol. Chem. 2008, 283 (9), 5876-5887. https://doi.org/10.1074/jbc.M706029200.
(81) Kirsch, T.; Nickel, J.; Sebald, W. BMP-2 Antagonists Emerge from Alterations in the Low-Affinity Binding Epitope for Receptor BMPR-II. EMBO J. 2000, 19 (13), 3314-3324. https://doi.org/10.1093/emboj/19.13.3314.
(82) Katagiri, T.; Tsukamoto, S. The Unique Activity of Bone Morphogenetic Proteins in Bone: A Critical Role of the Smad Signaling Pathway. Biol. Chem. 2013, 394 (6), 703-714. https://doi.org/10.1515/hsz-2012-0310.
(83) Karim, Md. S.; Madamanchi, A.; Dutko, J. A.; Mullins, M. C.; Umulis, D. M. Heterodimer-Heterotetramer Formation Mediates Enhanced Sensor Activity in a Biophysical Model for BMP Signaling. PLoS Comput. Biol. 2021, 17 (9), e1009422. https://doi.org/10.1371/journal.pcbi.1009422.
(84) Liu, R.; Ginn, S. L.; Lek, M.; North, K. N.; Alexander, I. E.; Little, D. G.; Schindeler, A. Myoblast Sensitivity and Fibroblast Insensitivity to Osteogenic Conversion by BMP-2 Correlates with the Expression of Bmpr-1a. BMC Musculoskelet. Disord. 2009, 10 (1), 51. https://doi.org/10.1186/1471-2474-10-51.
(85) Song, R.; Wang, D.; Zeng, R.; Wang, J. Synergistic Effects of Fibroblast Growth Factor-2 and Bone Morphogenetic Protein-2 on Bone Induction. Mol. Med. Rep. 2017, 16 (4), 4483-4492. https://doi.org/10.3892/mmr.2017.7183.
(86) Zernik, J.; Twarog, K.; Upholt, W. B. Regulation of Alkaline Phosphatase and Alpha 2(I) Procollagen Synthesis during Early Intramembranous Bone Formation in the Rat Mandible. Differ. Res. Biol. Divers. 1990, 44 (3), 207-215. https://doi.org/10.1111/j.1432-0436.1990.tb00619.x.
(87) Sepulveda, J. L. Chapter 10—Challenges in Routine Clinical Chemistry Analysis: Proteins and Enzymes. In Accurate Results in the Clinical Laboratory (Second Edition); Dasgupta, A., Sepulveda, J. L., Eds.; Elsevier, 2019; pp 141-163. https://doi.org/10.1016/B978-0-12-813776-5.00010-8.
(88) Zhang, J.; Lu, X.; Lei, Y.; Hou, X.; Wu, P. Exploring the Tunable Excitation of QDs to Maximize the Overlap with the Absorber for Inner Filter Effect-Based Phosphorescence Sensing of Alkaline Phosphatase. Nanoscale 2017, 9 (40), 15606-15611. https://doi.org/10.1039/C7NR03673F.
(89) Friess, W.; Uludag, H.; Foskett, S.; Biron, R.; Sargeant, C. Characterization of Absorbable Collagen Sponges as RhBMP-2 Carriers. Int. J. Pharm. 1999, 187 (1), 91-99. https://doi.org/10.1016/S0378-5173(99)00174-X.
(90) Yang, H. S.; La, W.-G.; Cho, Y.-M.; Shin, W.; Yeo, G.-D.; Kim, B.-S. Comparison between Heparin-Conjugated Fibrin and Collagen Sponge as Bone Morphogenetic Protein-2 Carriers for Bone Regeneration. Exp. Mol. Med. 2012, 44 (5), 350-355. https://doi.org/10.3858/emm.2012.44.5.039.
(91) Jansen, L. E.; Negrón-Piñeiro, L. J.; Galarza, S.; Peyton, S. R. Control of Thiol-Maleimide Reaction Kinetics in PEG Hydrogel Networks. Acta Biomater. 2018, 70, 120-128. https://doi.org/10.1016/j.actbio.2018.01.043.
(92) Guo, Y.; Gu, J.; Jiang, Y.; Zhou, Y.; Zhu, Z.; Ma, T.; Cheng, Y.; Ji, Z.; Jiao, Y.; Xue, B.; Cao, Y. Regulating the Homogeneity of Thiol-Maleimide Michael-Type Addition-Based Hydrogels Using Amino Biomolecules. Gels 2021, 7 (4), 206. https://doi.org/10.3390/gels7040206.
(93) Abouelmagd, S. A.; Sun, B.; Chang, A. C.; Ku, Y. J.; Yeo, Y. Release Kinetics Study of Poorly Water-Soluble Drugs from Nanoparticles: Are We Doing It Right?. ACS Publications. https://doi.org/10.1021/mp500817h.
(94) Jensen, S. S.; Jensen, H.; Møller, E. H.; Cornett, C.; Siepmann, F.; Siepmann, J.; Østergaard, J. In Vitro Release Studies of Insulin from Lipid Implants in Solution and in a Hydrogel Matrix Mimicking the Subcutis. Eur. J. Pharm. Sci. 2016, 81, 103-112. https://doi.org/10.1016/j.ejps.2015.10.011.
(95) Gstraunthaler, G.; Lindl, T.; van der Valk, J. A Plea to Reduce or Replace Fetal Bovine Serum in Cell Culture Media. Cytotechnology 2013, 65 (5), 791-793. https://doi.org/10.1007/s10616-013-9633-8.
(96) Metters, A.; Hubbell, J. Network Formation and Degradation Behavior of Hydrogels Formed by Michael-Type Addition Reactions. Biomacromolecules 2005, 6 (1), 290-301. https://doi.org/10.1021/bm049607o.
(97) Mellott, M. B.; Searcy, K.; Pishko, M. V. Release of Protein from Highly Cross-Linked Hydrogels of Poly(Ethylene Glycol) Diacrylate Fabricated by UV Polymerization. Biomaterials 2001, 22 (9), 929-941. https://doi.org/10.1016/s0142-9612(00)00258-1.
(98) Leach, J. B.; Schmidt, C. E. Characterization of Protein Release from Photocrosslinkable Hyaluronic Acid-Polyethylene Glycol Hydrogel Tissue Engineering Scaffolds. Biomaterials 2005, 26 (2), 125-135. https://doi.org/10.1016/j.biomaterials.2004.02.018.
(99) Zustiak, S. P.; Leach, J. B. Characterization of Protein Release from Hydrolytically Degradable Poly(Ethylene Glycol) Hydrogels. Biotechnol. Bioeng. 2011, 108 (1), 197-206. https://doi.org/10.1002/bit.22911.
(100) Wang, Y.; Delgado-Fukushima, E.; Fu, R. X.; Doerk, G. S.; Kim, J. Controlling Drug Absorption, Release and Erosion of Photopatterned Protein Engineered Hydrogels.
(101) Vermonden, T.; Censi, R.; Hennink, W. E. Hydrogels for Protein Delivery. Chem. Rev. 2012, 112 (5), 2853-2888. https://doi.org/10.1021/cr200157d.
(102) Gregoritza, M.; Messmann, V.; M. Goepferich, A.; P. Brandl, F. Design of Hydrogels for Delayed Antibody Release Utilizing Hydrophobic Association and Diels-Alder Chemistry in Tandem. J. Mater. Chem. B 2016, 4 (19), 3398-3408. https://doi.org/10.1039/C6TB00223D.
(103) Quaas, B.; Burmeister, L.; Li, Z.; Satalov, A.; Behrens, P.; Hoffmann, A.; Rinas, U. Stability and Biological Activity of E. Coli Derived Soluble and Precipitated Bone Morphogenetic Protein-2. Pharm. Res. 2019, 36 (12), 184. https://doi.org/10.1007/s11095-019-2705-5.
(104) Sundermann, J.; Zagst, H.; Kuntsche, J.; Wätzig, H.; Bunjes, H. Bone Morphogenetic Protein 2 (BMP-2) Aggregates Can Be Solubilized by Albumin—Investigation of BMP-2 Aggregation by Light Scattering and Electrophoresis. Pharmaceutics 2020, 12 (12), 1143. https://doi.org/10.3390/pharmaceutics12121143.
(105) Ritger, P. L.; Peppas, N. A. A Simple Equation for Description of Solute Release II. Fickian and Anomalous Release from Swellable Devices. J. Controlled Release 1987, 5 (1), 37-42. https://doi.org/10.1016/0168-3659(87)90035-6.
(106) Lowe, S.; O'Brien-Simpson, N. M.; Connal, L. A. Antibiofouling Polymer Interfaces: Poly(Ethylene Glycol) and Other Promising Candidates. Polym. Chem. 2014, 6 (2), 198-212. https://doi.org/10.1039/C4PY01356E.
(107) Kuo, J.-H. S. Effect of Pluronic-Block Copolymers on the Reduction of Serum-Mediated Inhibition of Gene Transfer of Polyethyleneimine-DNA Complexes. Biotechnol. Appl. Biochem. 2003, 37 (3), 267-271. https://doi.org/10.1042/BA20020123.
(108) Fung, S. L.; Wu, X.; Maceren, J. P.; Mao, Y.; Kohn, J. In Vitro Evaluation of Recombinant Bone Morphogenetic Protein-2 Bioactivity for Regenerative Medicine. Tissue Eng. Part C Methods 2019, 25 (9), 553-559. https://doi.org/10.1089/ten.tec.2019.0156.
(109) Hettiaratchi, M. H.; Krishnan, L.; Rouse, T.; Chou, C.; McDevitt, T. C.; Guldberg, R. E. Heparin-Mediated Delivery of Bone Morphogenetic Protein-2 Improves Spatial Localization of Bone Regeneration. Sci. Adv. 2020, 6 (1), eaay1240. https://doi.org/10.1126/sciadv.aay1240.
(110) Jung, T.; Lee, J. H.; Park, S.; Kim, Y.-J.; Seo, J.; Shim, H.-E.; Kim, K.-S.; Jang, H.-S.; Chung, H.-M.; Oh, S.-G.; Moon, S.-H.; Kang, S.-W. Effect of BMP-2 Delivery Mode on Osteogenic Differentiation of Stem Cells. Stem Cells Int. 2017, 2017, 7859184. https://doi.org/10.1155/2017/7859184.
(111) Zhao, B.; Katagiri, T.; Toyoda, H.; Takada, T.; Yanai, T.; Fukuda, T.; Chung, U.; Koike, T.; Takaoka, K.; Kamijo, R. Heparin Potentiates the in Vivo Ectopic Bone Formation Induced by Bone Morphogenetic Protein-2*. J. Biol. Chem. 2006, 281 (32), 23246-23253. https://doi.org/10.1074/jbc.M511039200.
(112) Van Hove, A. H.; Beltejar, M.-J.; Benoit, D. S. W. Development and in Vitro Assessment of Enzymatically-Responsive Poly(Ethylene Glycol) Hydrogels for the Delivery of Therapeutic Peptides. Biomaterials 2014, 35 (36), 9719-9730. https://doi.org/10.1016/j.biomaterials.2014.08.019.
(113) Shih, H.; Lin, C.-C. Crosslinking and Degradation of Step-Growth Hydrogels Formed by Thiol-Ene Photo-Click Chemistry. Biomacromolecules 2012, 13 (7), 2003-2012. https://doi.org/10.1021/bm300752j.
(114) Gawade, P. M.; Shadish, J. A.; Badeau, B. A.; DeForest, C. A. Logic-Based Delivery of Site-Specifically Modified Proteins from Environmentally Responsive Hydrogel Biomaterials. Adv. Mater. 2019, 31 (33), 1902462. https://doi.org/10.1002/adma.201902462.
(115) Marty, M. T.; Baldwin, A. J.; Marklund, E. G.; Hochberg, G. K. A.; Benesch, J. L. P.; Robinson, C. V. Bayesian Deconvolution of Mass and Ion Mobility Spectra: From Binary Interactions to Polydisperse Ensembles. Anal. Chem. 2015, 87 (8), 4370-4376. https://doi.org/10.1021/acs.analchem.5b00140.
(116) Khatib, F.; Cooper, S.; Tyka, M. D.; Xu, K.; Makedon, I.; Popović, Z.; Baker, D.; Players, F. Algorithm Discovery by Protein Folding Game Players. Proc. Natl. Acad. Sci. 2011, 108 (47), 18949-18953. https://doi.org/10.1073/pnas.1115898108.
(117) Keller, S.; Nickel, J.; Zhang, J.-L.; Sebald, W.; Mueller, T. D. Molecular Recognition of BMP-2 and BMP Receptor IA. Nat. Struct. Mol. Biol. 2004, 11 (5), 481-488. https://doi.org/10.1038/nsmb756.
(118) Guo, J.; Liu, B.; Thorikay, M.; Yu, M.; Li, X.; Tong, Z.; Salmon, R. M.; Read, R. J.; ten Dijke, P.; Morrell, N. W.; Li, W. Crystal Structures of BMPRII Extracellular Domain in Binary and Ternary Receptor Complexes with BMP10. Nat. Commun. 2022, 13 (1), 2395. https://doi.org/10.1038/s41467-022-30111-2.

In view of the many possible embodiments to which the principles of the disclosure may be applied, it should be recognized that the illustrated embodiments are only examples of the disclosure and should not be taken as limiting the scope of the invention. Rather, the scope of the invention is defined by the following claims. We therefore claim as our invention all that comes within the scope and spirit of these claims.

We claim:

1. A composition comprising:
a hydrogel;
one or more proteins; and
one or more affibodies; wherein the one or more affibodies are specific for the one or more proteins.

2. The composition of claim 1, wherein the one or more target proteins are non-covalently bound to the one or more affibodies.

3. The composition of claim 1, further comprising a pharmaceutically acceptable carrier.

4. The composition of claim 1, wherein the one or more proteins comprise one or more of bone morphogenetic protein 2 (BMP-2), vascular endothelial growth factor (VEGF), fibroblast growth factor 2 (FGF-2), platelet-derived growth factor (PDGF), granulocyte-macrophage colony-stimulating factor (GM-CSF), inteleukin-4 (IL-4), and glial derived neurotrophic factor (GDNF).

5. The composition of claim 4, wherein the one or more proteins further comprise one or more of collagen I, collagen III, and monocyte chemoattractant protein-1 (MCP-1).

6. The composition of claim 1, wherein the hydrogel comprises at least two different affibodies, wherein the at least two affibodies are specific for at least two of BMP-2, VEGF, FGF2, PDGF, GM-CSF, IL-4, and GDNF.

7. The composition of claim 1, wherein the hydrogel comprises affibodies specific for:
VEGF, FGF2, and PDGF-BB;
GM-CSF;
GDNF;
BMP-2;
BMP-2 and IL-4;
VEGF, FGF2, PDGF-BB, and BMP-2;
PDGF-BB and VEGF;
GM-CSF and IL-4;
GM-CSF, IL-4 and MCP-1; or
GM-CSF, IL-4, and BMP-2.

8. The composition of claim 1, wherein the one or more affibodies comprise one or more of SEQ ID NOS: 1-74, wherein SEQ ID NOS: 1-74 optionally further include a C-terminal Cys, Lys, Tyr, Try, or Phe.

9. The composition of claim 1, wherein the one or more affibodies comprise one or more of SEQ ID NOS: 1, 2, 3, 12, 13, 14, 20, 21, 22, 42, 43, 44, 57, 58, 59, 60, 61, 62, 63, and 64 and optionally an additional C-terminal Cys, Lys, Tyr, Try, or Phe.

10. The composition of claim 1, wherein the hydrogel comprises hyaluronic acid (HA), polyethylene glycol (PEG), PEG-Maleimide (PEG-Mal), modified hyaluronic acid, thiolated poly(E-caprolactone) (PCL-SH), thiolated poly(lactide-co-glycolide) (PLGA-SH), thiolated silk-firbroin, modified gelatin (methacrylate (GelMA), oxidized gelatin, gelatin norbornene), collagen, or combinations thereof.

11. The composition of claim 1, wherein the one or more affibodies include at least three different affibodies specific for one or more of BMP-2, VEGF, FGF2, PDGF, GM-CSF, IL-4, and GDNF, wherein the at least three different affibodies each have different dissociation constants (K_{D}) for the protein.

12. A method of treating a subject
administering an effective amount of the composition of claim 1 to the subject, thereby treating the subject.

13. The method of claim 12, wherein the subject has a bone injury, and the method includes administration of the composition to the site of injury or systemic administration, and the composition includes one or more BMP-2 affibodies, one or more IL-4 affibodies, and/or one or more GM-CSF affibodies.

14. The method of claim 12, wherein the subject has a vascular disease, and the method includes administration of the composition to the site of injury or systemic administration, and the composition includes one or more VEGF affibodies, one or more FGF-2 affibodies, one or more PDGF affibodies, and/or one or more GM-CSF affibodies.

15. The method of claim 14, wherein the vascular disease is a wound, peripheral artery disease, diabetic ulcer, or critical limb ischemia.

16. The method of claim 12, wherein the subject has a neurological disease or injury, and the method includes administration of the composition to the site of disease injury or systemic administration, and the composition includes one or more GDNF affibodies.

17. The method of claim 12, wherein the administering is surgical administration or injection.

18. An isolated affibody
comprising at least 90% sequence identity to any one of SEQ ID NOS: 1-74;
comprising at least 90% sequence identity to any one of SEQ ID NOS: 1-74 and further comprising a C-terminal Cys, Lys, Tyr, Try, or Phe;
comprising of any one of SEQ ID NOS: 1-74;
comprising of any one of SEQ ID NOS: 1-74 and further comprising a C-terminal Cys, Lys, Tyr, Try, or Phe;
consisting of any one of SEQ ID NOS: 1-74; or
consisting of any one of SEQ ID NOS: 1-74 and a C-terminal cysteine.

19. The isolated affibody of claim 18, wherein the affibody is 58, 59, 60, or 65 amino acids in length.

20. The isolated affibody of claim 18, wherein the affibody comprises 1, 2, 3, 4, 5 or 6 conservative amino acid substitutions.


SEQUENCE LISTING (76 sequences)

SEQ ID NO: 1 - protein, 58 aa, synthetic construct
AEAKYYKEVSSAATQIRYLPNLTAFQKAAFYAALLDDPSQSSELLSEAKKLNDSQAPK

SEQ ID NO: 2 - protein, 58 aa, synthetic construct
AEAKYAKEQFNAYVVIFYLPNLTASQKAAFVDALSNDPSQSSELLSEAKKLNDSQAPK

SEQ ID NO: 3 - protein, 58 aa, synthetic construct
AEAKYYKEGDNAYNVIYGLPNLTRPQRLAFIVALFNDPSQSSELLSEAKKLNDSQAPK

SEQ ID NO: 4 - protein, 58 aa, synthetic construct
AEAKYNKEVTAAANSIWVLPNLTGDQKAAFFEALLDDPSQSSELLSEAKKLNDSQAPK

SEQ ID NO: 5 - protein, 58 aa, synthetic construct
AEAKYTKEGFDAYDVIDNLPNLTLDQRNAFVYALFNDPSQSSELLSEAKKLNDSQAPK

SEQ ID NO: 6 - protein, 58 aa, synthetic construct
AEAKYYKEWLDADMSIRSLPNLTGYQIRAFIAALGNDPSQSSELLSEAKKLNDSQAPK

SEQ ID NO: 7 - protein, 58 aa, synthetic construct
AEAKYYKERRAAAVVIFYLPNLTRVQKGAFIEALDDDPSQSSELLSEAKKLNDSQAPK

SEQ ID NO: 8 - protein, 58 aa, synthetic construct
AEAKYAKERLNAIYVINDLPNLTQGQRVAFARALYNDPSQSSELLSEAKKLNDSQAPK

SEQ ID NO: 9 - protein, 58 aa, synthetic construct; identical to SEQ ID NO: 2

SEQ ID NO: 10 - protein, 58 aa, synthetic construct
AEAKYYKEWVNAYDQIRVLPNLTRFQRLAFYRALYNDPSQSSELLSEAKKLNDSQAPK

SEQ ID NO: 11 - protein, 58 aa, synthetic construct; identical to SEQ ID NO: 6

SEQ ID NO: 12 - protein, 58 aa, synthetic construct
AEAKYTKELFNAVGEITALPNLTRYHLYAFYYALLNDPSQSSELLSEAKKLNDSQAPK

SEQ ID NO: 13 - protein, 58 aa, synthetic construct
AEAKYNKEWFAADLSIGFLPNLTLDQLYAFVFALYDDPSQSSELLSEAKKLNDSQAPK

SEQ ID NO: 14 - protein, 58 aa, synthetic construct
AEAKYAKEGLNAYLSIRWLPNLTGDQMYAFISALLDDPSQSSELLSEAKKLNDSQAPK

SEQ ID NO: 15 - protein, 58 aa, synthetic construct
AEAKYTKEGFNAYDEIDNLPNLTLDQRNAFVYALFNDPSQSSELLSEAKKLNDSQAPK

SEQ ID NO: 16 - protein, 58 aa, synthetic construct; identical to SEQ ID NO: 12

SEQ ID NO: 17 - protein, 58 aa, synthetic construct
AEAKYNKEVGTANFEIVLLPNLTLYQMLAFIKALVNDPSQSSELLSEAKKLNDSQAPK

SEQ ID NO: 18 - protein, 58 aa, synthetic construct
AEAKYNKEWYNAISVIFYLPNLTGFQRAAFVDALGDDPSQSSELLSEAKKLNDSQAPK

SEQ ID NO: 19 - protein, 58 aa, synthetic construct
AEAKYYKEGFYANFVIGALPNLTLVQRAAFYFALLNDPSQSSELLSEAKKLNDSQAPK

SEQ ID NO: 20 - protein, 58 aa, synthetic construct
AEAKYYKEGATAYRVIEYLPNLTGAQKAAFIDALYNDPSQSSELLSEAKKLNDSQAPK

SEQ ID NO: 21 - protein, 58 aa, synthetic construct; identical to SEQ ID NO: 5

SEQ ID NO: 22 - protein, 58 aa, synthetic construct
AEAKYNKEWYDAVFVIGSLPNLTEDQKDAFSDALVDDPSQSSELLSEAKKLNDSQAPK

SEQ ID NO: 23 - protein, 58 aa, synthetic construct
AEAKYYKEWNAAYVVINGLPNLTRRQREAFVHALVDDPSQSSELLSEAKKLNDSQAPK

SEQ ID NO: 24 - protein, 58 aa, synthetic construct
AEAKYYKERYAANYSIWVLPNLTLLQRFAFFFALSNDPSQSSELLSEAKKLNDSQAPK

SEQ ID NO: 25 - protein, 58 aa, synthetic construct
AEAKYAKELDDAFFEIASLPNLTGFQLHAFAVALGNDPSQSSELLSEAKKLNDSQAPK

SEQ ID NO: 26 - protein, 58 aa, synthetic construct
AEAKYNKERDSAYSVIWGLPNLTDSQKAAFGYALYNDPSQSSELLSEAKKLNDSQAPK

SEQ ID NO: 27 - protein, 58 aa, synthetic construct
AEAKYAKELEAANMVIVDLPNLTHGQKVAFLVALFNDPSQSSELLSEAKKLNDSQAPK

SEQ ID NO: 28 - protein, 58 aa, synthetic construct
AEAKYNKEWYDAILEIGFLPNLTGHQRDAFSDALVDDPSQSSELLSEAKKLNDSQAPK

SEQ ID NO: 29 - protein, 58 aa, synthetic construct
AEAKYNKEQDSAYSVIWGLPNLTESQKAAFGYALYDDPSQSSELLSEAKKLNDSQAPK

SEQ ID NO: 30 - protein, 58 aa, synthetic construct; identical to SEQ ID NO: 4

SEQ ID NO: 31 - protein, 58 aa, synthetic construct
AEAKYAKEWFYAYHVIYDLPNLTGFQKHAFYLALYDDPSQSSELLSEAKKLNDSQAPK

SEQ ID NO: 32 - protein, 58 aa, synthetic construct; identical to SEQ ID NO: 4

SEQ ID NO: 33 - protein, 58 aa, synthetic construct
AEAKYAKEGATAFGSIPYLPNLTDVQRYAFIVALLDDPSQSSELLSEAKKLNDSQAPK

SEQ ID NO: 34 - protein, 58 aa, synthetic construct
AEAKYTKEWYAAVVQIGYLPNLTAFQRAAFSFALSNDPSQSSELLSEAKKLNDSQAPK

SEQ ID NO: 35 - protein, 58 aa, synthetic construct
AEAKYTKERDDASLEIAYLPNLTPYQLMAFFFALSNDPSQSSELLSEAKKLNDSQAPK

SEQ ID NO: 36 - protein, 58 aa, synthetic construct
AEAKYAKEWTNAFVSIVCLPNLTAVQREAFVLALVDDPSQSSELLSEAKKLNDSQAPK

SEQ ID NO: 37 - protein, 58 aa, synthetic construct
AEAKYAKEWEDAINEIWCLPNLTEYQRIAFVSALYNDPSQSSELLSEAKKLNDSQAPK

SEQ ID NO: 38 - protein, 58 aa, synthetic construct
AEAKYAKELLNAFDEIYGLPNLTVGQRMAFCDALINDPSQSSELLSEAKKLNDSQAPK

SEQ ID NO: 39 - protein, 58 aa, synthetic construct
AEAKYYKEWYDAFVVIDALPNLTAYQREAFIFALVNDPSQSSELLSEAKKLNDSQAPK

SEQ ID NO: 40 - protein, 58 aa, synthetic construct
AEAKYYKEWVDAYLVIDSLPNLTRLQVEAFVFALVNDPSQSSELLSEAKKLNDSQAPK

SEQ ID NO: 41 - protein, 58 aa, synthetic construct
AEAKYTKEVDYAACVIAYLPNLTGVQVYAFYRALADDPSQSSELLSEAKKLNDSQAPK

SEQ ID NO: 42 - protein, 58 aa, synthetic construct
AEAKYTKEGSDAFDVIVLLPNLTRDQRDAFLYALLDDPSQSSELLSEAKKLNDSQAPK

SEQ ID NO: 43 - protein, 58 aa, synthetic construct
AEAKYAKEWLSADYVIICLPNLTLDQMVAFYDALFNDPSQSSELLSEAKKLNDSQAPK

SEQ ID NO: 44 - protein, 58 aa, synthetic construct
AEAKYNKEVFDADCSIWYLPNLTRYQISAFQSALDDDPSQSSELLSEAKKLNDSQAPK

SEQ ID NO: 45 - protein, 58 aa, synthetic construct
AEAKYTKEGCDAYTEIVDLPNLTGYQRRAFYWALENDPSQSSELLSEAKKLNDSQAPK

SEQ ID NO: 46 - protein, 58 aa, synthetic construct
AEAKYNKEMPDANCQIAFLPNLTQYQVPAFIYALCNDPSQSSELLSEAKKLNDSQAPK

SEQ ID NO: 47 - protein, 58 aa, synthetic construct
AEAKYNKEGEDATTQIGSLPNLTQAQKHAFAVALGNDPSQSSELLSEAKKLNDSQAPK

SEQ ID NO: 48 - protein, 58 aa, synthetic construct
AEAKYSKEGFYADWVIPVLPNLTRKQRVAFHDALHNDPSQSSELLSEAKKLNDSQAPK

SEQ ID NO: 49 - protein, 58 aa, synthetic construct
AEAKYAKEWLDAIDVIGYLPNLTDFQRGAFYDALNDDPSQSSELLSEAKKLNDSQAPK

SEQ ID NO: 50 - protein, 58 aa, synthetic construct
AEAKYYKEGYNAIVEIRCLPNLTDCQVAAFIDALDDDPSQSSELLSEAKKLNDSQAPK

SEQ ID NO: 51 - protein, 58 aa, synthetic construct
AEAKYAKELDAAYVVIYFLPNLTHCQMVAFLHALSDDPSQSSELLSEAKKLNDSQAPK

SEQ ID NO: 52 - protein, 58 aa, synthetic construct
AEAKYSKEVYSAYDVIFALPNLTQYQVLAFFDALCDDPSQSSELLSEAKKLNDSQAPK

SEQ ID NO: 53 - protein, 58 aa, synthetic construct
AEAKYAKERLTAVCSIVALPNLTEGQMVAFDDALHDDPSQSSELLSEAKKLNDSQAPK

SEQ ID NO: 54 - protein, 58 aa, synthetic construct
AEAKYAKEGFNAVNVIWPLPNLTADQVCAFICALADDPSQSSELLSEAKKLNDSQAPK

SEQ ID NO: 55 - protein, 58 aa, synthetic construct
AEAKYAKEGCTAFLEIAALPNLTGYQRDAFIEALFDDPSQSSELLSEAKKLNDSQAPK

SEQ ID NO: 56 - protein, 58 aa, synthetic construct; identical to SEQ ID NO: 42

SEQ ID NO: 57 - protein, 58 aa, synthetic construct
AEAKYYKEWDSASDSIGFLPNLTRAQMVAFFAALFNDPSQSSELLSEAKKLNDSQAPK

SEQ ID NO: 58 - protein, 58 aa, synthetic construct
AEAKYAHELWEADWEITNLPNLSPDQLMAFYMALWDDPSQSSELLSEAKKLNDSQAPK

SEQ ID NO: 59 - protein, 58 aa, synthetic construct
AEAKYAFELWEAQHEIQQLPNLRPDQIAAFAMALYDDPSQSSELLSEAKKLNDSQAPK

SEQ ID NO: 60 - protein, 58 aa, synthetic construct
AEAKYAKELDDASVEIWDLPNLTPCQKVAFFVALYDDPSQSSELLSEAKKLNDSQAPK

SEQ ID NO: 61 - protein, 60 aa, synthetic construct
AEAKYNKELDAADADVEIWLLPNLTLDQLLAFIAALFNDPSQSSELLSEAKKLNDSQAPK

SEQ ID NO: 62 - protein, 58 aa, synthetic construct
AEAKYTKELSDANAEIWSLPNLTVDQLVAFIFALWDDPSQSSELLSEAKKLNDSQAPK

SEQ ID NO: 63 - protein, 58 aa, synthetic construct
AEAKYSKEQSNAYASITDLPNLTRLQKLAFWVALFNDPSQSSELLSEAKKLNDSQAPK

SEQ ID NO: 64 - protein, 58 aa, synthetic construct
AERKYHWELLVAFMEIQSLPNLTKDQITQFMAALEDDPSQSSELLSEAKKLNDSQAPK

SEQ ID NO: 65 - protein, 58 aa, synthetic construct
AEAKYNKEQVYASDSIQVLPNLTATQRVAFDPALHNDPSQSSELLSEAKKLNDSQAPK

SEQ ID NO: 66 - protein, 58 aa, synthetic construct
AEAKYNKEKPNAVGEISVLPNLTEFQMVAFIFALVNDPSQSSELLSEAKKLNDSQAPK

SEQ ID NO: 67 - protein, 58 aa, synthetic construct
AEAKYAKEWTTANYSIGVLPNLTLTQRYAFETALFDDPSQSSELLSEAKKLNDSQAPK

SEQ ID NO: 68 - protein, 58 aa, synthetic construct
AEAKYTKERHDATLVIHVLPNLTDARILAFIVALSNDPSQSSELLSEAKKLNDSQAPK

SEQ ID NO: 69 - protein, 58 aa, synthetic construct
AEAKYNKERSNASFEILVLPNLTGIQKGAFFAALPDDPSQSSELLSEAKKLNDSQAPK

SEQ ID NO: 70 - protein, 58 aa, synthetic construct
AEAKYSKEWYDAYLVIFVLPNLTQFQRPAFPPALKNDPSQSSELLSEAKKLNDSQAPK

SEQ ID NO: 71 - protein, 65 aa, synthetic construct
AEAKYYKEVSSAATQIRYLPNLTAFQKAAFYAALLDDPSQSSELLSEAKKLNDSQAPKHHHHHHC

SEQ ID NO: 72 - protein, 65 aa, synthetic construct
AEAKYAKEQFNAYVVIFYLPNLTASQKAAFVDALSNDPSQSSELLSEAKKLNDSQAPKHHHHHHC

SEQ ID NO: 73 - protein, 65 aa, synthetic construct
AEAKYYKEGDNAYNVIYGLPNLTRPQRLAFIVALFNDPSQSSELLSEAKKLNDSQAPKHHHHHHC

SEQ ID NO: 74 - protein, 65 aa, synthetic construct
AEAKYTKELFNAVGEITALPNLTRYHLYAFYYALLNDPSQSSELLSEAKKLNDSQAPKHHHHHHC

SEQ ID NO: 75 - DNA, 20 bp, synthetic construct
  source 1..20: mol_type = other DNA
ccctcaacaactagcaaagg

SEQ ID NO: 76 - DNA, 24 bp, synthetic construct
  source 1..24: mol_type = other DNA
atgtgtaaagttggtaacggaacg